(19) United States
(12) Patent Application Publication
(10) Pub. No.: US 2023/0183740 A1
(43) Pub. Date: Jun. 15, 2023

(54) VIRAL VECTOR FOR COMBINATION THERAPY

(71) Applicant: Solid Biosciences Inc., Charlestown, MA (US)

(72) Inventors: Fatih Ozsolak, Quincy, MA (US); Eric Reyes, Boston, MA (US); Meghan S. Soustek-Kramer, Boston, MA (US); Sarath Chandra Mandava, Malden, MA (US); Joel Schneider, Stoneham, MA (US)

(21) Appl. No.: 17/791,840

(22) PCT Filed: Jan. 11, 2021
(86) PCT No.: PCT/US21/12967

§ 371 Date: Jul. 8, 2022

Related U.S. Application Data
(60) Provisional application No. 62/959,256, filed on Jan. 10, 2020.
(60) Provisional application No. 62/962,306, filed on Jan. 17, 2020.

(51) Int. Cl. C12N 15/86; C12N 15/11

(52) U.S. Cl. CPC C12N 15/86; C12N 15/11; C12N 2310/11; C12N 2750/14143; C12N 2310/141; C12N 2310/20; C12N 2310/531

(57) ABSTRACT

The invention described herein provides gene therapy vectors, such as adeno-associated virus (AAV) vectors, that co-express two or more GOIs. The vectors of the invention can be broadly used to treat a number of genetic disorders such as trinucleotide repeat expansion disorders.

### REFERENCE TO RELATED APPLICATION

This application claims priority to U.S. Provisional Patent Application Nos. 62/959,256, filed on Jan. 10, 2020, and 62/962,306, filed on Jan. 17, 2020, the entire contents of which are incorporated herein by reference.

This application also incorporates by reference U.S. Provisional Patent Application No. 62/778,646, filed on Dec. 12, 2018, and International Patent Application No. PCT/US2019/065718, filed on Dec. 11, 2019, which claims priority to U.S. Provisional Patent Application No. 62/778,646, filed on Dec. 12, 2018.

### REFERENCE TO SEQUENCE LISTING

The instant application contains a Sequence Listing file which has been submitted electronically in ASCII format and is hereby incorporated by reference in its entirety. Said ASCII copy, created on Dec. 28, 2022, is named 129159-11103_SL.txt and is 39,009 bytes in size. The Sequence Listing file is part of the specification, and is incorporated in its entirety by reference herein.

### BACKGROUND OF THE INVENTION

Muscular dystrophy (MD) is a group of diseases that cause progressive weakness and loss of muscle mass. In muscular dystrophy, abnormal genes (mutant genes) produce no functional wild-type proteins needed to form healthy muscle.

Muscular dystrophies have serious debilitating impacts on quality of life of affected patients. Duchenne type muscular dystrophy (DMD) is one of the most devastating muscle diseases affecting 1 in 5,000 newborn males. It is the most well-characterized muscular dystrophy, resulting from mutations in genes encoding members of the dystrophin-associated protein complex (DAPC). These MDs result from membrane fragility associated with the loss of sarcolemmal-cytoskeleton tethering by the DAPC.

Specifically, DMD is caused by mutations in the DMD gene, leading to reductions in DMD mRNA and the absence of dystrophin or functional dystrophin, a 427 kDa sarcolemmal protein associated with the dystrophin-associated protein complex (DAPC) (Hoffman et al., Cell 51(6):919-928, 1987). The DAPC is composed of multiple proteins at the muscle sarcolemma that form a structural link between the extra-cellular matrix (ECM) and the cytoskeleton via dystrophin, an actin binding protein, and alpha-dystroglycan, a laminin-binding protein. These structural links act to stabilize the muscle cell membrane during contraction, and protect against contraction-induced damage.

Loss of dystrophin as a result of DMD gene mutations disrupts the dystrophin glycoprotein complex, leading to increased muscle membrane fragility. A cascade of events including influx of calcium into the sarcoplasm, activation of proteases and proinflammatory cytokines, and mitochondrial dysfunction results in progressive muscle degeneration. In addition, displacement of neuronal nitric oxide synthase (nNOS) contributes to tissue ischemia, increased oxidative stress, and reparative failure. Disease progression is characterized by increasing muscle necrosis, fibrosis, and fatty tissue replacement and a greater degree of fiber size variation seen in subsequent muscle biopsies.

Accumulated evidence suggests that abnormal elevation of intracellular Ca²⁺ (Ca²⁺ᵢ) is an important, early pathogenic event that initiates and perpetuates disease progression in DMD. The normal function of sarco/endoplasmic reticulum Ca²⁺ ATPase (SERCA) pump accounts for >70% of Ca²⁺ removal from the cytosol and proper muscle contraction. Reduction in SERCA activity therefore has been considered as a primary cause of Ca²⁺ᵢ overload and muscle dysfunction in DMD.

Currently there is no cure for DMD. The standard of care includes administering corticosteroids (such as prednisone or deflazacort) to stabilize muscle strength and function, prolonging independent ambulation, and delaying scoliosis and cardiomyopathy; bisphosphonates; and denosumab and recombinant parathyroid hormones.

With the advent of gene therapy, research and clinical trials for DMD treatment has focused on gene replacement or other genetic therapies aimed to at least partially restore dystrophin function. These include supplying a functional copy of the dystrophin gene, such as a dystrophin minigene, or repairing a defective dystrophin gene product by exon skipping and nonsense mutation suppression.

However, due to the broad range of effects cause by the dystrophin mutation, there is a need to treat other secondary symptoms associated with the primary dystrophin mutation.

For example, loss of dystrophin leads to the loss of the dystrophin-associated protein complex (DAPC), which in turn leads to the production of nitric oxide (NO) by nNOS, and abnormal N-nitrosylation of HDAC2. Such abnormally N-nitrosylated HDAC2 dissociates from the chromatin, and releases the inhibition of a cascade of specific microRNAs which in turn lead to a slew of downstream events such as fibrosis and increased oxidative stress.

In particular, with respect to fibrosis, with dystrophin loss, membrane fragility results in sarcolemmal tears and an influx of calcium, triggering calcium-activated proteases and segmental fiber necrosis (Straub et al., Curr. Opin. Neurol. 10(2): 168-175, 1997). This uncontrolled cycle of muscle degeneration and regeneration ultimately exhausts the muscle stem cell population (Sacco et al., Cell 143(7): 1059-1071, 2010; Wallace et al., Annu Rev Physiol 71:37-57, 2009), resulting in progressive muscle weakness, endomysial inflammation, and fibrotic scarring.

Without membrane stabilization from dystrophin or a micro-dystrophin, DMD will manifest uncontrolled cycles of tissue injury and repair, and ultimately replace lost muscle fibers with fibrotic scar tissue through connective tissue proliferation.

Muscle biopsies taken at the earliest age of diagnosis of DMD (e.g., between 4-5 years old) reveal prominent connective tissue proliferation. Muscle fibrosis is deleterious in multiple ways. It reduces normal transit of endomysial nutrients through connective tissue barriers, reduces the blood flow and deprives muscle of vascular-derived nutritional constituents, and functionally contributes to early loss of ambulation through limb contractures. Over time, treatment challenges multiply as a result of marked fibrosis in muscle. This can be observed in muscle biopsies comparing connective tissue proliferation at successive time points. The process continues to exacerbate leading to loss of ambulation and accelerating out of control, especially in wheelchair-dependent patients.

Thus fibrotic infiltration is profound in DMD, and is a significant impediment to any potential therapy. In this regard, gene replacement therapy alone is usually hampered by the severity of fibrosis, already present in very young children with DMD.

Fibrosis is characterized by the excessive deposits of ECM matrix proteins, including collagen and elastin. ECM proteins are primarily produced from cytokines such as TGF that is released by activated fibroblasts responding to stress and inflammation. Although the primary pathological feature of DMD is myofiber degeneration and necrosis, fibrosis as a pathological consequence has equal repercussions. The over-production of fibrotic tissue restricts muscle regeneration and contributes to progressive muscle weakness in the DMD patient.

In one study, the presence of fibrosis on initial DMD muscle biopsies was highly correlated with poor motor outcome at a 10-year follow-up (Desguerre et al., J Neuropathol Exp Neurol 68(7):762-767, 2009). These results point to fibrosis as a major contributor to DMD muscle dysfunction and highlight the need to develop therapies that reduce fibrotic tissue.

Most anti-fibrotic therapies that have been tested in mdx mice act to block fibrotic cytokine signaling through inhibition of the TGF pathway.

MicroRNAs (miRNAs) are single-stranded RNAs of ˜22 nucleotides that mediate gene silencing at the post-transcriptional level by pairing with bases within the 3′ UTR of mRNA, inhibiting translation or promoting mRNA degradation. A seed sequence of 7 bp at the 5′ end of the miRNA targets the miRNA; additional recognition is provided by the remainder of the targeted sequence, as well as its secondary structure. MiRNAs play an important role in muscle disease pathology and exhibit expression profiles that are uniquely dependent on the type of muscular dystrophy in question (Eisenberg et al., Proc Natl Acad Sci U.S.A. 104(43):17016-17021, 2007). A growing body of evidence suggests that miRNAs are involved in the fibrotic process in many organs including heart, liver, kidney, and lung (Jiang et al., Proc Natl Acad Sci U.S.A. 104(43):17016-17021, 2007).

Recently, the down-regulation of miR-29 was shown to contribute to cardiac fibrosis (Cacchiarelli et al., Cell Metab 12(4):341-351, 2010). Reduced expression of miR-29 was genetically linked with human DMD patient muscles (Eisenberg et al., Proc Natl Acad Sci U.S.A. 104(43):17016-17021, 2007).

The miR-29 family consists of three family members expressed from two bicistronic miRNA clusters. MiR-29a is coexpressed with miR-29b (miR-29b-1); miR-29c is co-expressed with a second copy of miR-29b (miR-29b-2). The miR-29 family shares a conserved seed sequence, and miR-29a and miR-29b each differ by only one base from miR-29c. Furthermore, electroporation of miR-29 plasmid (a cluster of miR-29a and miR-29b-1) into mdx mouse muscle reduced the expression levels of ECM components, collagen and elastin, and strongly decreased collagen deposition in muscle sections within 25 days post-treatment (Cacchiarelli et al., Cell Metab 12(4):341-351, 2010).

Adeno-associated virus (AAV) is a replication-deficient parvovirus, the single-stranded DNA genome of which is about 4.7 kb in length, including 145 nucleotide inverted terminal repeat (ITRs).

AAV possesses unique features that make it attractive as a vector for delivering foreign DNA to cells, for example, in gene therapy. AAV infection of cells in culture is noncytopathic, and natural infection of humans and other animals is silent and asymptomatic. Moreover, AAV infects many mammalian cells, allowing the possibility of targeting many different tissues in vivo. Moreover, AAV transduces slowly dividing and non-dividing cells, and can persist essentially for the lifetime of those cells as a transcriptionally active nuclear episome (extrachromosomal element). The AAV proviral genome is infectious as cloned DNA in plasmids, which makes construction of recombinant genomes feasible. Furthermore, because the signals directing AAV replication, genome encapsidation and integration are contained within the ITRs of the AAV genome, some or all of the internal approximately 4.3 kb of the genome (encoding replication and structural capsid proteins, rep-cap) may be replaced with foreign DNA such as a gene cassette containing a promoter, a DNA of interest and a polyadenylation signal. The rep and cap proteins may be provided in trans. Another significant feature of AAV is that it is an extremely stable and hearty virus. It easily withstands the conditions used to inactivate adenovirus (56° to 65° C. for several hours), making cold preservation of AAV less critical. AAV may even be lyophilized. Finally, AAV-infected cells are not resistant to superinfection.

Multiple studies have demonstrated long-term (>1.5 years) recombinant AAV-mediated protein expression in muscle. See, Clark et al., Hum Gene Ther 8:659-669 (1997); Kessler et al., Proc Nat. Acad Sc. U.S.A. 93:14082-14087 (1996); and Xiao et al., J Virol 70: 8098-8108 (1996). See also, Chao et al., Mol Ther 2:619-623 (2000) and Chao et al., Mol Ther 4:217-222 (2001). Moreover, because muscle is highly vascularized, recombinant AAV transduction has resulted in the appearance of transgene products in the systemic circulation following intramuscular injection as described in Herzog et al., Proc Natl Acad Sci U.S.A. 94: 5804-5809 (1997) and Murphy et al., Proc Natl Acad Sci U.S.A. 94: 13921-13926 (1997). Moreover, Lewis et al., J Virol 76: 8769-8775 (2002) demonstrated that skeletal myofibers possess the necessary cellular factors for correct antibody glycosylation, folding, and secretion, indicating that muscle is capable of stable expression of secreted protein therapeutics.

While gene therapy using AAV vectors has fueled significant investments into the sector, significant challenges remain for commercialization. Recombinant viral vector production is seen as complex, with the production scale-up regarded as a major challenge technically, and a large barrier for commercialization.

Specifically, reported clinical doses for AAV-based viral vectors range from 10¹¹ to 10¹⁴ genomic particles (vector genomes; vg) per patient dependent on therapeutic area. Thus, from a wider gene therapy development perspective, current scale-up approaches fall short of supplying the required number of doses to allow later Phase (e.g., Phases II/III) trails to progress, thus retarding the development of gene therapy products. This is supported by the fact that the majority of clinical studies have been very small, performed on <100 patients (and in some cases <10), using adherent cell transfection processes that generate very modest amounts of product. When predicted amounts of virus required for later phase development are compared to current productivities (e.g., 5×10¹¹ vg from single 10 layer cell factory), there is real concern that this approach will fall short of the material requirements for late phase and in-market needs for even ultra-orphan diseases, which have high dose and small patient cohorts, let alone more “standard” gene therapy indications.

As is stated by Clement and Grieger in a recent review article (Molecular Therapy—Methods & Clinical Development (2016) 3, 16002; doi:10.1038/mtm.2016.2): “[t]he use of rAAV in the clinical setting has underscored the urgent need for production and purification systems capable of generating very large amounts of highly pure rAAV particles. Typical FDA-approved investigational new drug includes extensive preclinical studies for toxicology, safety, dose, and bio-distribution assessments, with vector requirements often reaching the 1E15 to 1E16 vector genome range. Manufacturing such amounts, although technically feasible, still represents an incredible effort when using the current production systems.”

This problem is particularly acute for AAV vectors that are desirably delivered systematically (as opposed to locally). In a recent article, Adamson-Small et al. (Molecular Therapy—Methods & Clinical Development (2016) 3, 16031; doi:10.1038/mtm.2016.31) stated that “[c]urrent limitations in vector production and purification have hampered widespread implementation of clinical candidate vectors, particularly when systemic administration is considered . . . . This holds specifically true for the treatment of inherited genetic diseases such as muscular dystrophies, when body-wide gene transfer may be required, relying on systemic dosing often at high AAV doses.” Indeed, previous studies of rAAV in clinical trials for muscular dystrophy have delivered vector via intramuscular injection often due to the lack of large-scale manufacturing capabilities to generate the amounts needed to support systemic administration. Systematic delivery of two AAV vectors in combination therapy poses even a greater challenge in terms of producing sufficient quantities of high quality AAV vectors required for the combination therapy.

Thus, functional improvement in patients suffering from DMD and other muscular dystrophies require both gene restoration and reduction of symptoms associated with a number of secondary cascades such as fibrosis. Alternatively or in addition, muscular dystrophies may benefit from treatments simultaneously targeting different disease-causing pathways. There is a need for methods of reducing such secondary cascade symptoms (e.g., fibrosis) that may be paired with gene restoration methods for more effective treatments of DMD and other muscular dystrophies. Such combination therapy must also overcome the significant clinical and commercialization challenge of producing sufficient quantities of gene therapy vectors to deliver both therapeutic components to the target tissue, particularly in the setting of systematic delivery of gene therapy vectors.

### SUMMARY OF THE INVENTION

The invention described herein provides a viral vector for gene therapy, comprising a polynucleotide sequence that simultaneously encodes a first polypeptide or a first RNA (“a first transcription and/or expression unit or cassette”), and a second polypeptide or a second RNA (“a second transcription and/or expression unit or cassette”) expressed from a so-called “divergent cassette” in relation to the first transcription and/or expression cassette, which confers separate and independent control for the expression of each of first and second polypeptide and/or RNA, with minimal or no transcriptional interference between different transcription and/or expression units or cassettes. Thus the viral vectors of the invention are sometimes referred to as “divergent vectors.” In certain embodiments, the two transcription and/or expression units or cassettes are each under the control of its owe independent control elements or promoters. In certain embodiments, the two independent control elements or promoters operate in opposite directions, such as each transcribing towards (as opposed to away from) its nearest terminal repeat sequences (such as the ITR sequences in an AAV vector).

As used herein, “first” and “second” transcriptional cassettes or units are relative terms, in that any one of the two transcripitonal cassettes can be called the first or the second transcripitonal cassettes. Therefore, a “first transcripitional cassette” described under one particular instance is not necessarily identical or equivalent to another “first transcripitional cassette” described under a different instance.

The invention is partly based on the surprising discovery that the one or more coding sequence(s) can be inserted into certain positions, such as into a so-called “divergent cassette” (see below) situated between the control element or promoter for the gene of interest (GOI) and the nearest ITR, while both the functional protein (such as the dystrophin microgene or minigene product) and one or more coding sequences can be expressed inside the infected target cells (e.g., muscle cells) without significant reduction in expression compared to similar vector constructs encompassing only the functional protein (such as the dystrophin minigene product) or only the one or more coding sequences. In certain embodiments, the expression of the one or more coding sequences from the divergent cassette is greatly increased compared to inserting the same coding sequences into the other parts of the viral vector, such as into the heterologous intron or 3′-UTR of the GOI.

As used herein, “transcription and/or expression unit or cassette” includes at a minimum a control element, a coding sequence, and a transcription termination sequence. The coding sequence in each transcription and/or expression unit or cassette can independently encode any of a protein, a polypeptide, an mRNA, a non-coding RNA (such as an shRNA, miRNA, siRNA, or a precursor thereof), an antisense sequence, a guide sequence for a gene editing enzyme, or a miRNA inhibitor, etc. The coding sequence is operably linked to and under the control of a control element which includes a promoter, and optionally one or more enhancers or other control elements, for initiating or affecting transcription by an RNA polymerase (including Pol II or Pol III), such that whatever that is encoded by the coding sequence can be transcribed. The coding sequence is also operably linked to a downstream transcription termination sequence (such as the T6 transcription termination sequence) so that transcription can be terminated as desired.

As used herein, constructs with two or more transcriptional units and/or cassettes operating in opposite/divergent/multiple directions are referred to as “divergent constructs.”

Specifically, viral vectors of the invention (such as an AAV-based viral vector or a lentivirus-based viral vector) having such arrangements of two transcription and/or expression units or cassettes, or divergent constructs in viral plasmid vector backbone, are referred to as “divergent vectors.”

For example, the vector of the invention may simultaneously encode a first therapeutic agent (e.g., a protein) expressed from the first transcription cassette, and a second therapeutic agent (e.g., an RNA) expressed from the divergent cassette.

It should be understood that the reference to the first and second (divergent) expression units and/or cassettes are relative, such that any of the two GOI's can be expressed from either expression units and/or cassettes. For example, in one embodiment, a microdystrophin coding sequence can be expressed from the first or the second (divergent) expression cassette. In another embodiment, any of the shRNA, siRNA, miRNA, etc., can also be expressed from the first or the second (divergent) expression cassette. In addition, both expression units/cassettes can be used to express proteins or non-protein products described herein (such as miRNA or precursors thereof).

That is, either the first or the second RNA, or both, may be a non-coding RNA that does not produce a protein or polypeptide. Such non-coding RNA can be microRNA (miR), shRNA (short hairpin RNA), piRNA, snoRNA, snRNA, exRNA, scaRNA, long ncRNAs such as Xist and HOTAIR, anti-sense RNA, or precursor thereof, preferably with therapeutic value, e.g., those associated with diseases such as cancer, autisum, Alzheimer's disease, Cartilage-hair hypoplasia, hearing loss, and Prader-Willi syndrome, particularly various types of muscular dystrophies (MDs), including DMD/BMD.

Such non-coding RNA can also be the single or multiple guide RNA(s) of a CRISPR/Cas9 protein, or a CRISPR RNA (crRNA) of a CRISPR/Cas12a(formerly Cpf1) protein.

Further, the two transcription units/cassettes may be used to express products (protein, peptide, RNA etc.) that are either biologically unrelated, or are somehow related in terms of biological function. For example, one of the coded/expressed products may replace the function of a defective gene product in a disease or condition, while the other coded/expressed product may act on a separate biological pathway and thus the two products are delivered and result in desirably additive or synergistic biological and therapeutic effects. In the context of treating muscular dystrophy, for example, one of the coded/expressed products may be a functional version of a dystrophin (such as μDys) that supplements the lost dystrophin function, while the other coded/expressed product may antagonize a side effect associated with the loss of dystrophin function, such as fibrosis.

Thus in one aspect, the invention provides a recombinant viral vector comprising: a) a first transcription cassette for expressing a first gene of interest (1st GOI) under the control of an operably linked first control element; b) a second transcription cassette for expressing a second gene of interest (2nd GOI) under the control of an operably linked second control element; wherein said first transcription cassette and said second transcription cassette do not overlap in sequence, and, wherein said first control element and said second control element transcribes the 1st GOI and the 2nd GOI, respectively, in opposite directions away from each other.

That is, the first transcription cassette and the second transcription cassette are independently under the control of their own transcription control element/promoter that directs transcription in opposite/divergent directions, preferably each towards the nearest terminal repeat sequences (e.g., ITR of AAV).

In certain embodiments, the first gene of interest encodes a wild-type or normal gene (e.g., codon optimized wild-type or normal gene) that is defective in a disease or condition, and wherein the second gene of interest encodes an antagonist that targets a product of said gene defective in the disease or condition.

In certain embodiments, the first gene of interest encodes a CRISPR/Cas enzyme (e.g., Cas9, Cas12a, Cas13a-13d), and wherein said second gene of interest encodes one or more guide RNA (e.g., sgRNA for Cas9, or crRNA for Cas12a) each specific for a target sequence.

In certain embodiments, the first gene of interest and the second gene of interest encode products that function in distinct pathways beneficial in the treatment of a disease or condition.

In certain embodiments, in the recombinant viral vector, a) the first GOI comprises a heterologous intron sequence that enhances expression of a downstream protein-coding sequence, a 3′-UTR coding region downstream of the protein-coding sequence, and the polyadenylation (polyA) signal sequence (e.g., AATAAA); b) the second GOI comprises one or more coding sequences that independently encode: a protein, a polypeptide, an RNAi sequence (siRNA, shRNA, miRNA), an antisense sequence, a guide sequence for a gene editing enzyme, a microRNA (miRNA), and/or a miRNA inhibitor; and, c) optionally, one or more additional coding sequences inserted in the heterologous intron sequence and/or in the 3′-UTR coding region of the first GOI, wherein said one or more additional coding sequences independently encode: a protein, a polypeptide, an RNAi sequence (siRNA, shRNA, miRNA), an antisense sequence, a guide sequence for a gene editing enzyme, a microRNA (miRNA), and/or a miRNA inhibitor.

In a specific embodiment, the invention provides a recombinant viral vector comprising: a) a polynucleotide encoding a functional gene or protein of interest (GOI), such as one effective to treat a muscular dystrophy, wherein said polynucleotide comprises a 3′-UTR coding region, and is immediately 3′ to a heterologous intron sequence that enhances expression of the functional protein encoded by the polynucleotide; b) a first control element (e.g., a muscle-specific control element) operably linked to and drives the expression of the polynucleotide; and, c) one or more coding sequences (1) inserted between the first control element and the nearest viral terminal sequence (e.g., ITR in AAV) and operably linked to a second control element, and (2) optionally further inserted in the intron sequence or in the 3′-UTR coding region; wherein said one or more coding sequences independently encode: a protein, a polypeptide, an RNAi sequence (siRNA, shRNA, miRNA), an antisense sequence, a guide sequence for a gene editing enzyme (such as a single guide RNA (sgRNA) for CRISPR/Cas9, or acrRNA for CRISPR/Cas12a), a microRNA (miRNA), and/or a miRNA inhibitor.

In certain embodiments, the recombinant viral vector is a recombinant AAV (adeno associated viral) vector or a recombinant lentiviral vector.

In a specific embodiment, the invention provides a recombinant AAV (rAAV) vector comprising: a) a polynucleotide encoding a functional protein effective to treat a muscular dystrophy, wherein said polynucleotide comprises a 3′-UTR coding region, and is immediately 3′ to a heterologous intron sequence that enhances expression of the functional protein encoded by the polynucleotide; b) a muscle-specific control element operably linked to and drives the expression of the polynucleotide; and, c) one or more coding sequences inserted between the muscle-specific control element and the nearest AAV ITR and operably linked to a second control element, and (2) optionally further inserted in the intron sequence or in the 3′-UTR coding region; wherein said one or more coding sequences independently encode: an RNAi sequence (siRNA, shRNA, miRNA), an antisense sequence, a microRNA (miRNA), and/or a miRNA inhibitor.

In a particular embodiment, the invention described herein provides a viral vector, such as a recombinant AAV vector, that comprises: a) a dystrophin microgene or minigene encoding a functional micro-dystrophin protein (e.g., microD5), wherein said dystrophin microgene or minigene comprises a 3′-UTR coding region, and is immediately 3′ to a heterologous intron sequence that enhances expression of the dystrophin microgene or minigene; b) a muscle-specific control element operably linked to and drives the expression of the dystrophin microgene or minigene; and, c) one or more (e.g., 1, 2, 3, 4, or 5) coding sequence(s) inserted between the muscle-specific control element and the nearest AAV ITR and operably linked to a second control element, and (2) optionally further inserted in the intron sequence or in the 3′-UTR coding region; wherein said one or more coding sequence(s) independently encode(s): an RNAi sequence (siRNA, shRNA, miRNA), an antisense sequence, a microRNA (miRNA), and/or a miRNA inhibitor.

In certain embodiments, the second control element is a promoter or portion of a promoter that transcribes the one or more coding sequences. For example, the second control element is a pol II promoter that transcribes the one or more coding sequences inserted between the first control element and the nearest viral terminal sequence, optionally in a direction opposite to the transcription initiated by the first control element. In other embodiments, the second control element is a pol III promoter. In other embodiments, the first and second control elements are both the same promoter. In other embodiments, the first and second control elements are different promoters.

In certain embodiments, the functional dystrophin protein is microD5, and/or the muscle-specific control element/promoter is CK promoter.

In certain embodiments, the one or more coding sequences are inserted into a transcription cassette that does not encode or express a functional protein. In certain embodiments, the one or more coding sequences are further inserted into the 3′-UTR coding region, or after the polyadenylation (polyA) signal sequence (e.g., AATAAA) of a transcription cassette that does encode or express a functional protein.

In certain embodiments, expression of the functional GOI is substantially unaffected in the presence of the one or more coding sequences (e.g., as compared to otherwise identical control constructs without said one or more coding sequences).

In certain embodiments, the first GOI is a wt or normal SERPINA1 coding sequence (e.g., codon optimized SERPINA1 coding sequence), and wherein the second GOI encodes an RNAi agent (e.g., siRNA, shRNA, or miRNA) that targets a mutant allele of SERPINA1.

In certain embodiments, the mutant allele of SERPINA1 is the the Pittsburgh allele, the B (Alhambra) allele, the M (Malton) allele, the S allele, the M (Heerlen) allele, the M (Mineral Springs) allele, the M (procida) allele, the M (Nichinan) allele, the I allele, the P (Lowell) allele, the null (Granite falls) allele, the null (Bellingham) allele, the null (Mattawa) allele, the null (procida) allele, the null (Hong Kong 1) allele, the null (Bolton) allele, the Pittsburgh allele, the V (Munich) allele, the Z (Augsburg) allele, the W (Bethesda) allele, the null (Devon) allele, the null (Ludwigshafen) allele, the Z (Wrexham) allele, the null (Hong Kong 2) allele, the null (Riedenburg) allele, the Kalsheker-Poller allele, the P (Duarte) allele, the null (West) allele, the S (Iiyama) allele, or the Z (Bristol) allele.

In certain embodiments, the first GOI is a codon-optimized wt or normal coding sequence for SERPINA1 having a 5′-UTR and/or a 3′-UTR different from that of the mutant SERPINA1, and wherein the RNAi agent targets a 5′-UTR target sequence, a 3′-UTR target sequence, and/or a coding sequence target sequence specifically associated with the mutant but not the codon-optimized wt allele of SERPINA1.

In certain embodiments, the first control element and/or the second control element comprises a liver specific promoter and/or enhancer, such as the ApoE enhancer and the al-antitrypsin promoter.

In certain embodiments, the first GOI is a wt or normal coding sequence for a gene defective in a repeat expansion disorder (RED) (e.g., a codon optimized wt or normal coding sequence for the gene defective in the RED), and wherein the second GOI encodes an RNAi agent (e.g., siRNA, shRNA, or miRNA) that targets a mutant allele of the gene defective in the RED.

In certain embodiments, the RED is spinocerebellar ataxia 3 (SCA3) resulting from a mutant ATXN3 gene with (more than 52) CAG trinucleotide repeats, and wherein the RNAi agent targets an SNP specifically associated with the mutant but not the wt allele of ATXN3.

In certain embodiments, the RED is spinocerebellar ataxia 3 (SCA3) resulting from a mutant ATXN3 gene with (more than 52) CAG trinucleotide repeats, wherein the first GOI is a codon-optimized wt or normal coding sequence for ATXN3 having a 5′-UTR and/or a 3′-UTR different from that of the mutant ATXN3; and wherein the RNAi agent targets a 5′-UTR target sequence, a 3′-UTR target sequence, and/or a coding sequence target sequence specifically associated with the mutant but not the codon-optimized wt allele of ATXN3.

In certain embodiments, the first control element and/or the second control element comprises a neuron specific promoter and/or enhancer (such as the synapsin promoter), or a natural ATXN3 promoter, or a ubiquitous promoter.

In certain embodiments, the RED is SCA1, 2, 3, 6, 7, 8, 10, 12, or 17, respectively, and wherein the RNAi agent targets an SNP specifically associated with the mutant but not the wt allele of ataxin-1, ataxin-2, ataxin-3, CACNA1, ataxin-7, SCA8, SCA10, PPP2R2B, or TBP, respectively.

In certain embodiments, the RED is SCA1, 2, 3, 6, 7, 8, 10, 12, or 17, respectively, wherein the first GOI is a codon-optimized wt or normal coding sequence for ataxin-1, ataxin-2, ataxin-3, CACNA1, ataxin-7, SCA8, SCA10, PPP2R2B, or TBP, respectively, having a 5′-UTR and/or a 3′-UTR different from that of the mutant ataxin-1, ataxin-2, ataxin-3, CACNA1, ataxin-7, SCA8, SCA10, PPP2R2B, or TBP, respectively; and wherein the RNAi agent targets a 5′-UTR target sequence, a 3′-UTR target sequence, and/or a coding sequence target sequence specifically associated with the mutant but not the codon-optimized wt allele of ataxin-1, ataxin-2, ataxin-3, CACNA1, ataxin-7, SCA8, SCA10, PPP2R2B, or TBP, respectively.

In certain embodiments, the RED is myotonic dystrophy type 1 (DM1) resulting from a mutant DMPK gene with (more than 50) CTG trinucleotide repeats, and wherein the RNAi agent targets an SNP specifically associated with the mutant but not the wt allele of DMPK.

In certain embodiments, the RED is myotonic dystrophy type 1 (DM1) resulting from a mutant DMPK gene with (more than 50) CTG trinucleotide repeats, wherein the first GOI is a codon-optimized wt or normal coding sequence for DMPK having a 5′-UTR and/or a 3′-UTR different from that of the mutant DMPK; and wherein the RNAi agent targets a 5′-UTR target sequence, a 3′-UTR target sequence, and/or a coding sequence target sequence specifically associated with the mutant but not the codon-optimized wt allele of DMPK.

In certain embodiments, the first control element and/or the second control element comprises a muscle specific promoter and/or enhancer (such as the CK8 promoter), or a natural DMPK promoter, or a ubiquitous promoter.

In certain embodiments, the first GOI encode a wt or codon-optimized MBNL1 gene, and wherein the second GOI encodes an RNAi agent (e.g., siRNA, shRNA, or miRNA) that targets a mutant allele of the DMPK gene defective in myotonic dystrophy type 1 (DM1) resulting from having more than 50 CTG trinucleotide repeats.

In certain embodiments, the RED is Fragile X syndrome (FXS) resulting from a mutant FMR1 gene with (more than 55) CGG trinucleotide repeats, and wherein the RNAi agent targets an SNP specifically associated with the mutant but not the wt allele of FMR1.

In certain embodiments, the RED is Fragile X syndrome (FXS) resulting from a mutant FMR1 gene with (more than 55) CGG trinucleotide repeats, wherein the first GOI is a codon-optimized wt or normal coding sequence for FMR1 having a 5′-UTR and/or a 3′-UTR different from that of the mutant FMR1; and wherein the RNAi agent targets a 5′-UTR target sequence, a 3′-UTR target sequence, and/or a coding sequence target sequence specifically associated with the mutant but not the codon-optimized wt allele of FMR1.

In certain embodiments, the first control element and/or the second control element comprises a neuron specific promoter and/or enhancer (such as the synapsin promoter), or a natural FMR1 promoter.

In certain embodiments, the first GOI encodes a functional dystrophin protein (such as microD5) under the control of a muscle-specific promoter (such as the CK8 promoter).

In certain related embodiments, in the recombinant AAV (rAAV) vector: a) the polynucleotide is a dystrophin minigene encoding a functional 5-spectrin-like repeat dystrophin protein (e.g., microD5; as described in U.S. Pat. No. 10,479,821, incorporated herein by reference); and/or, b) the muscle-specific control element is a CK promoter operably linked to and drives the expression of the dystrophin minigene.

In certain embodiments, the second GOI encodes one or more coding sequences comprise an exon-skipping antisense sequence that induces skipping of an exon of a defective dystrophin, such as any one of exons 45-55 of dystrophin, or exon 44, 45, 51, and/or 53 of dystrophin.

In certain embodiments, the microRNA is miR-1, miR-133a, miR-29c, miR-30c, and/or miR-206. For example, the microRNA may be miR-29c, optionally having a modified flanking backbone sequence that enhances the processing of the guide strand of miR-29c designed for a target sequence. In certain embodiments, the modified flanking backbone sequence is from or based on miR-30, -101, -155, or -451.

In certain embodiments, expression of the microRNA in a host cell is up-regulated by at least about 1.5-100 fold (e.g., about 2-80 fold, about 1.5-10 fold, about 15-70 fold, about 50-70 fold, about 15, 20, 25, 30, 35, 40, 45, 50, 55, 60, 65, 70, 75, or about 80 fold) compared to endogenous expression of the microRNA in the host cell.

In certain embodiments, the RNAi sequence is an shRNA against sarcolipin (shSLN).

In certain embodiments, the one or more coding sequences encode one or more identical or different shRNAs against sarcolipin (shSLN).

In certain embodiments, the shRNA reduces sarcolipin mRNA and/or sarcolipin protein expression by at least about 50%.

In certain embodiments, the GOI is CRISPR/Cas9, and the guide sequence is the sgRNA; or wherein the GOI is CRISPR/Cas12a, and the guide sequence is the crRNA.

In certain embodiments, the RNAi sequence (siRNA, shRNA, miRNA), said antisense sequence, said CRISPR/Cas9 sgRNA, said CRISPR/Cas12a crRNA and/or said microRNA antagonizes the function of one or more target genes, such as an inflammatory gene, an activator of NF-κB signaling pathway (e.g., TNF-α, IL-1, IL-1β, IL-6, Receptor activator of NF-κB (RANK), and Toll-like receptors (TLRs)), NF-κB, a downstream inflammatory cytokine induced by NF-κB, a histone deacetylase (e.g., HDAC2), TGF-β, connective tissue growth factor (CTGF), ollagens, elastin, a structural component of the extracellular matrix, Glucose-6-phosphate dehydrogenase (G6PD), myostatin, phosphodiesterase-5 (PED-5) or ACE, VEGF decoy-receptor type 1 (VEGFR-1 or Flt-1), and hematopoietic prostaglandin D synthase (HPGDS).

In certain embodiments, the heterologous intron sequence is SEQ ID NO: 1.

In certain embodiments, the vector is a recombinant AAV vector of the serotype AAV1, AAV2, AAV4, AAV5, AAV6, AAV7, AAVrh74, AAV8, AAV9, AAV10, AAV 11, AAV 12, or AAV 13.

In certain embodiments, in the vector, e.g., the recombinant AAV (rAAV) vector: a) the polynucleotide encodes a functional fukutin (FKTN) protein; and/or, b) the one or more coding sequences encode an exon-skipping antisense sequence that restores correct exon 10 splicing in a defective FKTN gene in a Fukuyama congenital muscular dystrophy (FCMD) patient.

In certain embodiments, in the vector, e.g., the recombinant AAV (rAAV) vector: a) the polynucleotide encodes a functional LAMA2 protein; and/or, b) the one or more coding sequences encode an exon-skipping antisense sequence that restores expression of the C-terminal G-domain (exons 45-64), particularly G4 and G5 of a defective LAMA2 gene in a Merosin-deficient congenital muscular dystrophy type 1A (MDC1A) patient.

In certain embodiments, in the vector, e.g., the recombinant AAV (rAAV) vector: a) the polynucleotide encodes a functional DMPK protein, or a CLCN1 gene; and/or, b) the RNAi sequence (siRNA, shRNA, miRNA), the antisense sequence, or the microRNA (miRNA) targets expanded repeats of mutant transcripts in a defective DMPK gene, or encodes an exon-skipping antisense sequence leading to the skipping of exon 7A in CLCN1 gene in a DM1 patient.

In certain embodiments, in the vector, e.g., the recombinant AAV (rAAV) vector: a) the polynucleotide encodes a functional DYSF protein; and/or, b) one or more coding sequences encode an exon-skipping antisense sequence leading to the skipping of exon 32 in a defective DYSF gene in a dysferlinopathy (LGMD2B or MM) patient.

In certain embodiments, in the vector, e.g., the recombinant AAV (rAAV) vector: a) the polynucleotide encodes a functional SGCG protein; and/or, b) one or more coding sequences encode an exon-skipping antisense sequence leading to the skipping of exons 4-7 in a defective LGMD2C gene (e.g., one with the Δ-521T SGCG mutation) in a LGMD2C patient.

In certain embodiments, one or more coding sequences are further inserted in the intron sequence.

In certain embodiments, expression of the functional protein is not negatively affected by the insertion of said one or more coding sequences.

In certain embodiments, the vector is of the serotype AAV1, AAV2, AAV4, AAV5, AAV6, AAV7, AAVrh74, AAV8, AAV9, AAV10, AAV 11, AAV 12, or AAV 13. In certain embodiments, the vector is a derivative of a known serotype. In certain embodiments, the derivative may exhibit a desired tissue specificity or tropism, a desired immunogenic profile (e.g., not subject to attack by a subject patient's immune system), or other desirable properties for a pharmaceutical composition or gene therapy for various indications.

In certain embodiments, the first control element (or the promoter in the divergent cassette) is a promoter or portion of a promoter that transcribes in a tissue specific manner the one or more coding GOI sequences. In certain embodiments, the tissue specific specific control element is a muscle-specific control element.

In certain embodiments, the muscle-specific control element is human skeletal actin gene element, cardiac actin gene element, myocyte-specific enhancer binding factor mef, muscle creatine kinase (MCK), truncated MCK (tMCK), myosin heavy chain (MHC), C5-12, murine creatine kinase enhancer element, skeletal fast-twitch troponin c gene element, slow-twitch cardiac troponin c gene element, slow-twitch troponin i gene element, hypoxia-inducible nuclear factors, steroid-inducible element, or glucocorticoid response element (gre).

In certain embodiments, the muscle-specific control element comprises the nucleotide sequence of SEQ ID NO: 10 or SEQ ID NO: 11 of WO2017/181015 (incorporated herein by reference).

Another aspect of the invention provides a composition comprising any of the vector, e.g., the recombinant viral (AAV) vector of the invention.

In certain embodiments, the composition is a pharmaceutical composition further comprising a therapeutically compatible carrier, diluent, or excipient.

In certain embodiments, the therapeutically acceptable carrier, diluent, or excipient is a sterile aqueous solution comprising 10 mM L-histidine at pH 6.0, 150 mM sodium chloride, and 1 mM magnesium chloride.

In certain embodiments, the composition is in a dosage form of about 10 mL of aqueous solution having at least 1.6×10¹³ vector genomes.

In certain embodiments, the composition has a potency of at least 2×10¹² vector genomes per milliliter.

Another aspect of the invention provides a method of producing the subject composition, comprising producing the vector, e.g., the recombinant AAV vector in a cell and lysing the cell to obtain the vector.

In certain embodiments, the vector is an AAV1, AAV2, AAV4, AAV5, AAV6, AAV7, AAVrh74, AAV8, AAV9, AAV10, AAV 11, AAV 12, or AAV 13 vector.

Another aspect of the invention provides a method of treating a muscular dystrophy or dystrophinopathy in a subject in need thereof, the method comprising administering to the subject a therapeutically effective amount of any one of the recombinant vector, e.g., the recombinant AAV vector of the invention, or any one of the composition of the invention.

In certain embodiments, the muscular dystrophy is Duchenne muscular dystrophy or Becker muscular dystrophy.

In certain embodiments, the muscular dystrophy is Duchenne muscular dystrophy, Becker muscular dystrophy, Fukuyama congenital muscular dystrophy (FCMD), dysferlinopathy, myotonic dystrophy, and merosin-deficient congenital muscular dystrophy type 1A, facioscapulohumeral muscular dystrophy (FSHD), congenital muscular dystrophy (CMD), or limb-girdle muscular dystrophy (LGMDR5 or LGMD2C).

Another aspect of the invention provides a method of treating Alpha-1 antitrypsin deficiency (AATD) in a subject in need thereof, the method comprising administering to the subject a therapeutically effective amount of the recombinant viral vector (e.g., the recombinant AAV vector) of the invention, or the composition comprising such recombinant viral vector.

Another aspect of the invention provides a method of treating spinocerebellar ataxia 3 (SCA3) in a subject in need thereof, the method comprising administering to the subject a therapeutically effective amount of the recombinant viral vector (e.g., the recombinant AAV vector) of the invention, or the composition comprising such recombinant viral vector.

Another aspect of the invention provides a method of treating myotonic dystrophy type 1 (DM1) in a subject in need thereof, the method comprising administering to the subject a therapeutically effective amount of the recombinant viral vector (e.g., the recombinant AAV vector) of the invention, or the composition comprising such recombinant viral vector.

Another aspect of the invention provides a method of treating Fragile X syndrome (FXS) in a subject in need thereof, the method comprising administering to the subject a therapeutically effective amount of the recombinant viral vector (e.g., the recombinant AAV vector) of the invention, or the composition comprising such recombinant viral vector.

In certain embodiments, the recombinant vector, e.g., the recombinant AAV vector or the composition is administered by intramuscular injection, intravenous injection, parental administration or systemic administration.

Another aspect of the invention provides a kit for preventing or treating a disease, such as DMD or related/associated diseases, in a subject, the kit comprising: one or more vector, e.g., the recombinant AAV as described herein, or a composition as described herein; instructions for use (written, printed, electronic/optical storage media, or online); and/or packaging. In certain embodiments, a kit also includes a known therapeutic composition for treating the disease (e.g., DMD), for combination therapy.

It should be understood that any one embodiment described herein, including one described only in the example or claims, can be combined with any one or more other embodiments of the invention unless such combination is expressly disclaimed or improper.

### BRIEF DESCRIPTION OF THE DRAWINGS

FIG. 1 shows schematic drawings (not to scale) showing a representative and non-limiting embodiment of the subject recombinant viral (e.g., lentiviral or AAV) vector encompassing a first gene of interest (GOI) and a second GOI, each expressed from a separate transcription cassette, under the control of separate transcriptional control elements, but in different orientation/direction and away from each other, wherein the transcription cassettes do not overlap in sequence. Such constructs can be used to express any two or more GOIs, the products of which may co-operate, preferably synergistically, to achieve a desired biological outcome. For example, one of the GOIs may encode a microdystrophin, minidystrophin or dystrophin minigene as described below (e.g., the 5-spectrin-like-repeat microD5 dystrophin protein described below, or a version of a functional DMD gene (micro dystrophin or labeled as “μDys” in the figure)), and another GOI may encode one or more additional coding sequences that can be any one of a protein, a polypeptide, or a non-protein coding RNA (ncRNA) such as shRNA. The coding sequence for the RNAi, miRNA, etc. can be inserted anywhere in the vector where “Transcript” is indicated, e.g., in the region between the promoter for one of the GOIs (labeled in the figure as the exemplary muscle specific promoter CK8) and the nearest ITR sequence; in the intron before the GOI; in the 3′-UTR region; or after the polyA signal sequence. The additional ncRNA (e.g., shRNA) coding sequences can be the same or different. These so-called “divergent/independent” transcripts of the invention are transcribed from their respective own, independent promoters, and are described in further details below.

FIG. 2 shows the relative miR-29c expression level changes (in folds over the control vector expressing μDys only) in human iPS-derived cardiomyocytes, for the various recombinant viral (e.g., AAV) vectors encoding miR-29c, either as the sole coding sequence in the viral vector (the “Solo” constructs), as part of the fusion constructs described in PCT/US2019/065718, filed on Dec. 11, 2019 (the “Fusion” constructs), or as part of the divergent constructs of the present disclosure (the “Divergent” constructs).

FIG. 3 shows relative expression levels of miR-29c in differentiated C2C12 myotube or primary mouse cardiomyocytes for the various recombinant AAV vectors encoding miR-29c, either as the sole coding sequence in the viral vector (the “Solo” constructs), or as part of the divergent constructs of the present disclosure (the “Divergent” constructs).

FIG. 4 shows about 90% knock-down of mouse SLN luciferase construct levels measured via firefly activity normalized to control renilla construct activity via multiple shSLN-μDys divergent construct of the present disclosure. As comparison, results from using solo constructs were also shown. The last two samples are commercial negative and positive shRNA controls for mSLN knock-down.

FIG. 5 shows relative expression levels of siSLN (processed siRNA product from the transcribed shSLN) in differentiated C2C12 myotubes or mouse cardiomyocytes for the various recombinant AAV vectors encoding shSLN, either as the sole coding sequence in the viral vector (“Solo”), or as part of the divergent construct of the present disclosure (“Divergent”).

FIG. 6 shows up to ˜90-95% human SLN mRNA knock-down in human iPS-derived cardiomyocytes by several subject divergent constructs encoding shhSLN.

FIG. 7 shows up to ˜90% mouse SLN mRNA knock-down in primary mouse cardiomyocytes by several subject solo and divergent constructs encoding shmSLN.

FIG. 8 shows normalized μDys mRNA levels of several Hum-shSLN-μDys divergent constructs in human iPS-derived cardiomyocytes.

FIG. 9 is an image of denaturing agarose gel, suggesting largely intact AAV genomes in the solo, fusion, and divergent constructs with miR-29c or shSLN coding sequence.

FIG. 10 shows that the ratio of all three AAV9 capsid proteins VP1-VP3 remains the same across AAV9-based solo, fusion, and divergent vectors.

FIG. 11 shows up to 6-fold miR-29c up-regulation in left gastrocnemius (top panel), up to 5.8-fold miR-29c up-regulation in diaphragm (lower left panel), and up to 7.5-fold miR-29c up-regulation in left ventricle (lower right panel), using a miR-29c-μDys divergent construct of the invention in AAV9 vector.

FIG. 12 shows elevated plasma levels of miR-29c in mice infected by the solo or divergent vectors.

FIG. 13 shows no reduction of μDys expression at RNA (left panel) and protein (right panel) level in left gastrocnemius with miR-29c up-regulating divergent AAV9 vector.

FIG. 14 shows up to 75% mSLN mRNA down-regulation in the diaphragm (top panel), up to 95% mSLN mRNA down-regulation in the left atrium (lower left panel), and up to 80% mSLN mRNA down-regulation in the left gas (lower right panel), via AAV9-mediated expression of shSLN-μDys divergent construct relative to μDys-only AAV9.

FIG. 15 shows similar levels of μDys RNA/protein expression in diaphragm via an shmSLN-μDys divergent construct of AAV9. Similar results were also obtained for atrium (data not shown).

FIG. 16 shows similar levels of μDys protein expression and mSLN mRNA knock down in tongue, via an shmSLN-μDys divergent construct of AAV9.

FIG. 17 shows that miR-29c solo and miR-29c-μDys divergent constructs of AAV9 reduce serum CK levels. Mir-29c and μDys co-expression may cause further reductions in serum CK levels.

FIG. 18 shows serum CK levels in shmSLN solo and divergent constructs of AAV9.

FIG. 19 shows that miR-29c solo and miR-29c-μDys divergent constructs of AAV9 reduce serum TIMP1 levels.

FIG. 20 shows largely similar biodistribution of miR-29c or shSLN vectors in gastrocnemius from several miR-29c-μDys divergent vectors of AAV9, or shmSLN-μDys divergent vectors of AAV9.

FIG. 21 shows generally lower titers of AAV9 vectors in liver for miR-29c-μDys and shmSLN-μDys divergent constructs vs. the μDys solo construct.

FIG. 22 shows that plasma ALT levels were comparable in animals infected by miR-29c-expressing divergent vectors, suggesting that liver damage was not a likely cause for observed lower liver titer in certain infected animals.

FIG. 23 shows added benefit of the divergent constructs of the invention over μDys construct alone in diaphragm, based on their effects on two fibrotic marker genes.

### DETAILED DESCRIPTION OF THE INVENTION

Without a parallel approach to treat a varieties of secondary cascade symptoms such as fibrosis and abnormal elevation of intracellular Ca²⁺, it is unlikely that the benefits of exon skipping, stop-codon read-through, or gene replacement therapies can ever be fully achieved. Even small molecules or protein replacement strategies are likely to fail without an approach to reduce symptoms of such secondary cascade events including muscle fibrosis. For example, previous work in aged mdx mice with existing fibrosis treated with AAV micro-dystrophin demonstrated that one could not achieve full functional restoration (Human molecular genetics 22:4929-4937, 2013). It is also known that progression of DMD cardiomyopathy is accompanied by scarring and fibrosis in the ventricular wall.

The present invention is partly directed to gene therapy methods to treat a patient that not only compensate defects in dystrophin and its function by providing a replacement, functional dystrophin minigene, but also directly target one or more secondary cascade genes using one or more additional coding sequences in the same gene therapy vector, thus achieving combination therapy in one compact vector for systematic delivery.

Indeed, the present invention, particularly the recombinant AAV (rAAV) vector of the invention, is not limited to treating DMD. The invention is applicable for treating other muscular dystrophies in which a gene is defective. For example, the recombinant AAV (rAAV) vector of the invention can provide a functional protein and/or one or more coding sequences (such as non-coding RNAs, e.g., RNAi sequence, antisense RNA, miRNA) to treat the muscular dystrophy, wherein the functional protein either provides a wild-type substitute for the defective gene product in the muscular dystrophy, or provides a non-wild-type substitute that is nevertheless effective to treat the muscular dystrophy (e.g., the 5-spectrin-like microD5 dystrophin minigene product).

Further, the present invention, particularly the recombinant AAV (rAAV) vector of the invention, is also not limited to treating muscular dystrophies. It can be used to express at least two genes of interest (GOI1 and GOI2), each appears to be able to subject to independent transcriptional control without regard to the presence or absence of the other transcriptional cassettes, and the expression level of the GOIs appears to be higher, sometimes unexpected much higher, than the previously described fusion constructs in PCT/US2019/065718, filed on Dec. 11, 2019.

Thus in one aspect, the invention provides a recombinant viral vector, e.g., a recombinant lentiviral or AAV (rAAV) vector, comprising: a) a first transcription cassette for expressing a first gene of interest (1st GOI) under the control of an operably linked first control element; b) a second transcription cassette for expressing a second gene of interest (2nd GOI) under the control of an operably linked second control element; wherein said first transcription cassette and said second transcription cassette do not overlap in sequence (except for transcription control elements such as promoters and/or enhancers), and, wherein said first control element and said second control element transcribes the 1st GOI and the 2nd GOI, respectively, in opposite directions away from each other.

As used herein, each GOI encodes at least one gene product. The gene product may be a protein or a peptide, as well as a functional RNA that may not be ultimately translated into a protein or polypeptide, such as an RNAi agent (e.g., shRNA, siRNA, miRNA), a regulatory RNA, or an antisense sequence etc. Initially transcribed RNA gene product may be further processed intracellularly to yield functional forms.

In addition, each GOI may encode more than one gene product. For example, in any of the transcription cassettes, a protein-coding sequence may contain an intron and/or UTR regions (such as 3′UTR region). Coding sequences for certain non-coding RNA gene products can be inserted into or embedded within one of the transcription cassettes, such as inside the intron or the 3′UTR region. Upon transcription of the initial RNA product from the transcription cassette, a mature mRNA encoding a protein product, as well as one or more non-coding RNA gene products will result after intracellular RNA processing.

In certain embodiments, additional GOI(s) (e.g., 3rd GOI) may be present on the same viral vector, e.g., between the first and the second transcription cassettes, or (completely or partially) overlaps with the first and the second transcription cassettes. Such additional GOIs may be operably linked to the transcription control elements of the first or the second transcription cassettes, or be under the control of their own transcriptional control elements.

As used herein, “in opposite directions and away from each other” means that the template strands (used by RNA polymerase as template for transcription) in the first and second transcription cassettes reside on different strands of the double-stranded vector DNA (i.e., the template strand for the first transcription cassette is on one strand, and the template strand for the second transcription cassette is on the other/complementary strand). Further, transcription from the promoter of the first transcription cassette directs the RNA polymerase to move further away from (rather than towards) the promoter of the second transcription cassette, and vice versa.

In certain embodiments, the two non-overlapping transcription cassettes are separated from each other by 0, 1, 5, 10, 20, 50, 100, 150, or 200 nucleotides.

In certain embodiments, the two non-overlapping transcription cassettes are separated from each other by about 20-30 nucleotides.

In certain embodiments, an insulator sequence (e.g., CTCF binding site) is inserted between promoters of the different transcription cassettes so as to minimize interaction of adjacent promoters, and/or enhance target-specific expression of each transcription unit. In vertebrates, the enhancer blocking activity of insulator sequences is associated with a binding site for the CCCTC-binding factor (CTCF). CTCF is a ubiquitously expressed nuclear protein/evolutionary conserved transcription factor with 11 zinc finger DNA-binding domains. It recognizes long and diverse nucleotide sequences, and is involved in various aspects of gene regulation.

One advantage of the viral vector of the invention is that the design and construction of the subject vector, as well as its delivery to a target cell or tissue, offer more opportunities for customization and/or optimization to fit specific biological needs. This is partly due to the fact that the expression of the multiple encoded GOIs can be independently and separately controlled at multiple levels.

For example, each GOI in the vector can carry its own transcription control elements, such as separate promoters and enhancers. In certain embodiments, the promoters and/or the enhancers for the different transcription cassettes can be identical. In certain other embodiments, the promoters and/or the enhancers for the different transcription cassettes can be different. In the latter case, depending on the tissue or cell the vector is in, maybe only one promoter/enhancer is activated, or maybe the different promoter/enhancer is activated to different extent. Specifically, in certain embodiments, one promoter/enhancer may be a tissue specific one, while another promoter/enhancer may be a ubiquitous one. In certain embodiments, one promoter/enhancer may be an inducible one, while another promoter/enhancer may be a constitutive one, or a different inducible one.

Examples of tissue specific promoters and inducible promoters are known in the art, including any described herein below.

In certain embodiments, for AAV viral vectors, AAV tropism based on the natural or engineered viral capsid proteins is selected in order to preferentially target the expression of any GOIs in selected/desired tissues or organs.

In certain embodiments, the viral vector of the invention can be locally (as opposed to systemically) delivered to a chosen organ, tissue, or cell type to maximize the delivery of limited quantities of viral vectors to desired target sites, and/or to avoid undesirable side effects.

Another distinct advantage of the subject vector is that it can take into consideration of the existence of any biological feedback loops in any given biological system. For example, in certain circumstances, modulating the activity of one target gene may off set the balance of an existing biological feedback loop in a given system, and may either lead to an undesirable side effect, or present another opportunity for further intervention. The existence of a second GOI under an independent expression control, the level of which can be much higher than previously achievable, offers a unique opportunity to either counter act the undesirable side effect, or deliver an additive if not synergistic treatment option.

Partly due to the many advantageous discussed herein, the divergent vectors of the invention can be employed in a wide range of applications.

As described in more details below, in certain genetic disorders, an endogenous gene becomes defective or dysfunctional, such that the normal function of that gene is lost and needs to be replaced or supplemented. Meanwhile the defective/dysfunctional gene encodes a mutant protein that is itself defective or dysfunctional or otherwise contributes to the cause of the pathological condition. Under such circumstances, it may not be sufficient to simply supply the lost normal function of the wild-type gene. Rather, it may also be necessary to remove or at least reduce the deleterious effect of the mutant protein.

Thus, in certain embodiments, the first gene of interest may encodes a wild-type (wt) or normal gene (e.g., codon optimized wild-type or normal gene) that is defective in a disease or condition, and wherein said second gene of interest may encodes an antagonist that targets a (mutant) product of said gene defective in the disease or condition.

For example, in certain embodiments, the first GOI is a wt or normal SERPINA1 coding sequence (e.g., codon optimized SERPINA1 coding sequence), and wherein the second GOI encodes an RNAi agent (e.g., siRNA, shRNA, or miRNA) that targets a mutant allele of SERPINA1.

In certain embodiments, the mutant allele of SERPINA1 is the the Pittsburgh allele, the B (Alhambra) allele, the M (Malton) allele, the S allele, the M (Heerlen) allele, the M (Mineral Springs) allele, the M (procida) allele, the M (Nichinan) allele, the I allele, the P (Lowell) allele, the null (Granite falls) allele, the null (Bellingham) allele, the null (Mattawa) allele, the null (procida) allele, the null (Hong Kong 1) allele, the null (Bolton) allele, the Pittsburgh allele, the V (Munich) allele, the Z (Augsburg) allele, the W (Bethesda) allele, the null (Devon) allele, the null (Ludwigshafen) allele, the Z (Wrexham) allele, the null (Hong Kong 2) allele, the null (Riedenburg) allele, the Kalsheker-Poller allele, the P (Duarte) allele, the null (West) allele, the S (Iiyama) allele, or the Z (Bristol) allele.

In certain embodiments, the first GOI is a codon-optimized wt or normal coding sequence for SERPINA1 having a 5′-UTR and/or a 3′-UTR different from that of the mutant SERPINA1, and wherein the RNAi agent targets a 5′-UTR target sequence, a 3′-UTR target sequence, and/or a coding sequence target sequence specifically associated with the mutant but not the codon-optimized wt allele of SERPINA1.

In certain embodiments, the first control element and/or the second control element comprises a liver specific promoter and/or enhancer, such as the ApoE enhancer and the al-antitrypsin promoter.

In certain embodiments, the first GOI is a wt or normal coding sequence for a gene defective in a repeat expansion disorder (RED) (e.g., a codon optimized wt or normal coding sequence for the gene defective in the RED), and wherein the second GOI encodes an RNAi agent (e.g., siRNA, shRNA, or miRNA) that targets a mutant allele of the gene defective in the RED.

In certain embodiments, the RED is spinocerebellar ataxia 3 (SCA3) resulting from a mutant ATXN3 gene with (more than 52) CAG trinucleotide repeats, and wherein the RNAi agent targets an SNP specifically associated with the mutant but not the wt allele of ATXN3.

In certain embodiments, the RED is spinocerebellar ataxia 3 (SCA3) resulting from a mutant ATXN3 gene with (more than 52) CAG trinucleotide repeats, wherein the first GOI is a codon-optimized wt or normal coding sequence for ATXN3 having a 5′-UTR and/or a 3′-UTR different from that of the mutant ATXN3; and wherein the RNAi agent targets a 5′-UTR target sequence, a 3′-UTR target sequence, and/or a coding sequence target sequence specifically associated with the mutant but not the codon-optimized wt allele of ATXN3.

In certain embodiments, the first control element and/or the second control element comprises a neuron specific promoter and/or enhancer (such as the synapsin promoter), or a natural ATXN3 promoter.

In certain embodiments, the RED is SCA1, 2, 3, 6, 7, 8, 10, 12, or 17, respectively, and wherein the RNAi agent targets an SNP specifically associated with the mutant but not the wt allele of ataxin-1, ataxin-2, ataxin-3, CACNA1, ataxin-7, SCA8, SCA10, PPP2R2B, or TBP, respectively.

In certain embodiments, the RED is SCA1, 2, 3, 6, 7, 8, 10, 12, or 17, respectively, wherein the first GOI is a codon-optimized wt or normal coding sequence for ataxin-1, ataxin-2, ataxin-3, CACNA1, ataxin-7, SCA8, SCA10, PPP2R2B, or TBP, respectively, having a 5′-UTR and/or a 3′-UTR different from that of the mutant ataxin-1, ataxin-2, ataxin-3, CACNA1, ataxin-7, SCA8, SCA10, PPP2R2B, or TBP, respectively; and wherein the RNAi agent targets a 5′-UTR target sequence, a 3′-UTR target sequence, and/or a coding sequence target sequence specifically associated with the mutant but not the codon-optimized wt allele of ataxin-1, ataxin-2, ataxin-3, CACNA1, ataxin-7, SCA8, SCA10, PPP2R2B, or TBP, respectively.

In certain embodiments, the RED is myotonic dystrophy type 1 (DM1) resulting from a mutant DMPK gene with (more than 50) CTG trinucleotide repeats, and wherein the RNAi agent targets an SNP specifically associated with the mutant but not the wt allele of DMPK.

In certain embodiments, the RED is myotonic dystrophy type 1 (DM1) resulting from a mutant DMPK gene with (more than 50) CTG trinucleotide repeats, wherein the first GOI is a codon-optimized wt or normal coding sequence for DMPK having a 5′-UTR and/or a 3′-UTR different from that of the mutant DMPK; and wherein the RNAi agent targets a 5′-UTR target sequence, a 3′-UTR target sequence, and/or a coding sequence target sequence specifically associated with the mutant but not the codon-optimized wt allele of DMPK.

In certain embodiments, the first control element and/or the second control element comprises a muscle specific promoter and/or enhancer (such as the CK8 promoter), or a natural DMPK promoter, or a ubiquitous promoter.

In certain embodiments, the first GOI encode a wt or codon-optimized MBNL1 gene, and wherein the second GOI encodes an RNAi agent (e.g., siRNA, shRNA, or miRNA) that targets a mutant allele of the DMPK gene defective in myotonic dystrophy type 1 (DM1) resulting from having more than 50 CTG trinucleotide repeats.

In certain embodiments, the RED is Fragile X syndrome (FXS) resulting from a mutant FMR1 gene with (more than 55) CGG trinucleotide repeats, and wherein the RNAi agent targets an SNP specifically associated with the mutant but not the wt allele of FMR1.

In certain embodiments, the RED is Fragile X syndrome (FXS) resulting from a mutant FMR1 gene with (more than 55) CGG trinucleotide repeats, wherein the first GOI is a codon-optimized wt or normal coding sequence for FMR1 having a 5′-UTR and/or a 3′-UTR different from that of the mutant FMR1; and wherein the RNAi agent targets a 5′-UTR target sequence, a 3′-UTR target sequence, and/or a coding sequence target sequence specifically associated with the mutant but not the codon-optimized wt allele of FMR1.

In certain embodiments, the first control element and/or the second control element comprises a neuron specific promoter and/or enhancer (such as the synapsin promoter), or a natural FMR1 promoter.

In certain embodiments, the first GOI encodes a functional dystrophin protein (such as microD5) under the control of a muscle-specific promoter (such as the CK8 promoter).

Also as described in more details below, in certain genetic disorders, effective treatment may be enhanced by simultaneously targeting two genes using just one vector, especially when the first gene of interest and the second gene of interest encode products that function in distinct pathways, yet both benefit the treatment of the disease or condition.

In yet another embodiment, the vector of the invention can be used to deliver CRISPR/Cas systems to a target cell for gene editing, or any use based on CRISPR/Cas. Specifically, one of the GOI may encode a Cas enzyme, such as Cas9, Cas12a, Cas13a-13d. Meanwhile, the other GOI may encoded one or more guide RNA matching the encoded Cas enzyme, such as the sgRNA for Cas9, or the crRNA for Cas12a.

Each of the above selected embodiments that can be carried out using the subject vectors has been described further and exemplified below.

For example, a specific divergent vector of the invention may comprise: a) the first GOI comprising a heterologous intron sequence that enhances expression of a downstream protein-coding sequence, a 3′-UTR coding region downstream of the protein-coding sequence, and the polyadenylation (polyA) signal sequence (e.g., AATAAA); b) the second GOI comprising one or more coding sequences that independently encode: a protein, a polypeptide, an RNAi sequence (siRNA, shRNA, miRNA), an antisense sequence, a guide sequence for a gene editing enzyme, a microRNA (miRNA), and/or a miRNA inhibitor; and, c) optionally, one or more additional coding sequences inserted in the heterologous intron sequence and/or in the 3′-UTR coding region of the first GOI, wherein the one or more additional coding sequences independently encode: a protein, a polypeptide, an RNAi sequence (siRNA, shRNA, miRNA), an antisense sequence, a guide sequence for a gene editing enzyme, a microRNA (miRNA), and/or a miRNA inhibitor.

In certain embodiments, expression of the first GOI and/or the second GOI is substantially unaffected in the presence of each other.

In a related embodiment, another specific divergent vector of the invention can be used as a viral vector for simultaneously delivering/expressing two or more components of an enzyme-based gene editing system, e.g., such as a target sequence-specific (engineered) nuclease that can create DNA double stranded break (DSB) at a target genomic site/target genomic sequence, and a donor or template sequence that matches the (wild-type or desired) target genome sequence. Such a system makes it possible to utilize the endogenous homologous recombination (HR) processes within the target cell to edit out a defective/undesired target genomic sequence, and replace it with a wild-type or otherwise desired sequence at the desired target genomic location.

In certain embodiments, the recombinant viral vector is a recombinant AAV (adeno associated viral) vector.

For example, the target sequence-specific (engineered) nuclease may include meganucleases (such as those in the LAGLIDADG family) and variants thereof that recognize unique target genomic sequences; Zinc Finger Nucleases (ZFNs); Transcription Activator-Like Effector Nucleases (TALENs); and CRISPR/Cas gene editing enzymes.

In the case of CRISPR/Cas, for example, the subject vector can simultaneously deliver, other than or in addition to the donor sequence, one or more gene editing guide sequence(s) having a desired sequence(s) for targeting one or more target sequence(s), and a compatible editing enzyme that can be encoded by the viral vector as the GOI. Such a viral delivery system can be used to substitute the undesired sequence occurring in the cell, tissue, or organism for the desired sequence. One example of the CRISPR/Cas enzyme system is CRISPR/Cas9 or CRISPR/Cas12a (formerly Cpf1), and one or more required guide sequences (e.g., single guide RNA or sgRNA for Cas9, or crRNA for Cas12a) to a target cell. Cas9 includes the wild-type Cas9 and functional variants thereof. Several Cas9 variants are about the same size as the micro Dystrophin gene, and can be the functional GOI encoded by the viral vector of the invention. Cas12a is even smaller than Cas9 and can also be encoded as the GOI. In certain embodiments, the Cas genes encoded by the viral constructs may or may not have UTR and/or intron elements.

In certain embodiments, the GOI is CRISPR/Cas9, and the guide sequence is an sgRNA (single guide RNA); or wherein the GOI is CRISPR/Cas12a, and the guide sequence is a crRNA.

In certain embodiments, the recombinant viral vector is a recombinant AAV (adeno associated viral) vector.

In certain embodiments, the recombinant viral vector is a lentiviral vector.

Thus in a related aspect, the invention provides a recombinant lentiviral vector for use in ex vivo or in vivo gene therapy. In ex vivo gene therapy, cultured host cells are transfected in vitro using a subject viral vector to express the gene of interest, and then transplanted into the body. In vivo gene therapy is a direct method of inserting the genetic material into the targeted tissue, and transduction takes place within the patient's own cells.

In certain embodiment, the lentiviral vector of the invention may comprise: a) a polynucleotide encoding a functional gene or protein (GOI) effective to treat the muscular dystrophy in a patient/subject/individual in need of treatment, wherein said polynucleotide comprises a 3′-UTR coding region, and is immediately 3′ to a heterologous intron sequence that enhances expression of the functional protein encoded by the polynucleotide, wherein the corresponding wild-type of the functional protein is defective in a muscular dystrophy, or wherein the functional protein, though not wild-type, is nevertheless effective to treat the muscular dystrophy; b) a first control element (e.g., a muscle-specific control element) operably linked to and drives the expression of the polynucleotide; and, c) one or more coding sequences (1) inserted between the first control element and the nearest viral terminal sequence (e.g., ITR in AAV) and operably linked to a second control element, and (2) optionally further inserted in the intron sequence or in the 3′-UTR coding region or else wherein the expression cassette; wherein said one or more coding sequences independently encode: an RNAi sequence (siRNA, shRNA, miRNA), an antisense sequence, a microRNA (miRNA), and/or a miRNA inhibitor.

As used herein, and depending on context, the term “fusion” may have different meanings, including fusion proteins, fusion RNA transcripts in which more than one encoded sequence may be present (such as the coding sequence for the GOI and the coding sequence for one or more RNAi agents etc inserted into/embedded in the 3-UTR region or intron sequences of the GOI, and fusion constructs in which the viral vectors contain coding sequences for the GOI and the one or more RNAi agents, etc.

Again, as used herein, and depending on context, the term “divergent” (e.g., as used in “divergent construct” or “divergent (viral) vector” etc) refers to the fact that there are at least two transcription cassettes or units in the viral construct/vector, such that one (under the control of the first control element or first promoter) is responsible for the transcription of one (first) GOI (protein, polypeptide, RNA, etc) in the first transcription cassette/unit, while the other (second) GOI (under the control of the second control element or second promoter) is responsible for the transcription of other encoded sequences other than the first GOI, such as the ncRNA or another protein-coding sequence, wherein the two transcription cassettes largely operate separately and independently from each other. The second transcription cassettes/units may be situated between the promoter for the first transcription unit for the first GOI, and the nearest viral vector terminal sequence (such as the nearest ITR in the AAV vector). In certain embodiments, the second transcription unit transcribes in the opposite direction compared to the transcription direction of the first transcription units, and away from each other.

In certain embodiments, the second control element is a promoter or portion of a promoter that transcribes the one or more coding sequences. For example, the second control element is a pol II promoter that transcribes the one or more coding sequences inserted between the first control element and the nearest viral terminal sequence, in a direction opposite to the transcription initiated by the first control element. In other embodiments, the second control element is a pol III promoter. In other embodiments, the first and second control elements are both the same promoter. In other embodiments, the first and second control elements are different promoters.

In certain embodiments, expression of one GOI is up- or down-regulated due to the presence of the other GOI (e.g., as compared to otherwise identical control constructs without the other GOI).

In certain embodiments, expression of one GOI is substantially unaffected in the presence of the other GOI.

For example, in certain embodiments, an insulator sequence (e.g., CTCF binding site) is inserted between promoters of the different transcription cassettes so as to minimize interaction of adjacent promoters, and/or enhance target-specific expression of each transcription unit.

### Treatment of Muscular Dystrophy

In certain embodiments, the divergent vectors of the invention can be used to treat muscular dystrophy, in that one GOI encodes a gene defective in muscular dystrophy.

As used herein, “muscular dystrophy (MD)” includes a group of diseases characterized by progressive weakness and loss of muscle mass, due to abnormal genes or gene mutations that interfere with the production of wild-type proteins needed to form healthy muscle. MD includes Duchenne muscular dystrophy (DMD); Becker muscular dystrophy (BMD); a congenital muscular dystrophy (CMD), particularly one with an identified genetic mutation, such as the ones described hereinbelow, including Fukuyama congenital muscular dystrophy (FCMD) and Merosin-deficient congenital muscular dystrophy type 1A (MDC1A); dysferlinopathy (LGMD2B and Miyoshi myopathy); myotonic dystrophy; limb-girdle muscular dystrophy (LGMD) such as LGMD2C; and Facioscapulohumeral (FSHD).

As used herein, “patient,” “subject,” and “individual” are used interchangeably to include a mammalian (e.g., human) subject to be treated, diagnosed, and/or to obtain a biological sample from in the subject methods. Typically, the subject is affected or likely to be affected with DMD and the other related diseases described herein, and in some embodiments, DMD and associated cardiomyopathy and dystrophic cardiomyopathy. In a particular embodiment, a subject is a human child or adolescent (e.g., no more than 18 years old, 15 years old, 12 years old, 10 years old, 8 years old, 5 years old, 3 years old, 1 year old, 6 months old, 3 months old, 1 month old, etc.). In a particular embodiment, the child or adolescent is male. In another particular embodiment, a subject is a human adult (e.g., >18 years old), such as a male adult.

The full-length dystrophin gene is 2.6 mb and encodes 79 exons. The 11.5-kb coding sequence translates into a 427-kD protein. Dystrophin can be divided into four major domains, including the N-terminal domain, rod domain, cysteine-rich domain, and C-terminal domain. The rod domain can be further divided into 24 spectrin-like repeats and four hinges.

A functional “dystrophin minigene” or “dystrophin microgene” has less than 24 spectrin-like repeats and one or more hinge region/s compatible with gene therapy delivery vectors (adenoviral and lentiviral) and have been described in U.S. Pat. Nos. 7,001,761, 6,869,777, 8,501,920, 7,892,824, U.S. Ser. No. 10/479,821, and U.S. Ser. No. 10/166,272 (all incorporated herein by reference).

In one embodiment, the muscular dystrophy is DMD or BMD, and in the recombinant AAV (rAAV) vector: a) the polynucleotide is a dystrophin minigene encoding a functional 5-spectrin-like repeat dystrophin protein (such as the microD5 dystrophin protein as described in U.S. Pat. No. 10,479,821, incorporated herein by reference); and/or, b) the muscle-specific control element is a CK promoter operably linked to and drives the expression of the dystrophin minigene.

As used herein, “microD5,” “microdystrophin minigene encoded by SGT-001,” or “SGT-001” for short, refers to a specific engineered 5-repeat microdystrophin protein that contains, from N- to C-terminus, the N-terminal actin binding domain, Hinge region 1 (H1), spectrin-like repeats R1, R16, R17, R23, and R24, Hinge region 4 (H4), and the C-terminal dystroglycan binding domain of the human full-length dystrophin protein. The protein sequence of this 5-repeat microdystrophin and the related dystrophin minigene are described in U.S. Pat. No. 10,479,821 & WO2016/115543 (incorporated herein by reference).

In certain embodiments, the dystrophin minigene encoding a functional dystrophin protein different from microD5 with respect to, for example, the specific spectrin-like repeats, and/or the number of spectrin-like repeats (e.g., comprising a minimum of 4, 5, or 6 spectrin-like repeats of the human dystrophin, preferably including 1, 2, or 3 most N- and/or most C-terminal repeats). One or more spectrin-like repeats of the human dystrophin may also be substituted by spectrin-like repeats from utrophin or spectrin. In certain embodiments, the dystrophin minigene is smaller than the 5 kb packaging limit of AAV viral vectors, preferably no more than 4.9 kb, 4.8 kb, 4.6 kb, 4.5 kb, 4.4 kb, 4.3 kb, 4.2 kb, 4.1 kb, or 4 kb.

In certain embodiments, the dystrophin minigene encodes a micro-dystrophin protein that is, e.g., at least 65%, at least 70%, at least 75%, at least 80%, 81%, 82%, 83%, 84%, 85%, 86%, 87%, 88%, or 89%, more typically at least 90%, 91%, 92%, 93%, or 94% and even more typically at least 95%, 96%, 97%, 98% or 99% sequence identity to microD5, wherein the protein retains micro-dystrophin activity.

In certain embodiments, the micro-dystrophin is encoded by a nucleotide sequence that has at least 65%, at least 70%, at least 75%, at least 80%, 81%, 82%, 83%, 84%, 85%, 86%, 87%, 88%, or 89%, more typically at least 90%, 91%, 92%, 93%, or 94% and even more typically at least 95%, 96%, 97%, 98% or 99% sequence identity to a polynucleotide sequence encoding the microD micro-dystrophin. The polynucleotide is optionally codon optimized for expression in a mammal, such as in a human.

In certain embodiments, the nucleotide sequence hybridizes under stringent conditions to the nucleic acid sequence encoding the microD5 micro-dystrophin, or compliments thereof, and encodes a functional micro-dystrophin protein.

The term “stringent” is used to refer to conditions that are commonly understood in the art as stringent. Hybridization stringency is principally determined by temperature, ionic strength, and the concentration of denaturing agents such as formamide. Examples of stringent conditions for hybridization and washing are 0.015 M sodium chloride, 0.0015 M sodium citrate at 65-68° C. or 0.015 M sodium chloride, 0.0015 M sodium citrate, and 50% formamide at 42° C. See Sambrook et al., Molecular Cloning: A Laboratory Manual, 2nd Ed., Cold Spring Harbor Laboratory, (Cold Spring Harbor, N.Y. 1989).

More stringent conditions (such as higher temperature, lower ionic strength, higher formamide, or other denaturing agent) may also be used, however, the rate of hybridization will be affected. In instances wherein hybridization of deoxyoligonucleotides is concerned, additional exemplary stringent hybridization conditions include washing in 6×SSC 0.05% sodium pyrophosphate at 37° C. (for 14-base oligoes), 48° C. (for 17-base oligos), 55° C. (for 20-base oligos), and 60° C. (for 23-base oligos).

Other agents may be included in the hybridization and washing buffers for the purpose of reducing non-specific and/or background hybridization. Examples are 0.1% bovine serum albumin, 0.1% polyvinyl-pyrrolidone, 0.1% sodium pyrophosphate, 0.1% sodium dodecylsulfate, NaDodS04, (SDS), ficoll, Denhardt's solution, sonicated salmon sperm DNA (or other non-complementary DNA), and dextran sulfate, although other suitable agents can also be used. The concentration and types of these additives can be changed without substantially affecting the stringency of the hybridization conditions. Hybridization experiments are usually carried out at pH 6.8-7.4, however, at typical ionic strength conditions, the rate of hybridization is nearly independent of pH. See Anderson et al., Nucleic Acid Hybridisation: A Practical Approach, Ch. 4, IRL Press Limited (Oxford, England). Hybridization conditions can be adjusted by one skilled in the art in order to accommodate these variables and allow DNAs of different sequence relatedness to form hybrids.

Additional dystrophin minigene sequences can be found in, for example, US2017/0368198 (incorporated herein by reference), and SEQ ID NO: 7 of WO 2017/181015 (incorporated herein by reference).

In certain embodiments, the nucleotide sequence encoding any dystrophin minigene such as microD5 can be any one based on the disclosed protein sequence. Preferably, the nucleotide sequence is codon optimized for expression in human.

The micro-dystrophin protein provides stability to the muscle membrane during muscle contraction, e.g., micro-dystrophin acts as a shock absorber during muscle contraction.

In certain embodiments, at least one of the one or more coding sequences target one of the secondary cascade genes in DMD.

For example, in certain embodiments, at least one of the one or more coding sequences encodes a microRNA, such as miR-1, miR-133a, miR-29 particularly miR29c, miR-30c, and/or miR-206. For example, miR-29c directly reduce the three primary components of connective tissue (e.g., collagen 1, collagen 3 and fibronectin) to reduce fibrosis. Optionally, in certain embodiments, said microRNA, such as miR-1, miR-133a, miR-29 particularly miR29c, miR-30c, and/or miR-206, has a modified flanking backbone sequence that enhances the processing of the guide strand designed for a target sequence. In certain embodiments, the modified flanking backbone sequence can be from or based on miR-30, -101, -155, or -451.

“Fibrosis” as used herein refers to the excessive or unregulated deposition of extracellular matrix (ECM) components and abnormal repair processes in tissues upon injury including skeletal muscle, cardiac muscle, liver, lung, kidney, and pancreas. The ECM components that are deposited include fibronectin and collagen, e.g., collagen 1, collagen 2 or collagen 3.

As used herein, “miR-29” refers to one of miR-29a, -29b, or -29c. In certain embodiments, miR-29 refers to miR-29c.

While not wishing to be bound by any particular theory, it is believed that the expressed miR29 (such as miR-29a, miR-29b, or miR-29c) binds to the 3′ UTR of the collagen and fibronectin genes to down-regulate expression of these target genes.

In another embodiment, at least one of the one or more coding sequences encodes an RNAi sequence, such as an shRNA against sarcolipin (shSLN). The one or more coding sequences may encode identical or different shRNAs against sarcolipin (shSLN). In certain embodiments, the shRNA reduces sarcolipin mRNA and/or sarcolipin protein expression by at least about 50%.

As used herein, “sarcolipin (SLN),” “sarcolipin protein,” “SLN protein,” “sarcolipin polypeptide” and “SLN polypeptide” are used interchangeably to include an expression product of a SLN gene, such as the native human SLN protein having the amino acid sequence of (MGINTRELFLNFTIVLITVILMWLLVRSYGY) (SEQ ID NO: 5), accession number NP_003054.1. The term preferably refers to the human SLN. The term may also be used to refer to a variant SLN protein that differs from SEQ ID NO: 5 by 1 amino acid, 2 amino acids, 3 amino acids, 4 amino acids, 5 amino acids, 6 amino acids, 7 amino acids, or 8 amino acids, optionally the differences are within residues 2-5, 10, 14, 17, 20, and 30, preferably 2-5 and 30. The term may also be used to refer to a variant SLN protein that are identical to SEQ ID NO: 5 at residues 6-29, or differ in residues 6-29 by up to 1, 2, or 3 conservative substitutions such as L→I and/or I→V. Optionally, the variant SLN has a G30Q substitution. The variants displays a functional activity of a native SLN protein, which may include: phosphorylation, dephosphorylation, nitrosylation and/or ubiquitination of SLN, or binding to a SERCA and/or reduce the rate of calcium import by SERCA into the sarcoendoplasmic reticulum through, for example, uncoupling of Ca²⁺ transport from ATP hydrolysis, or its role in energy metabolism and regulation of weight gain.

As used herein, “SLN gene,” “SLN polynucleotide,” and “SLN nucleic acid” are used interchangeably to include a native human SLN-encoding nucleic acid sequence, e.g., the native human SLN gene (RefSeq Accession: NM_003063.2), a nucleic acid having sequences from which a SLN cDNA can be transcribed; and/or allelic variants and homologs of the foregoing, such as a polynucleotide encoding any of the variant SLN described herein. The terms encompass double-stranded DNA, single-stranded DNA, and RNA.

In another embodiment, the one or more additional coding sequences of the subject vector may be targeting any other genes associated with one of the secondary cascade events resulting from the loss of dystrophin gene, such as inflammatory gene, an activator of NF-κB signaling pathway (e.g., TNF-α, IL-1, IL-1β, IL-6, Receptor activator of NF-κB (RANK), and Toll-like receptors (TLRs)), NF-κB, a downstream inflammatory cytokine induced by NF-κB, a histone deacetylase (e.g., HDAC2), TGF-β, connective tissue growth factor (CTGF), ollagens, elastin, a structural component of the extracellular matrix, Glucose-6-phosphate dehydrogenase (G6PD), myostatin, phosphodiesterase-5 (PED-5) or ACE, VEGF decoy-receptor type 1 (VEGFR-1 or Flt-1), and hematopoietic prostaglandin D synthase (HPGDS). The one or more additional coding sequences can be an RNAi sequence (siRNA, shRNA, miRNA), an antisense sequence, and/or a microRNA that antagonizes the function of the above target genes.

The design of the subject recombinant vectors can simultaneously target one or more (e.g., 1, 2, 3, 4, 5) such secondary cascade genes or pathways, such as SLN, microRNA, etc.

For example, in certain embodiments, one of the additional coding sequence of the subject vector may be an RNAi sequence (siRNA, shRNA, miRNA) or an antisense sequence designed to down-regulate SLN expression, hence at least partially alleviate the secondary defect of abnormal elevation of intracellular Ca²⁺ in dystrophy muscle by increasing the reuptake of calcium by SERCA.

In certain alternative embodiments, instead of or in addition to targeting one of the secondary cascade genes, at least one of the one or more coding sequences may be an exon-skipping antisense sequence that induces skipping of an exon of a defective endogenous dystrophin, such as any one of exons 45-55 of dystrophin, or exon 44, 45, 51, and/or 53 of dystrophin, thus further enhancing the therapeutic effect of the dystrophin minigene (e.g., microD5).

As used herein, an “exon skipping” or “splice-switching” antisense oligonucleotide (AON) is a type of antisense sequence that is RNase-H-resistant, and acts to modulate pre-mRNA splicing and correct splicing defects in the pre-mRNA. In antisense-mediated exon skipping therapy, AONs are usually used to block specific splicing signals and induce specific skipping of certain exons. This leads to the correction of the reading frame of a mutated transcript, such that it can be translated into an internally deleted but partially functional protein.

In a specific aspect, the invention provides a recombinant AAV (rAAV) vector encoding both a dystrophin minigene coding sequence (such as microD5/SGT-001), and one or more additional sequences for targeting one or more additional target genes involved in a secondary cascade resulting from loss of dystrophin function. Such construct comprises both a dystrophin minigene, and one or more additional coding sequences inserted between the first control element or promoter of the dystrophin minigene and the nearest viral terminal sequence (e.g., ITR in AAV) and operably linked to a second control element, and (2) optionally further inserted into heterologous intron 5′ to the dystrophin minigene or 3′-UTR region of the dystrophin minigene.

Specifically, in one aspect, the invention provides a recombinant AAV (rAAV) vector comprising: a) a dystrophin minigene encoding a functional micro-dystrophin protein, wherein said dystrophin minigene comprises a 3′-UTR coding region, and is immediately 3′ to a heterologous intron sequence that enhances expression of the dystrophin minigene; b) a muscle-specific control element operably linked to and drives the expression of the dystrophin minigene; and, c) one or more (e.g., 1, 2, 3, 4, or 5) coding sequence(s) inserted between the muscle-specific control element and the nearest AAV ITR sequence and operably linked to a second control element, and (2) optionally further inserted into the intron sequence or in the 3′-UTR coding region; wherein said one or more coding sequence(s) independently encode(s): an RNAi sequence (siRNA, shRNA, miRNA), an antisense sequence, a microRNA (miRNA), and/or a miRNA inhibitor.

For example, the rAAV vector may comprise a polynucleotide sequence expressing miR-29 (e.g., miR-29c), such as a nucleotide sequence comprising the miR-29c target guide strand (ACCGATTTCAAATGGTGCTAGA, SEQ ID NO: 3 of WO2017/181015 or, incorporated herein by reference), the miR-29c guide strand (TCTAGCACCATTTGAAATCGGTTA, SEQ ID NO: 4 of WO2017/181015, incorporated herein by reference) and the natural miR-30 back bone and stem loop (GTGAAGCCACAGATG, SEQ ID NO: 5 of WO2017/181015, incorporated herein by reference).

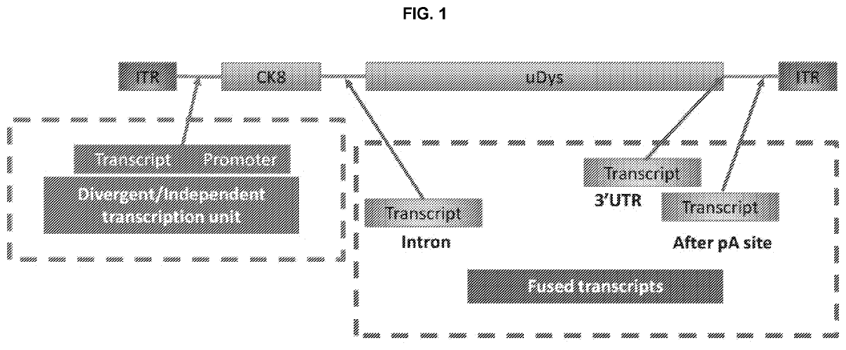

An exemplary polynucleotide sequence comprising the miR-29c cDNA in a miR-30 backbone is set out as SEQ ID NO: 2 and FIG. 1 of WO2017/181015 (incorporated herein by reference).

In certain embodiments, the microRNA-29 coding sequence encodes miR-29c.

In certain embodiments, miR-29c optionally has a modified flanking backbone sequence that enhances the processing of the guide strand of miR-29c designed for a target sequence. For example, the modified flanking backbone sequence can be from or based on that of miR-30 (miR-30E), -101, -155, or -451.

In certain embodiments, the microRNA is miR-1, miR-133a, miR-30c, and/or miR-206.

In certain embodiments, expression of said microRNA in a host cell is up-regulated by at least about 1.5-15 fold (e.g., about 2-10 fold, about 1.4-2.8 fold, about 2-5 fold, about 5-10 fold, about 2, 3, 4, 5, 6, 7, 8, 9, 10, 11, 12, 13, 14, or about 15 fold) compared to endogenous expression of said microRNA in said host cell.

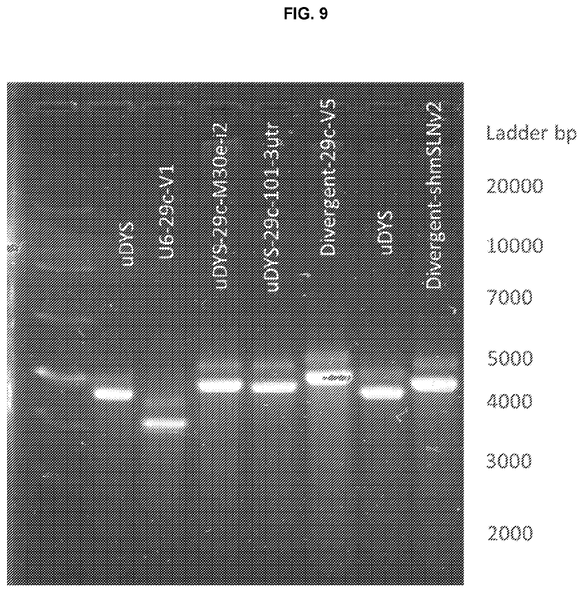

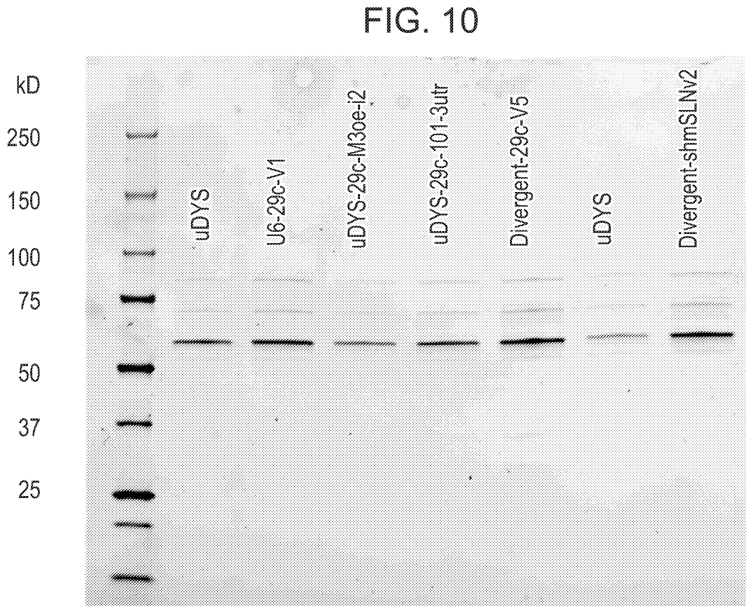

In certain embodiments, the vector of the invention encodes an antisense sequence, or an RNAi sequence (siRNA, shRNA, miRNA etc.), that antagonizes the function of sarcolipin (SLN). In certain embodiments, the vector of the invention encodes an shRNA that antagonizes the function of sarcolipin (shSLN). Exemplary shSLN sequences include those disclosed in FIGS. 9 and 10 of PCT/US2019/065718, filed on Dec. 11, 2019 (e.g., the underlined sequences in FIG. 9, and the highlighted sequences in FIG. 10). Additional exemplary shSLN sequences include SEQ ID NOs: 7-11 disclosed in WO2018/136880 (incorporated herein by reference).

The invention is also partly directed to gene therapy vectors, e.g., lentiviral or AAV expressing the one or more coding sequence(s), such as the dystrophin minigene, as well as methods of using the same for treating disease, e.g., delivering the same to the muscle to reduce and/or prevent a secondary cascade symptom while restoring dystrophin function.

In one embodiment, the muscular dystrophy is a congenital muscular dystrophy (CMD) associated with a known genetic defect, such as the fukutin gene or the FKRP (fukutin related protein) gene. Thus in certain embodiments, the congenital muscular dystrophy is Fukuyama congenital muscular dystrophy (FCMD).

Congenital Muscular Dystrophy (CMD) is a group of muscular dystrophies that become apparent at or near birth. In certain embodiments, the methods and rAAV of the invention can be used to treat CMD, especially CMD with known genetic defect in genes such as titin (CMD with cardiomyopathy); SEPN1 (CMD with desmin inclusions, or CMD with (early) spinal rigidity); integrin-alpha 7 (CMD with integrin alpha 7 mutations); integrin-alpha 9 (CMD with joint hyperlaxity); plectin (CMD with familial junctional epidermolysis bullosa); fukutin (Fukuyama CMD or MDDGA4); fukutin-related protein (FKRP) (CMD with muscle hypertrophy or MDC1C); LARGE (MDC1D); DOK7 (CMD with myasthenic syndrome); lamin A/C (CMD with spinal rigidity and lamin A/C abnormality); SBP2 (CMD with spinal rigidity and selenoprotein deficiency); choline kinase beta (CMD with structural abnormalities of mitochondria); laminin alpha 2 (Merosin-deficient CMD or MDC1A); POMGnT1 (Santavuori muscle-eye-brain disease); COLGA1, COL6A2, or COL6A3 (Ullrich CMD); B3GNT1 (Walker-Warburg syndrome: MDDGA type); POMT1 (Walker-Warburg syndrome: MDDGA1 type); POMT2 (Walker-Warburg syndrome: MDDGA2 type); ISPD (MDDGA3, MDDGA4, MDDGB5, MDDGA6, and MDDGA7); GTDC2 (MDDGA8); TMEM5 (MDDGA10); B3GALNT2 (MDDGA11); or SGK196 (MDDGA12).

Thus the lentiviral or rAAV vector of the invention may comprise a polynucleotide encoding any of the wild-type genes defective in the CMD (such as the ones listed herein above), or a functional equivalent thereof, to treat the CMD in a subject in need thereof. The one or more additional coding sequences may encode an RNAi sequence (siRNA, shRNA, miRNA), an antisense sequence, or a microRNA (miRNA) that eliminates or modifies the mutant CMD gene, or a secondary cascade gene up-regulated due to the loss of the wild-type gene function.

For example, Fukuyama congenital muscular dystrophy (FCMD) is due to a mutant FKTN gene, and the one or more additional coding sequences may encode an exon-skipping antisense oligonucleotide to restore correct exon 10 splicing in the defective FKTN gene in the patient.

In another example, the congenital muscular dystrophy is Merosin-deficient congenital muscular dystrophy type 1A (MDC1A) caused by mutations in the 65-exon LAMA2 gene.

Thus the lentiviral or rAAV vector of the invention may comprise a polynucleotide encoding a functional LAMA2 protein. The one or more additional coding sequences may encode an exon-skipping antisense sequence leading to the restored expression of the C-terminal G-domain (exons 45-64), particularly G4 and G5 of LAMA2 that are most important for mediating interaction with α-dystroglycan. For example, exon 4 of the mutant LAMA2 gene may be skipped to treat MDC1A.

In one embodiment, the muscular dystrophy is myotonic dystrophy (DM), such as DM1 or DM2.

Thus the lentiviral or rAAV vector of the invention may comprise a polynucleotide encoding a functional Dystrophia Myotonica Protein Kinase (DMPK) protein defective in DM1, or a functional CCHC-type zinc finger, nucleic acid binding protein gene (CNBP) protein in DM2. The one or more additional coding sequences may encode an RNAi sequence (siRNA, shRNA, miRNA), an antisense sequence, or a microRNA (miRNA) that can be used to target expanded repeats of mutant transcripts in the DMPK gene or the CNBP gene for RNase-mediated degradation. The one or more additional coding sequences may also encode an exon-skipping antisense sequence leading to the skipping of exon 7A in CLCN1 gene in a DM1 patient.

In one embodiment, the muscular dystrophy is Dysferlinopathy caused by mutations in the dysferlin (DYSF) gene, including limb-girdle muscular dystrophy type 2B (LGMD2B) and Miyoshi myopathy (MM).

Thus the lentiviral or rAAV vector of the invention may comprise a polynucleotide encoding a functional DYSF protein defective in LGMD2B or MM. The one or more additional coding sequences may encode an exon-skipping antisense sequence leading to the skipping of exon 32 in a defective DYSF gene in a dysferlinopathy patient.

In one embodiment, the muscular dystrophy is limb-girdle muscular dystrophy (LGMD) caused by mutations in any of the four sarcoglycans genes, namely α (LGMD2D), β (LGMD2E), γ (LGMD2C) and δ (LGMD2F) gene, particularly the γ sarcoglycan (LGMD2C) encoded by the SGCG gene.

Thus the lentiviral or rAAV vector of the invention may comprise a polynucleotide encoding a functional sarcoglycan protein defective in a LGMD, such as the SGCG gene defective in LGMD2C. The one or more additional coding sequences may encode an exon-skipping antisense sequence leading to the skipping of exons 4-7 in a defective LGMD2C gene, such as one with the Δ-521T SGCG mutation.

In one embodiment, the muscular dystrophy is Facioscapulohumeral muscular dystrophy (FSHD) caused by mutations in the DUX4 gene.

Thus the one or more additional coding sequences may encode an RNAi sequence (siRNA, shRNA, miRNA), an antisense sequence, or a microRNA (miRNA) that reduces the expression of DUX4 or a downstream target such as PITX1.

In certain embodiments, the one or more additional coding sequences encode an exon skipping antisense sequence that targets 3′-UTR of DUX4 to reduce its expression. This is because the DUX4 coding sequence is entirely located in the gene first exon, and exon skipping that targets elements in the mRNA 3′ UTR can either disrupt the permissive polyadenylation or interfere with intron 1 or 2 splicing, hence destroying a functional DUX4 mRNA.

Facioscapulohumeral muscular dystrophy (FSHD) is an inherited autosomal dominant disorder characterized clinically by progressive muscle degeneration. It is the third most common muscular dystrophy after Duchenne muscular dystrophy (DMD) and myotonic dystrophy. FSHD is genetically characterized by a pathogenic contraction of a subset of macrosatellite repeats on chromosome 4, leading to aberrant expression of the double homeobox protein 4 (DUX4) gene.

There are two types of FSHD: FSHD1 and FSHD2. FSHD1 is the most common form that occurs in over 95% of all FSHD patients. Genetic analysis links FSHD 1 to the genetic contraction of macrosatellite D4Z4 repeat array on chromosome 4. FSHD2, on the other hand, has a normal number of D4Z4 repeats but instead involves a heterozygous mutation in the SMCHD1 gene on chromosome 18p, a chromatin modifier. Patients with FSHD1 and FSHD2 share similar clinical presentations.

Current drug therapy does not cure FSHD, but focus on the management of FSHD symptoms, including myostatin inhibitor luspatercept and anti-inflammatory biologics (ATYR1940). The basis for anti-inflammatory biologics is to suppress inflammation commonly seen in muscle pathology of FSHD patients in order to slow phenotype progression. Thus the subject one or more coding sequences may encode an RNAi reagent or antisense RNA against myostatin or an inflammatory pathway gene. Meanwhile, the RNAi reagent such as small interfering RNA (siRNA) and small hairpin RNA (shRNA), or microRNA (miRNA), or antisense oligonucleotides, can be used to knockdown expression of the myopathic DUX4 gene and its downstream molecules including paired-like homeodomain transcription factor 1 (PITX1). Indeed, in vitro studies have demonstrated success in the suppression of DUX4 mRNA expression by administering antisense oligoes into primary skeletal muscle cells of FSHD patients, and by using miRNA against DUX4 delivered to a DUX4 mouse model using AAV vector. In addition, success in the suppression of PITX1 expression has already been demonstrated systemically in vivo.

In certain embodiments, the one or more additional coding sequences can encode the same sequence (e.g., siRNA, shRNA, miRNA, or antisense), and thus the copy number of the additional coding sequence may be regulated or fine tuned for dosing consideration.

In certain embodiments, the one or more additional coding sequences can encode different sequences, either targeting different targets, or targeting the same target. For example, in certain embodiments, one additional coding sequence is an antisense against a target, while another additional coding sequence is an shRNA against the same target. Alternatively, two additional coding sequences are both shRNAs but they target different regions of the same target.

In certain embodiments, expression of the functional protein, such as the dystrophin minigene product, is not negatively affected by the insertion of the one or more coding sequence(s).

By early 1990s, it has been found that many intronless transgenes, while express perfectly in tissue culture cells in vitro, fail to express the same transgene in vivo (e.g., in transgenic mice harboring the transgene), while inserting certain heterologous intron sequences between the promoter and the (intronless) coding sequence of the transgene greatly enhances transgene expression in vivo.

In particular, Palmiter et al. (Proc. Natl. Acad. Sci. U.S.A. 88:478-482, 1991, incorporated herein by reference) showed that several heterologous introns inserted between the metallothionein promoter and the growth hormone transgene improves transgene expression, and provided addition of certain heterologous introns as a general strategy for improving transgene expression. These include heterologous introns selected from: the natural first intron of rGH, intron A of the rat insulin II (rIns-II) gene, intron B of the hβG gene, and the SV40 small t intron.

A similar finding was confirmed by Choi et al. (Mol. Cell. Biol. 11(6):3070-3074, 1991, incorporated herein by reference), who reported that in transgenic mice carrying the human histone H4 promoter linked to the bacterial gene for chloramphenicol acetyltransferase (CAT), the presence of a 230-bp heterologous hybrid intron in the transcription unit greatly enhanced CAT activity (by 5- to 300-fold, compared to an analogous transgene precisely deleted for the intervening sequences). This hybrid intron, consisting of an adenovirus splice donor and an immunoglobulin G splice acceptor, stimulated expression in a broad range of tissues in the animal. Since the hybrid intron stimulated the expression of tissue plasminogen activator and factor VIII in tissue culture, Choi concluded that the enhancement seen in mice is unlikely to be specific to CAT and instead is generally applicable to the expression of any cDNAs in transgenic mice.

Thus in certain embodiment, the heterologous intron in the subject lentiviral or rAAV vector is selected from the group consisting of: the natural first intron of rGH, intron A of the rat insulin II (rIns-II) gene, intron B of the hβG gene, the SV40 small t intron, and the hybrid intron of Choi.

In certain embodiments, the heterologous intron sequence is SEQ ID NO: 1:

GTATCAAGGTTACAAGACAGGTTTAAGGAGACCAATAGAAACTGGGCTTG
TCGAGACAGAGAAGACTCTTGCGTTTCTGATAGGCACCTATTGGTCTTAC
TGACATCCACTTTGCCTTTCTCTCCACAG.

In certain embodiments, in addition to being inserted into the divergent cassette (e.g., between the GOI promoter and the nearest AAV ITR), the one or more additional coding sequences are all inserted into the heterologous intron sequence (SEQ ID NO: 1), or all inserted into the 3′-UTR region, or are inserted into both regions. For example, the microRNA-29c coding sequence can be inserted into the intron coding sequence as in SEQ ID NO: 2

GTATCAAGGTTACAAGACAGGTTTAAGGAGACCAATAGAAACTGGGCTTG
TCGAGACAGATCTCTTACACAGGCTGACCGATTTCTCCTGGTGTTCAGAG
TCTGTTTTTGTCTAGCACCATTTGAAATCGGTTATGATGTAGGGGGAAGA
AGACTCTTGCGTTTCTGATAGGCACCTATTGGTCTTACTGACATCCACTT
TGCCTTTCTCTCCACAG.

The miR-29c sequence in SEQ ID NO: 2 is

(SEQ ID NO: 3)
ATCTCTTACACAGGCTGACCGATTTCTCCTGGTGTTCAGAGTCTGTTTTT
GTCTAGCACCATTTGAAATCGGTTATGATGTAGGGGGA.

In certain embodiments, the lentiviral or rAAV further comprises two lentiviral or AAV LTR/ITR sequences flanking the polynucleotide (such as the dystrophin minigene) and the additional coding sequence(s).

In certain embodiments, the GOI encoded by the lentiviral or rAAV vectors of the invention may be operably linked to a muscle-specific control element. For example, the muscle-specific control element can be: human skeletal actin gene element, cardiac actin gene element, myocyte-specific enhancer binding factor MEF, muscle creatine kinase (MCK), tMCK (truncated MCK), myosin heavy chain (MHC), C5-12 (synthetic promoter), murine creatine kinase enhancer element, skeletal fast-twitch troponin C gene element, slow-twitch cardiac troponin C gene element, slow-twitch troponin I gene element, hypozia-inducible nuclear factors, steroid-inducible element, or glucocorticoid response element (GRE).

In certain embodiments, muscle-specific control element is 5′ to the heterologous intron sequence, which is 5′ to the dystrophin minigene, which comprises a 3′-UTR region including a translation stop codon (such as TAG), a polyA adenylation signal (such as AATAAA), and an mRNA cleavage site (such as CA).

In certain embodiments, the muscle-specific control element comprises the nucleotide sequence of SEQ ID NO: 10 or SEQ ID NO: 11 of WO2017/181015.

| SEQ ID NO: 10 of WO2017/181015: | |
|---|---|
| CAGCCACTAT GGGTCTAGGC TGCCCATGTA AGGAGGCAAG GCCTGGGGAC ACCCGAGATG | 60 |
| CCTGGTTATA ATTAACCCAG ACATGTGGCT GCTCCCCCCC CCCAACACCT GCTGCCTGAG | 120 |
| CCTCACCCCC ACCCCGGTGC CTGGGTCTTA GGCTCTGTAC ACCATGGAGG AGAAGCTCGC | 180 |
| TCTAAAAATA ACCCTGTCCC TGGTGG | 206 |
| SEQ ID NO: 11 of WO2017/181015: | |
| GCTGTGGGGG ACTGAGGGCA GGCTGTAACA GGCTTGGGGG CCAGGGCTTA TACGTGCCTG | 60 |
| GGACTCCCAA AGTATTACTG TTCCATGTTC CCGGCGAAGG GCCAGCTGTC CCCCGCCAGC | 120 |
| TAGACTCAGC ACTTAGTTTA GGAACCAGTG AGCAAGTCAG CCCTTGGGGC AGCCCATACA | 180 |
| AGGCCATGGG GCTGGGCAAG CTGCACGCCT GGGTCCGGGG TGGGCACGGT GCCCGGGCAA | 240 |
| CGAGCTGAAA GCTCATCTGC TCTCAGGGGC CCCTCCCTGG GGACAGCCCC TCCTGGCTAG | 300 |
| TCACACCCTG TAGGCTCCTC TATATAACCC AGGGGCACAG GGGCTGCCCC CGGGTCAC | 358 |

In certain embodiments, the rAAV vectors of the invention can be operably linked to the muscle-specific control element comprising the MCK enhancer nucleotide sequence (see SEQ ID NO: 10 of WO2017/181015, incorporated herein by reference) and/or the MCK promoter sequence (see SEQ ID NO: 11 of WO2017/181015, incorporated herein by reference).

In certain embodiments, the rAAV further comprises a promoter operably linked to and is capable of driving the transcription of the dystrophin minigene and the additional coding sequence.

An exemplary promoter is the CMV promoter.

In certain embodiments, the rAAV further comprises a poly-A adenylation sequence for inserting a polyA sequence into a transcribed mRNA.

In certain embodiments, the rAAV vectors of the invention are of the serotype AAV1, AAV2, AAV4, AAV5, AAV6, AAV7, AAVrh.74, AAV8, AAV9, AAV10, AAV11, AAV12 or AAV13.

Another aspect of the invention provides a method of producing a viral vector, e.g., rAAV vector of the invention, comprising culturing a cell that has been transfected with any viral vector, e.g., rAAV vector of the invention and recovering the virus, e.g., rAAV particles from the supernatant of the transfected cells.

Another aspect of the invention provides viral particles comprising any of the viral vector, e.g., recombinant AAV vectors of the invention.

Another aspect of the invention provides methods of producing a functional protein either defective in a muscular dystrophy, or effective to treat the muscular dystrophy (such as a micro-dystrophin protein), and one or more additional coding sequence(s), comprising infecting a host cell with a subject recombinant AAV vector co-expressing the functional protein (e.g., micro-dystrophin) of the invention and the coding sequence product (e.g., RNAi, siRNA, shRNA, miRNA, antisense, microRNA or inhibitor thereof) in the host cell.

Another aspect of the invention provides methods of treating a muscular dystrophy (such as DMD or BMD) or dystrophinopathy in a subject in need thereof, the method comprising administering to the subject a therapeutically effective amount of a viral vector, e.g., recombinant AAV vector of the invention, or a composition of the invention.

The invention contemplates administering any of the viral vector, e.g., AAV vectors of the invention to patients diagnosed with dystrophinopathy or muscular dystrophy, such as DMD or BMD or any other MD, particularly defective dystrophin-associated muscular dystrophy, preferably before one or more secondary cascade symptoms such as fibrosis is observed in the subject, or before the muscle force has been reduced in the subject, or before the muscle mass has been reduced in the subject.

The invention also contemplates administering any of the viral vector, e.g., rAAV of the invention to a subject suffering from dystrophinopathy or muscular dystrophy, such as DMD or BMD or any other MD, particularly dystrophin-associated muscular dystrophy, who already has developed one or more secondary cascade symptoms such as fibrosis, in order to prevent or slow down further disease progression in these subjects.

Another aspect of the invention provides recombinant viral vector, e.g., AAV vectors comprising a nucleotide sequence encoding a functional protein either defective in a muscular dystrophy, or effective to treat the muscular dystrophy (e.g., a micro-dystrophin protein) and the one or more additional coding sequences.

In certain embodiments, the invention provides for a rAAV comprising a nucleotide sequence having at least 85%, 90%, 95%, 97%, or 99% identity to the nucleotide sequence that encodes a functional micro-dystrophin protein such as microD5.

The viral vector, e.g., rAAV vector may comprise a muscle-specific promoter, such as the MCK promoter, a heterologous intron sequence effective to enhance the expression of the dystrophin gene, the coding sequence for the micro-dystrophin gene, polyA adenylation signal sequence, the ITR/LTR repeats flanking these sequences. The viral vector, e.g., rAAV vector may optionally further comprises ampicillin resistance and plasmid backbone sequences or pBR322 origin or replication for amplification in a bacteria host.

In one aspect, the recombinant AAV vectors of the invention are AAV1, AAV2, AAV4, AAV5, AAV6, AAV7, AAVrh.74, AAV8, AAV9, AAV10, AAV 11, AAV 12 or AAV 13.

In any of the methods of the invention, the rAAV vector can be administered by intramuscular injection or intravenous injection.

In any of the methods of the invention, the viral vector, e.g., rAAV vector or composition is administered systemically. For examples, the viral vector, e.g., rAAV vector or composition is parentally administration by injection, infusion or implantation.

Another aspect of the invention provides a composition, such as a pharmaceutical composition, comprising any of the viral vector, e.g., rAAV vectors of the invention.

In certain embodiments, the composition is a pharmaceutical composition, which may further comprise a therapeutically compatible carrier or excipient.

In another embodiment, the invention provides for composition comprising any of the viral vector, e.g., rAAV vectors co-expressing the subject functional protein (e.g., micro-dystrophin) and said one or more additional coding sequences for treating a subject suffering from dystrophinopathy or a muscular dystrophy, such as DMD or Becker Muscular dystrophy.

The compositions (e.g., pharmaceutical compositions) of the invention can be formulated for intramuscular injection or intravenous injection. The composition of the invention can also be formulated for systemic administration, such as parentally administration by injection, infusion or implantation. In addition, any of the compositions are formulated for administration to a subject suffering from dystrophinopathy or a muscular dystrophy, such as DMD, Becker muscular dystrophy or any other dystrophin associated muscular dystrophy.

In a further embodiment, the invention provides for use of any of the viral vector, e.g., rAAV vectors of the invention co-expressing a subject functional protein (e.g., a micro-dystrophin) and said one or more additional coding sequences for preparation of a medicament for reducing the subject suffering from dystrophinopathy or muscular dystrophy, such as DMD, Becker muscular dystrophy or any other dystrophin associated muscular dystrophy.

The invention contemplates use of the any of the viral vector, e.g., AAV vectors of the invention for the preparation of a medicament for administration to a patient diagnosed with DMD before one or more secondary cascade symptoms such as fibrosis is observed in the subject.

The invention also contemplates use of any of the viral vector, e.g., AAV vectors of the invention for the preparation of a medicament for administering any of the viral vector, e.g., rAAV of the invention to a subject suffering from muscular dystrophy who already has developed a secondary cascade symptom such as fibrosis, in order to prevent or delay disease progression in these subjects.

The invention also provides for use of the viral vector, e.g., rAAV vectors of the invention co-expressing a subject functional protein such as a micro-dystrophin, and said one or more additional coding sequences for the preparation of a medicament for treatment of a muscular dystrophy, such as DMD/BMD.

In any of the uses of the invention, the medicament can be formulated for intramuscular injection. In addition, any of the medicaments may be prepared for administration to a subject suffering from muscular dystrophy such as DMD or any other dystrophin associated muscular dystrophy.

The present invention also provides for gene therapy vectors, e.g., rAAV vectors that co-express a subject functional protein (e.g., a micro-dystrophin) and said one or more additional coding sequences in a muscular dystrophy patient.

It should be understand that any one embodiment of the invention described herein can be combined with any one or more additional embodiments of the invention, including those embodiments described only in the examples or only described in one of the sections above or below, or one aspect of the invention.

### AAV

As used herein, the term “AAV” is a standard abbreviation for adeno-associated virus. Adeno-associated virus is a single-stranded DNA parvovirus that grows only in cells in which certain functions are provided by a co-infecting helper virus. There are at least thirteen serotypes of AAV that have been characterized. General information and reviews of AAV can be found in, for example, Carter, 1989, Handbook of Parvoviruses, Vol. 1, pp. 169-228, and Berns, 1990, Virology, pp. 1743-1764, Raven Press, (New York) (incorporated herein by reference). However, it is fully expected that these same principles will be applicable to additional AAV serotypes since it is well known that the various serotypes are quite closely related, both structurally and functionally, even at the genetic level. See, for example, Blacklowe, 1988, pp. 165-174 of Parvoviruses and Human Disease, J. R. Pattison, ed.; and Rose, Comprehensive Virology 3: 1-61 (1974). For example, all AAV serotypes apparently exhibit very similar replication properties mediated by homologous rep genes; and all bear three related capsid proteins such as those expressed in AAV2. The degree of relatedness is further suggested by heteroduplex analysis which reveals extensive cross-hybridization between serotypes along the length of the genome; and the presence of analogous self-annealing segments at the termini that correspond to “inverted terminal repeat sequences” (ITRs). The similar infectivity patterns also suggest that the replication functions in each serotype are under similar regulatory control.

An “AAV vector” as used herein refers to a vector comprising one or more polynucleotides of interest (or transgenes) that are flanked by AAV terminal repeat sequences (ITRs). Such AAV vectors can be replicated and packaged into infectious viral particles when present in a host cell that has been transfected with a vector encoding and expressing rep and cap gene products.

An “AAV virion” or “AAV viral particle” or “AAV vector particle” refers to a viral particle composed of at least one AAV capsid protein and an encapsidated polynucleotide AAV vector. If the particle comprises a heterologous polynucleotide (i.e., a polynucleotide other than a wild-type AAV genome such as a transgene to be delivered to a mammalian cell), it is typically referred to as an “AAV vector particle” or simply an “AAV vector.” Thus, production of AAV vector particle necessarily includes production of AAV vector, as such a vector is contained within an AAV vector particle.

Recombinant AAV genomes of the invention comprise nucleic acid molecule of the invention and one or more AAV ITRs flanking a nucleic acid molecule.

There are multiple serotypes of AAV, and the nucleotide sequences of the genomes of the AAV serotypes are known. For example, the nucleotide sequence of the AAV serotype 2 (AAV2) genome is presented in Srivastava et al., J Virol 45:555-564 (1983) as corrected by Ruffing et al., J Gen Virol 75:3385-3392 (1994). Both incorporated herein by reference. As other examples, the complete genome of AAV-1 is provided in GenBank Accession No. NC_002077 (incorporated herein by reference); the complete genome of AAV-3 is provided in GenBank Accession No. NC_001829 (incorporated herein by reference); the complete genome of AAV-4 is provided in GenBank Accession No. NC_001829 (incorporated herein by reference); the AAV-5 genome is provided in GenBank Accession No. AF085716 (incorporated herein by reference); the complete genome of AAV-6 is provided in GenBank Accession No. NC_001862 (incorporated herein by reference); at least portions of AAV-7 and AAV-8 genomes are provided in GenBank Accession Nos. AX753246 (incorporated herein by reference) and AX753249 (incorporated herein by reference), respectively (see also U.S. Pat. Nos. 7,282,199 and 7,790,449 relating to AAV-8); the AAV-9 genome is provided in Gao et al., J. Virol 78:6381-6388 (2004), incorporated herein by reference; the AAV-10 genome is provided in Mol. Ther. 13(1):67-76 (2006), incorporated herein by reference; and the AAV-11 genome is provided in Virology 330(2):375-383 (2004), incorporated herein by reference. The AAVrh74 serotype is described in Rodino-Klapac et al., J. Trans. Med. 5:45 (2007), incorporated herein by reference.

AAV DNA in the rAAV genomes may be from any AAV serotype for which a recombinant virus can be derived including, but not limited to, AAV serotypes AAV-1, AAV-2, AAV-3, AAV-4, AAV-5, AAV-6, AAV-7, AAV-8, AAV-9, AAV-10, AAV-11, AAV-12, AAV-13, Rh10, Rh74, and AAV-2i8.

Production of pseudotyped rAAV is disclosed in, for example, WO 01/83692 which is incorporated by reference herein in its entirety.

Other types of rAAV variants, for example rAAV with capsid mutations, are also contemplated. See, for example, Marsic et al., Molecular Therapy, 22(11): 1900-1909 (2014). The nucleotide sequences of the genomes of various AAV serotypes are known in the art.

In certain embodiments, to promote skeletal muscle specific expression, AAV1, AAV6, AAV8 or AAVrh.74 may be used.

In certain embodiments, the AAV serotype of the subject AAV vector is AAV9.

Cis-acting sequences directing viral DNA replication (rep), encapsidation/packaging and host cell chromosome integration are contained within the ITRs. Three AAV promoters (named p5, p19, and p40 for their relative map locations) drive the expression of the two AAV internal open reading frames encoding rep and cap genes.

The two rep promoters (p5 and p19), coupled with the differential splicing of the single AAV intron (e.g., at AAV2 nucleotides 2107 and 2227), result in the production of four rep proteins (rep 78, rep 68, rep 52, and rep 40) from the rep gene. Rep proteins possess multiple enzymatic properties that are ultimately responsible for replicating the viral genome.

The cap gene is expressed from the p40 promoter and it encodes the three capsid proteins VP1, VP2, and VP3. Alternative splicing and non-consensus translational start sites are responsible for the production of the three related capsid proteins.

A single consensus polyadenylation site is located at map position 95 of the AAV genome. The life cycle and genetics of AAV are reviewed in Muzyczka, Current Topics in Microbiology and Immunology 158:97-129 (1992).

DNA plasmids of the invention comprise rAAV genomes of the invention. The DNA plasmids are transferred to cells permissible for infection with a helper virus of AAV (e.g., adenovirus, El-deleted adenovirus or herpes virus) for assembly of the rAAV genome into infectious viral particles. Techniques to produce rAAV particles, in which an AAV genome to be packaged, rep and cap genes, and helper virus functions are provided to a cell, are standard in the art. Production of rAAV requires that the following components are present within a single cell (denoted herein as a packaging cell): a rAAV genome, AAV rep and cap genes separate from (i.e., not in) the rAAV genome, and helper virus functions. The AAV rep and cap genes may be from any AAV serotype for which recombinant virus can be derived and may be from a different AAV serotype than the rAAV genome ITRs, including, but not limited to, AAV serotypes AAV-1, AAV-2, AAV-3, AAV-4, AAV-5, AAV-6, AAV-7, AAVrh.74, AAV-8, AAV-9, AAV-10, AAV-11, AAV-12 and AAV-13.

A method of generating a packaging cell is to create a cell line that stably expresses all the necessary components for AAV particle production. For example, a plasmid (or multiple plasmids) comprising a rAAV genome lacking AAV rep and cap genes, AAV rep and cap genes separate from the rAAV genome, and a selectable marker, such as a neomycin resistance gene, are integrated into the genome of a cell. AAV genomes have been introduced into bacterial plasmids by procedures such as GC tailing (Samulski et al., Proc. Natl. Acad. Sci. U.S.A. 79:2077-2081, 1982), addition of synthetic linkers containing restriction endonuclease cleavage sites (Laughlin et al., Gene 23:65-73, 1983) or by direct, blunt-end ligation (Senapathy & Carter, J. Biol. Chem. 259:4661-4666, 1984). The packaging cell line is then infected with a helper virus such as adenovirus. The advantages of this method are that the cells are selectable and are suitable for large-scale production of rAAV.

Other examples of suitable methods employ adenovirus or baculovirus rather than plasmids to introduce rAAV genomes and/or rep and cap genes into packaging cells.

General principles of rAAV production are reviewed in, for example, Carter, Current Opinions in Biotechnology 1533-1539, 1992; and Muzyczka, Curr. Topics in Microbial. and Immunol. 158:97-129, 1992). Various approaches are described in Ratschin et al., Mol. Cell. Biol. 4:2072, 1984; Hermonat et al., Proc. Natl. Acad. Sci. U.S.A. 81:6466, 1984; Tratschin et al., Mol. Cell. Biol. 5:3251, 1985; McLaughlin et al., J. Virol. 62: 1963, 1988; and Lebkowski et al., Mol. Cell. Biol. 7:349, 1988; Samulski et al., J. Virol. 63:3822-3828, 1989; U.S. Pat. No. 5,173,414; WO 95/13365, and corresponding U.S. Pat. No. 5,658,776; WO95/13392; WO 96/17947; PCT/US98/18600; WO 97/09441 (PCT/US96/14423); WO 97/08298 (PCT/US96/13872); WO 97/21825 (PCT/US96/20777); WO 97/06243 (PCT/FR96/01064); WO 99/11764; Perrin et al., Vaccine 13:1244-1250, 1995; Paul et al., Human Gene Therapy 4:609-615, 1993; Clark et al., Gene Therapy 3:1124-1132, 1996; U.S. Pat. Nos. 5,786,211; 5,871,982; and 6,258,595. The foregoing documents are hereby incorporated by reference in their entirety herein, with particular emphasis on those sections of the documents relating to rAAV production.

In certain embodiments, the AAV vectors of the invention are produced according to the method described in Adamson-Small et al. (Molecular Therapy—Methods & Clinical Development (2016) 3, 16031; doi:10.1038/mtm.2016.31, incorporated herein by reference), a scalable method for the production of high-titer and high quality adeno-associated type 9 vectors using the HSV platform. It is a complete herpes simplex virus (HSV)-based production and purification process capable of generating greater than 1×10¹⁴ rAAV9 vector genomes per 10-layer CellSTACK of HEK 293 producer cells, or greater than 1×10⁵ vector genome per cell, in a final, fully purified product. This represents a 5- to 10-fold increase over transfection-based methods. In addition, rAAV vectors produced by this method demonstrated improved biological characteristics when compared to transfection-based production, including increased infectivity as shown by higher transducing unit-to-vector genome ratios and decreased total capsid protein amounts, shown by lower empty-to-full ratios. This method can also be readily adapted to large-scale good laboratory practice (GLP) and good manufacturing practice (GMP) production of rAAV9 vectors to enable preclinical and clinical studies and provide a platform to build on toward late-phases and commercial production. Although AAV9 was used in the study, this method is likely extendable to other serotypes and should bridge the gap between preclinical research, early phase clinical studies, and large-scale, worldwide development of gene therapy based-drugs for genetic diseases and disorders.

The invention thus provides packaging cells that produce infectious rAAV. In one embodiment, packaging cells may be stably transformed cancer cells such as HeLa cells, 293 cells and PerC.6 cells (a cognate 293 line). In another embodiment, packaging cells are cells that are not transformed cancer cells, such as low passage 293 cells (human fetal kidney cells transformed with El of adenovirus), MRC-5 cells (human fetal fibroblasts), WI-38 cells (human fetal fibroblasts), Vero cells (monkey kidney cells) and FRhL-2 cells (rhesus fetal lung cells).

Recombinant AAV (i.e., infectious encapsidated rAAV particles) of the invention comprise a rAAV genome. In exemplary embodiments, the genomes of both rAAV lack AAV rep and cap DNA, that is, there is no AAV rep or cap DNA between the ITRs of the genomes. Examples of rAAV that may be constructed to comprise the nucleic acid molecules of the invention are set out in International Patent Application No. PCT/US2012/047999 (WO 2013/016352) incorporated by reference herein in its entirety.

The rAAV may be purified by methods standard in the art such as by column chromatography or cesium chloride gradients. Methods for purifying rAAV vectors from helper virus are known in the art and include methods disclosed in, for example, Clark et al., Hum. Gene Ther. 10(6):1031-1039, 1999; Schenpp and Clark, Methods Mol. Med. 69:427-443, 2002; U.S. Pat. No. 6,566,118 and WO 98/09657.

Tropism of the AAV viral vector can be selected partly based on the intended target organ or tissue in which the GOIs are to be expressed. The chart below gives a summary of the tropism of selected AAV serotypes, indicating the optimal serotype(s) for transduction of a given organ.

| Tissue | Optimal Serotype |
|---|---|
| CNS | AAV1, AAV2, AAV4, AAV5, AAV8, AAV9 |
| Heart | AAV1, AAV8, AAV9 |
| Kidney | AAV2 |
| Liver | AAV7, AAV8, AAV9 |
| Lung | AAV4, AAV5, AAV6, AAV9 |
| Pancreas | AAV8 |
| Photoreceptor Cells | AAV2, AAV5, AAV8 |
| RPE (Retinal Pigment | AAV1, AAV2, AAV4, AAV5, AAV8 |
| Epithelium) | |
| Skeletal Muscle | AAV1, AAV6, AAV7, AAV8, AAV9 |

For example, AAVpo1 was isolated from pigs and found to efficiently transduce muscle following direct intramuscular injection in mice. It is useful for muscle gene therapies in general due to its ability to robustly transduce all major muscle tissues, including heart and diaphragm, from peripheral infusion.

In certain embodiment, tropism of AAV is further refined through pseudotyping, by mixing capsids and genomes from different AAV serotypes. For example, AAV2/5 indicates a virus containing the genome of serotype 2 packaged in the capsid from serotype 5. The pseudotyped viruses may have improve transduction efficiency, as well as alter tropism.

For example, AAV2/5 targets neurons that are not efficiently transduced by AAV2/2, and is distributed more widely in the brain, indicating improved transduction efficiency. Many of these hybrid viruses have been well characterized and may be preferred over standard viruses for in vivo applications.

In certain embodiment, tropism is further refined by using recombinantly generated hybrid capsids derived from multiple different serotypes. One common example is AAV-DJ, which contains a hybrid capsid derived from eight serotypes. AAV-DJ displays a higher transduction efficiency in vitro than any wild type serotype, and it displays very high infectivity across a broad range of cell types in vivo. The mutant AAV-DJ8 displays the properties of AAV-DJ, but with enhanced brain uptake.

Another engineered AAV-PHP.B family of AAV efficiently delivers gene throughout the CNS, and can be used for gene therapy that requires CNS delivery.

Davidsson et al. (PNAS 116(52):27053-27062, 2019) recently described a so-called BRAVE (barcoded rational AAV vector evolution) approach that enables efficient selection of engineered capsid structures on a large scale using only a single screening round in vivo. Using the BRAVE approach and hidden Markov model-based clustering, the authors presented 25 synthetic capsid variants with refined properties, such as retrograde axonal transport in specific subtypes of neurons, as shown for both rodent and human dopaminergic neurons.

On the other hand, Herrmann et al. (ACS Synth. Biol. 8(1):194-206, 2019) described a particularly powerful technology for breeding novel vectors with improved properties—DNA family shuffling—which generates chimeric capsids by homology-driven DNA recombination.

In certain embodiments, a capsid of the subject viral vector is surfaced-engineered for selected and cell-type specific gene delivery. For example, Buchholz et al. (Trends Biotechnol. 33(12):777-790, 2015) disclose that gene vectors based on lentiviruses or adeno-associated viruses can be engineered, such that they use a cell surface marker of choice for cell entry instead of their natural receptors. Binding to the surface marker is mediated by a targeting ligand displayed on the vector particle surface, which can be a peptide, single-chain antibody, or designed ankyrin repeat protein. Examples include vectors that deliver genes to specialized endothelial cells or lymphocytes, tumor cells, or particular cells of the nervous system.

### Additional Coding Sequences

For muscular dystrophy treatment, in addition to the coding sequence for a dystrophin protein, such as microD5, the recombinant vector of the invention also comprises one or more additional coding sequences for targeting gene(s) in one of the secondary complications/secondary cascades associated with or resulting from loss of dystrophin.

In certain embodiments, the vector of the invention encodes an exon-skipping antisense sequence that can correct specific dystrophin gene mutations.

For example, the exon-skipping antisense sequence induces skipping of specific exons during pre-messenger RNA (pre-mRNA) splicing of a defective dystrophin gene in the subject, resulting in restoration of the reading frame and partial production of an internally truncated protein, similar to the dystrophin protein expression seen in Becker muscular dystrophy.

In certain embodiments, the exon-skipping antisense sequence skips or splices out a frame-disrupting exon (mutated exon) and/or a neighboring exon to restore the correct transcriptional reading frame, and to produce a shorter but functional dystrophin protein.

In certain embodiments, the exon-skipping antisense sequence induces single exon skipping. In certain embodiments, the exon-skipping antisense sequence induces multiple exon skipping, such as skipping of one or more of, or all of exons 45-55 (i.e., native exons 44 is joined directly to exon 56). For example, 11 antisense sequences may be used together to skip all 11 exons including exons 45-55. A cocktail of 10 AONs was used in the mdx52 mouse model (with deletion of exon 52) to induce skipping of exon 45-51 and 53-55, thus restoring functional dystrophin expression.

In certain embodiments, the exon-skipping antisense sequence induces skipping of exon 51 in a dystrophin pre-mRNA. Successful skipping of exon 51 can in theory treat about 14% of all DMD patients.

In certain embodiments, the exon-skipping antisense sequence targets an exonic splice enhancer (ESE) site in exon 51 of dystrophin gene, thus causing a skip of exon 51 and producing a truncated but partially functional dystrophin protein.

In certain embodiments, the exon-skipping antisense sequence induces skipping of one or more of exons 44,45, and 53.

In certain embodiments, the exon-skipping antisense sequence targets the same target sequence as that of casimersen (exon 45), NS-065/NCNP-01 or golodirsen (exon 53), or eteplirsen or Exondys 51 (exon 51).

In certain embodiments, the exon-skipping antisense sequence targets a cryptic splicing donor and/or acceptor site in the mutated FCMD/FKTN gene in a Fukuyama congenital muscular dystrophy (FCMD) patient to restore correct exon 10 splicing.

Fukuyama congenital muscular dystrophy (FCMD) is a rare autosomal recessive disease and the second prevalent form of childhood muscular dystrophy in Japan. The gene responsible for FCMD (FCMD, also known as FKTN) encodes the protein fukutin, which is a putative glycosyltransferase and glycosylates α-dystroglycan, a member of the dystrophin-associated glycoprotein complex (DAGC). The pathogenesis of FCMD is caused by an ancestral insertion of SINE-VNTR-Alu(SVA) retrotransposon into the 3′-untranslated region (UTR) of the fukutin gene, leading to the activation of a new, cryptic splice donor in exon 10, and a new, cryptic splice acceptor in the SVA insertion site, thus inducing aberrant mRNA splicing between the cryptic donor and acceptor sites. The result is a premature truncation of exon 10 of FCMD. In FCMD patient cells and model mice in vivo, it has been shown that a cocktail of three vivo-PMOs targeting the cryptic splice modulating regions prevented pathogenic SVA exon trapping and restored normal FKTN protein levels and O-glycosylation of α-dystroglycan.

In certain embodiments, the antisense sequence targets a pathological expansion of 3- or 4-nucleotide repeats, such as a CTC triplet repeat in the 3′-UTR region of the DMPK gene in DM1 patients, or a CCTG repeat in the first intron of the CNBP gene in DM2 patients.

Myotonic dystrophy (DM) is the most common form of muscular dystrophy in adulthood. It is an autosomal dominant disease that can be categorized into myotonic dystrophy type 1 (DM1) and myotonic dystrophy type 2 (DM2). DM1 is caused by a pathological expansion of CTC triplet in 3′-UTR region of the Dystrophia Myotonica Protein Kinase (DMPK) gene, while DM2 is caused by a pathological expansion of CCTG tract in the first intron of the CCHC-type zinc finger, nucleic acid binding protein gene (CNBP). RNA gain-of-function toxicity, arising from transcribed RNA aggregates with expanded repeats, leads to aberrant splicing (spliceopathy). Aggregates of toxic RNA disrupt the function of alternative splicing regulators such as Muscleblind-like (MBNL) protein and CUG-binding protein 1 (CUGBP1), by sequestering and depleting the former within the nuclear RNA foci, and increasing the expression and phosphorylation of the latter in DM1. Alterations in the function of MBNL and CUGBP1 proteins lead to aberrant splicing in pre-mRNAs of target genes, namely insulin receptor (INSR), the muscle chloride channel (CLCN1), bridging integrator-1 (BIN1), and dystrophin (DMD), which are respectively associated with insulin resistance, myotonia, muscle weakness, and dystrophic muscle processes (all typical symptoms of myotonic dystrophy).

Thus an expanded CUG repeat in the DMPK gene sequesters MBNL1 protein and causes aberrant splicing in several downstream genes, thereby causing DM1 phenotype. Meanwhile, antisense oligonucleotides can be used to target such expanded repeats of mutant transcripts for RNase-mediated degradation, thereby restoring splicing of downstream genes. A 2′-O-methoxyethyl gapmer AON has been used to target the degradation of expanded CUG by RNase H in mutant RNA transcripts, resulting in a reduction of mutant mRNA transcripts and restored protein expression.

In certain embodiments, the exon-skipping antisense sequence leads to skipping of exon 7A in CLCN1 gene in a DM1 patient.

DM1 can also be treated by correcting aberrant splicing of chloride channel 1 (CLCN1), as this gene causes myotonia in DM1 patients. Using PMOs (phosphorodiamidate morpholino oligomer) with bubble liposomes through ultrasound exposure to enhance delivery of PMOs into muscles of DM1 mice (HSALR), skipping of exon 7A of CLCN1 was achieved in vivo, resulting in ameliorated myotonia and Clcn1 protein expression in skeletal muscles.

In certain embodiments, the exon-skipping antisense sequence targets exons 17, 32, 35, 36, and/or 42 of the DYSF gene, preferably exon 32 and/or 36, for exon skipping in a dysferlinopathy (e.g., LGMD2B or MM) patient with a DYSF mutation.

Dysferlinopathy is an umbrella term that encompasses muscular dystrophies caused by mutations in the dysferlin (DYSF) gene. Dysferlin gene encodes a sarcolemmal protein required for repairing muscle membrane damage. It consists of calcium-dependent C2 lipid binding domains and a vital transmembrane domain. There are two common dysferlinopathies—limb-girdle muscular dystrophy type 2B (LGMD2B) and Miyoshi myopathy (MM), both have clinically distinct phenotypes and an autosomal recessive inheritance. LGMD2B is characterized by proximal muscle weakness, while MM is characterized by distal muscle weakness. Initial clinical phenotypes of LGMD2B and MM are distinct. However, as the disease progresses, the clinical presentations for both conditions overlap, becoming more similar, and patients experience muscle weakness in both proximal and distal limbs. Dysferlin-deficient muscle fibers have a defect in membrane repair.

Dysferlinopathies can be treated by exon skipping using antisense oligonucleotides, partly due to the observed mild phenotype in a patient with only 10% wild-type level expression of a truncated mutant DYSF protein. Specifically, in an LGMD2B case of a compound heterozygous female patient, the patient harbored one null allele and a DYSF branch point mutation on the other allele in intron 31. A natural in-frame skipping of exon 32 resulted in a truncated dysferlin protein expressed at about 10% that of the wild type levels, which was sufficient to partially complement the null mutation. The patient exhibited mild symptoms, and was ambulant at age 70. Recently, it has been shown that exon 32 skipping in patient cells resulted in quasi-dysferlin expression levels, which rescued membrane repair in treated cells that were subject to hypo-osmotic pressure and sarcolemmal localized laser injury in vitro.

In certain embodiments, the exon-skipping antisense sequence targets exon 4 of the LAMA2 gene, for exon skipping in a merosin-deficient congenital muscular dystrophy type 1A (MDC1A) patient with a LAMA2 mutation. In certain embodiments, exon skipping results in restored expression of the C-terminal G-domain (exons 45-64), particularly G4 and G5 that are most important for mediating interaction with α-dystroglycan.

Merosin-deficient congenital muscular dystrophy type 1A (MDC1A) is caused by mutations in the 65-exon LAMA2 gene that results in a complete or partial deficiency in laminin-α2 chain expression. Laminin-α2 chain, together with beta1 (β1), and gamma1 (γ1) chains, are parts of the heterotrimeric laminin isoform known as Laminin-211 or merosin, which is expressed particularly in the basement membranes of skeletal muscles, including the neuromuscular junction and Schwann cells (peripheral nerves). Laminin-α2 interacts with the dystrophin-dystroglycan complex (DGC), mediating cell signaling, adhesion, and tissue integrity in skeletal muscles and peripheral nerves. Although not always the case, the partial expression of laminin-α2 causes milder MDC1A, while complete absence of laminin-α2 causes severe MDC1A. The C-terminal G-domain (exons 45-64), particularly G4 and G5, are most important for mediating interaction with α-dystroglycan. Mutations eliminating G4 and G5 is associated with severe phenotypes even in the presence of partial truncated laminin-α2 expression.

Exon-skipping has been explored for treating MDC1A, in that PMO-mediated exon 4 skipping corrected the open reading frame, resulting in the recovery of a truncated laminin-α2 chain and a slightly extended patient life span.

In certain embodiments, the exon-skipping antisense sequence induces skipping of exons 4-7 of the most common Δ-521T mutation in the LGMD2C/SGCG gene, and restoration of the reading frame to generate an internally truncated SGCG protein, for treating a limb-girdle muscular dystrophy type 2C patient with a Δ-521T SGCG mutation. In certain embodiments, exon skipping results in restored expression of the internally truncated SGCG protein that retains the intracellular, transmembrane, and extreme carboxy-terminus of the wild-type SGCG protein.

Dystrophin-associated protein (DAP) is a complex in the muscle cell membrane, the transmembrane components of which link the cytoskeleton to the extracellular matrix in adult muscle fibers, and are essential for the preservation of the integrity of the muscle cell membrane. The sarcoglycan subcomplex within the DGC is composed of 4 single-pass transmembrane subunits: α-, β-, γ-, and δ-sarcoglycan. The α to δ-sarcoglycans gene, namely α (LGMD2D), β (LGMD2E), γ (LGMD2C) and δ (LGMD2F), are expressed predominantly (β) or exclusively (α, γ and δ) in striated muscle. A mutation in any of the four sarcoglycan genes may lead to a secondary deficiency of the other sarcoglycan proteins, presumably due to destabilization of the sarcoglycan complex, leading to sarcoglycanopathies—autosomal recessive limb girdle muscular dystrophies (LGMDs). The disease-causing mutations in the α to δ genes cause disruptions within the dystrophin-associated protein (DAP) complex in the muscle cell membrane.

In human, γ sarcoglycan (LGMD2C) is a protein encoded by the SGCG gene. Severe childhood autosomal recessive muscular dystrophy (SCARMD) is a progressive muscle-wasting disorder that segregates with microsatellite markers at the γ-sarcoglycan gene. Mutations in the γ-sarcoglycan gene were first described in the Maghreb countries of North Africa, where γ-sarcoglycanopathy has a higher than usual incidence. One of the most common mutation in LGMD 2C patients, Δ-521T, is a deletion of a thymine from a string of 5 thymines at nucleotide bases 521-525 in exon 6 of the γ-sarcoglycan gene. This mutation shifts the reading frame and results in the absence of γ-sarcoglycan protein and secondary reduction of β- and δ-sarcoglycans, thus causing a severe phenotype. The mutation occurs both in the Maghreb population and in other countries.

Exon-skipping has been explored for treating LGMD2C with the Δ-521T mutation, in that the resulting internally truncated SGCG protein provided functional and pathological benefits to correct the loss of γ-sarcoglycan in a Drosophila model, in heterologous cell expression studies, and in transgenic mice lacking γ-sarcoglycan. A cellular model of human muscle disease was also generated to show that multiple exon skipping could be induced in RNA that encodes a mutant human γ-sarcoglycan.

In certain embodiments, the vector of the invention encodes an antisense sequence, or an RNAi sequence (siRNA, shRNA, miRNA etc.), that antagonizes the function of sarcolipin (SLN). In certain embodiments, the vector of the invention encodes an shRNA that antagonizes the function of sarcolipin (shSLN).

Exemplary shSLN sequences include those disclosed in FIGS. 9 and 10 of PCT/US2019/065718, filed on Dec. 11, 2019 (e.g., the underlined sequences in FIG. 9, and the highlighted sequences in FIG. 10). Additional exemplary shSLN sequences include SEQ ID NOs: 7-11 disclosed in WO2018/136880 (incorporated herein by reference).

Further shSLN sequences can be designed based on any art recognized methods, using the human SLN mRNA sequence shown below.

(SEQ ID NO: 4)

| | |
|---|---|
| 1 | AGACAGCCTG GGAGGGGAGA AGGAGTTGGA GCTCAAGTTG |
| | GAGACAGCGA GGAGAAACCT |
| 61 | GCCATAGCCA GGGTGTGTCT TTGATCCTCT TCAGGAGGTG |
| | AGGAGAAGCC AGAGGTCCTT |
| 121 | GGTGTGCCCT CAGAAATCTG CCTGCAGTTC TCACCAAGCC |
| | GCTGTGAAAA TGGGGATAAA |
| 181 | CACCCGGGAG CTGTTTCTCA ACTTCACTAT TGTCTTGATT |
| | ACGGTTATTC TTATGTGGCT |
| 241 | CCTTGTGAGG TCCTATCAGT ACTGAGAGGC CATGCCATGG |
| | TCCTGGGATT GACTGAGATG |
| 301 | CTCCGGAGCT GCCTGCTCTA TGCCCTGAGA CCCCACTGCT |
| | GTCATTGTCA CAGGATGCCA |
| 361 | TTCTCCATCC GAGGGCACCT GTGACCTGCA CTCACAATAT |
| | CTGCTATGCT GTAGTGCTAG |
| 421 | GATTGATTAT GTGTTCTCCA AAGATGCTGC TCCCAAGGGC |
| | TGCCAAGTGT TTGCCAGGGA |
| 481 | ACGGTAGATT TATTCCCCAA CTCTTAACTG AAAATGTGTT |
| | AGACAAGCCA CAAAGTTAAA |
| 541 | ATTAAACTGG ATTCATGATG ATGTAGGATT GTTACAAGCC |
| | CCTGATCTGT CTCACCACAC |
| 601 | ATCCCTTCAA CCCACACGGT CTGCAACCAA ACTCTAATTC |
| | AACCTGCCAG AAGGAATGTT |
| 661 | AGAGGAAGTC TTTGTCAGCC CTTATAGCTA TCATGTGAAT |
| | AAAGTTAAGT CAACTTCAAA |
| 721 | AA |

In certain embodiments, the vector of the invention encodes an antisense sequence, or an RNAi sequence (siRNA, shRNA, miRNA etc.), that antagonizes the function of one or more target genes, such as an inflammatory gene.

The IκB kinase/nuclear factor-kappa B (NF-κB) signaling is persistently elevated in immune cells and regenerative muscle fibers in both animal models and patients with DMD. In addition, activators of NF-κB such as TNF-α and IL-1 and IL-6 are upregulated in DMD muscles. Thus, inhibiting the NF-κB signaling cascade components, such as NF-κB itself, its upstream activators and the downstream inflammatory cytokines, are beneficial for treating the subject patients in conjunction with replacing/repairing a defective dystrophin gene.

Thus in certain embodiments, the vector of the invention encodes an antisense sequence, or an RNAi sequence (siRNA, shRNA, miRNA etc.), that antagonizes the function of one or more inflammatory genes, such as NF-κB, TNF-α, IL-1 (IL-1β), IL-6, Receptor activator of NF-κB (RANK), and Toll-like receptors (TLRs).

In certain embodiments, the vector of the invention encodes an antisense sequence, or an RNAi sequence (siRNA, shRNA, miRNA etc.), that antagonizes the function of a histone deacetylase, such as HDAC2. In DMD, the absence of dystrophin at the sarcolemma delocalizes and downregulates nitric oxide synthase (nNOS), which alters S-nitrosylation of HDAC2 and its chromatin association. In the dystrophin-deficient mdx mice, which are defective for the NO pathway, the activity of HDAC2 resulted to be specifically increased. In contrast, rescue of nNOS expression in mdx animals ameliorated the dystrophic phenotype. In addition, deacetylase inhibitors conferred a strong morphofunctional benefit to dystrophic muscle fibers. Indeed, givinostat, a histone deacetylase inhibitor, is under evaluation as potential disease-modifying treatment for DMD. Data indicates that, in both murine and human dystrophic cells, the absence of dystrophin correlates with HDAC2 binding to a specific subset of miRNAs (see below), while upon dystrophin rescue HDAC2 is released from these promoters.

In certain embodiments, the vector of the invention encodes an antisense sequence, an RNAi sequence (siRNA, shRNA, miRNA etc.), or a microRNA, that antagonizes the function of TGF-β, or connective tissue growth factor (CTGF). Elevated levels of TGF-β in muscular dystrophies stimulate fibrosis and impair muscle regeneration by blocking the activation of satellite cells. Anti-fibrotic agents have been tested in murine models of muscular dystrophy, including losartan, an angiotensin II-type 1 receptor blocker that reduces the expression of TGF-β. HT-100 (halofuginone) has also been shown to prevent fibrosis via the TGF-β/Smad3 pathway in muscular dystrophies. Meanwhile, FG-3019, a fully human monoclonal antibody that interferes with the action of connective tissue growth factor, a central mediator in the pathogenesis of fibrosis, has been evaluated in an open-label phase 2 trial in patients with idiopathic pulmonary fibrosis (IPF).

In certain embodiments, the vector of the invention encodes a microRNA (miR), such as miR-1, miR-29c, miR-30c, miR-133, and/or miR-206. The differential HDAC2 nitrosylation state in Duchenne versus wild-type conditions deregulates the expression of a specific subset of microRNA genes. Several circuitries controlled by the identified microRNAs, such as the one linking miR-1 to the G6PD enzyme and the redox state of cell, or miR-29 to extracellular proteins and the fibrotic process, explain some of the DMD pathogenetic traits. The muscle-specific (myomiR) miR-1 and miR-133, and the ubiquitous miR-29c and miR-30c, downregulated in mdx, recovered toward wildtype levels in exon-skipping-treated animals. According to the mdx model, when dystrophin synthesis was restored via exon skipping, the levels of miR-1, miR-133a, miR-29c, miR-30c, and miR-206 increased, while miR-23a expression did not change.

In certain embodiments, the vector of the invention encodes a microRNA inhibitor, which inhibits the function of a microRNA upregulated in DMD or its related diseases. For example, the inflammatory miR-223 expression level is upregulated in mdx mice muscles, and is downregulated in exon-skipping-treated mice. Its decrease is consistent with the observed amelioration of the inflammatory state of the muscle, due to dystrophin rescue by exon-skipping.

The mdx animals undergo extensive fibrotic degeneration, and miR-29 has been shown to target mRNAs of crucial factors involved in fibrotic degeneration, such as collagens, elastin, and structural components of the extracellular matrix. In mdx mice, miR-29 is poorly expressed, and the mRNAs for collagen (COL1A1) and elastin (ELN) were upregulated. Thus expression of miR-29c alleviates fibrotic degeneration in DMD patients, partly through downregulating collagen and elastin expression, and pathological extracellular matrix modification associated with collagen and elastin expression.

In certain embodiments, the vector of the invention encodes an antisense sequence, or an RNAi sequence (siRNA, shRNA, miRNA etc.), that antagonizes the function of G6PD (Glucose-6-phosphate dehydrogenase). One important issue in dystrophic muscles is their susceptibility and response to oxidative stress suggested to be involved in disease progression. G6PD is a cytosolic enzyme in the pentose phosphate pathway that supplies reducing energy to cells by maintaining the level of NADPH, which in turn ensures high ratio between reduced and oxidized glutathione (GSH/GSSG), GSH being the major antioxidant molecule that protects cells against oxidative damage. G6PD mRNA is deregulated in mdx muscles. It contains in its 3′-UTR region three putative binding sites for the miR-1 family, and miR-1 and miR-206 are able to repress G6PD expression. Indeed, there is an inverse correlation between G6PD and miR-1 expression: in vitro differentiation of C2 myoblasts showed that the increase in miR-1 levels correlated with decrease of G6PD protein, mRNA levels, and GSH/GSSG ratio. In mdx mice, where miR-1 is downregulated, G6PD was detected at higher levels than in WT muscles, whereas in exon-skipping-treated mdx, in which miR-1 resumes, the amount of G6PD was reduced. Notably, in mdx mice, increase in G6PD levels was accompanied by a decrease in GSH/GSSG ratio.

In certain embodiments, the vector of the invention encodes an antisense sequence, or an RNAi sequence (siRNA, shRNA, miRNA etc.), that antagonizes the function of myostatin. Myostatin is a negative regulator of muscle mass. Inhibition or blockade of endogenous myostatin compensates for the severe muscle wasting common in many types of muscular dystrophies including DMD. A myostatin blocking antibody, MYO-029, is in clinical trial for adult subjects with BMD and other dystrophies. Other clinical trials using myostatin inhibitors such as follistatin and PF-06252616 (NCT02310764) and BMS-986089 have also been conducted.

In certain embodiments, the vector of the invention encodes an antisense sequence, or an RNAi sequence (siRNA, shRNA, miRNA etc.), that antagonizes the function of phosphodiesterase-5 (PED-5) or ACE, or VEGF decoy-receptor type 1 (VEGFR-1 or Flt-1). Loss of dystrophin leads to displacement of neuronal nitric oxide synthase and reduction of muscle-derived nitric oxide to the microvasculature, resulting in functional muscle ischemia and further muscle injury. Thus several inhibitors of phosphodiesterase-5 or ACE, or VEGF decoy-receptor type 1 (VEGFR-1 or Flt-1), have been tested as part of the strategies to increase blood flow to muscles, include pharmaceutical inhibition of either phosphodiesterase-5 or ACE.

In certain embodiments, the vector of the invention encodes an antisense sequence, or an RNAi sequence (siRNA, shRNA, miRNA etc.), that antagonizes the function of hematopoietic prostaglandin D synthase (HPGDS). Prostaglandin D2 (PGD2) is produced by various inflammatory cells, and hematopoietic PGD synthase (HPGDS) is shown to be expressed in necrotic muscle of DMD patients. The administration of an HPGDS inhibitor decreased the urinary excretion of tetranor-PGDM, a urinary metabolite of PGD2, and suppressed myonecrosis in a mdx mouse model of DMD. TAS-205, a novel HPGDS inhibitor, has been evaluated for DMD treatment in clinical trial.

### RNAi and Antisense Design

In RNA interference (RNAi), short RNA molecules are created that are complimentary and bind to endogenous target mRNA. Such binding leads to functional inactivation of the target mRNA, including degradation of the target mRNA.

The RNAi pathway is found in many eukaryotes, including plants and animals, and is initiated in the cytoplasm by the enzyme Dicer, which cleaves long double-stranded RNA (dsRNA) or small hairpin RNAs (shRNA) molecules into short double-stranded fragments of ˜21 nucleotide siRNAs. Each siRNA is then unwound into two single-stranded RNAs (ssRNAs), the passenger strand and the guide strand. The passenger strand is degraded and the guide strand is incorporated into the RNA-induced silencing complex (RISC). The most well-studied outcome is post-transcriptional gene silencing, which occurs when the guide strand pairs with a complementary sequence in an mRNA molecule and induces cleavage by Argonaute 2 (Ago2), the catalytic component of the RISC. In some organisms, this process spreads systemically, despite the initially limited molar concentrations of siRNA.

Other than the siRNA and shRNA, another type of small RNA molecules that are central to RNA interference is microRNA (miRNA).

MicroRNAs are genomically encoded non-coding RNAs that help regulate gene expression, particularly during development. Mature miRNAs are structurally similar to siRNAs, but they must first undergo extensive post-transcriptional modification before reaching maturity. An miRNA is expressed from a much longer RNA-coding gene as a primary transcript known as a pri-miRNA, which is then processed in the cell nucleus to a 70-nucleotide stem-loop structure called pre-miRNA, by the microprocessor complex consisting of an RNase III enzyme Drosha and a dsRNA-binding protein DGCR8. Upon transporting this pre-miRNA into the cytosol, its dsRNA portion is bound and cleaved by Dicer to produce the mature miRNA molecule, which two strands can be separated into a passenger strand and a guide strand. The miRNA guide strand, like the siRNA guide strand, can be integrated into the same RISC complex.

Thus, the two dsRNA pathways, miRNA and siRNA/shRNA, both require processing of a precursor molecule (pri-miRNA, pre-miRNA, and dsRNA or shRNA) with a backbone sequence in order to generate the mature functional guide strand for miRNA or siRNA, and both pathways eventually converge at the RISC complex.

After integration into the RISC, siRNAs base-pair to their target mRNA and cleave it, thereby preventing it from being used as a translation template. Differently from siRNA, however, a miRNA-loaded RISC complex scans cytoplasmic mRNAs for potential complementarity. Instead of destructive cleavage (by Ago2), miRNAs target the 3′-UTR regions of mRNAs where they typically bind with imperfect complementarity, thus blocking the access of ribosomes for translation.

siRNAs differ from miRNAs in that miRNAs, especially those in animals, typically have incomplete base pairing to a target and inhibit the translation of many different mRNAs with similar sequences. In contrast, siRNAs typically base-pair perfectly and induce mRNA cleavage only in a single, specific target.

Historically, siRNA and shRNA have been used in RNAi applications. siRNA is typically a double-stranded RNA molecules, 20-25 nucleotides in length. siRNA inhibits the target mRNA transiently until they are also degraded within the cell. shRNA is typically ˜80 base pairs in length, that include a region of internal hybridization that creates a hairpin structure. As described previously, shRNA molecules are processed within the cell to form siRNA, which in turn knock down gene expression. One benefit of shRNA is that they can be incorporated into plasmid vectors and integrated into genomic DNA for longer-term or stable expression, and thus longer knockdown of the target mRNA.

shRNAs design is commercially available. For example, Cellecta offers RNAi screening service against any target gene (e.g., all 19,276 protein-encoding human genes) using the Human Genome-Wide shRNA Library or Mouse DECIPHER shRNA Library (which targets about 10,000 mouse genes). ThermoFisher Scientific provides Silencer Select siRNA (classic 21-mers) from Ambion, which, according to the manufacturer, incorporates the latest improvements in siRNA design, off-target effect prediction algorithms, and chemistry.

ThermoFisher Scientific also provides Ambion® Pre-miR™ miRNA Precursor Molecules that are small, chemically modified double-stranded RNA molecules designed to mimic endogenous mature miRNAs. Use of such Pre-miR miRNA Precursors enable miRNA functional analysis by up-regulation of miRNA activity, and can be used in miRNA target site identification and validation, screening for miRNAs that regulate the expression of a target gene, and screening for miRNAs that affect a function of the target gene (such as SLN) or a cellular process.

ThermoFisher Scientific further provides Ambion® Anti-miR™ miRNA Inhibitors, which are chemically modified, single stranded nucleic acids designed to specifically bind to and inhibit endogenous microRNA (miRNA) molecules.

Antisense sequence design is also commercially available from a number of commercial and public sources, such as IDT (Integrated DNA Technologies) and GenLink. Design considerations may include oligo length, secondary/tertiary structure in the target mRNA, protein-binding sites on target mRNA, presence of CG motifs in either the target mRNA or the antisense oligo, formation of tetraplexes in antisense oligo, and the presence of antisense activity-increasing or -decreasing motifs.

Exon skipping antisense oligo design is known in the art. See, for example, Camilla Bernardini (ed.), Duchenne Muscular Dystrophy: Methods and Protocols, Methods in Molecular Biology, vol. 1687, DOI 10.1007/978-1-4939-7374-3_10, Chapter 10 by Shimo et al., published by Springer Science+Business Media LLC, 2018), which discuss in detail the design of effective exon-skipping oligonucleotides, taking into consideration factors such as the selection of target sites, the length of the oligoes, the oligo chemistry, and the melting temperature versus the RNA strand, etc. Also discussed is the use of a cocktail of antisense oligoes to skip multiples exons. The specific genes and muscular dystrophies covered include: DMD (Duchenne muscular dystrophy), LAMA2 (merosine-deficient CMD, DYSF (dysferlinopathy, FKTN (Fukuyama CMD), DMPK (myotonic dystrophy, and SGCG (LGMD2C). The entire content is incorporated herein by reference.

For example, protein/gene sequences and mutations thereof in the affected disease genes are publically available from NCBI and the Leiden muscular dystrophy pages online. Potential target sites for efficient exon skipping can be obtained by using the human splicing finder website at www dot umd dot be slash HSF. Secondary structure of the target mRNA can be evaluated using, e.g., the mfod web server at the Albany dot edu website. The length of the oligoes normally can be 8-30 mer. Oligo GC content calculation is available at OligoCalc website at the Northwestern University server. Search for any off-target sequences can be done using the GGGenome website. Melting temperature of the oligoes can be estimated by LNA oligo prediction tool or OligoAnalyzer 3.1 software at sg dot idtdna dot com.

Enhanced Guide Strand Generation for RNAi (miR, siRNA, & shRNA)

In certain embodiments, the coding sequence encodes an RNAi reagent, such as miR, siRNA, or shRNA.

In certain embodiments, for miR and/or shRNA/siRNA design, the wild-type backbone sequence from which a mature miR or a mature siRNA is generated can be modified to enhance guide strand generation and minimize/eliminate passenger strand production. Since both strands of a mature miR/siRNA/shRNA (after cleavage) can in theory be incorporated into the RISC complex and become guide strand for RNAi, it is advantageous to selectively enhance the utilization of the designed guide strand and minimize the utilization of the largely complementary passenger strand in the RISK complex, in order to reduce or minimize, e.g., off-target effect (e.g., due to the cleavage of unintended target sequences when the passenger strand is loaded into the RISC.

One approach that can be used to achieve this goal (enhance leading strand generation and minimize/eliminate passenger strand production) is through using a hybrid construct in which the designed mature miR/siRNA/shRNA sequences comprising the desired guide strand are embedded inside the backbone sequences of other miR sequences which favor the generation of the guide strand and disfavor the production of the passenger strand.

This principle is illustrated in the design of a few modified miR-29c constructs and shSLN constructs, though the same principle that can be readily adopted for other RNAi reagents targeting any other sequences.

For all designs illustrated below, the adopted design strategy includes engineering flanking backbone sequences, loop sequences, and passenger strand nucleotide sequences, in order to preserve the 2D and 3D structure of the natural backbone sequence. In this context, for miRNA/shRNA designs, 2D/3D structure of the natural backbone sequence refers largely to the distances between stem loop and the flanking backbone polynucleotide sequences, the structure of the central stem, the location and/or sizes of the bulges, the presence and localization of any internal loops and mismatches within the stem, etc. Certain exemplary 2D structure maps for selected miR-30E, miR-101, and miR-451 backbone sequence-based miR-29c hybrid constructs are provided below as illustration.

A. Hybrid miR-29c with miR-30 Backbone Sequence (29c-M30E)

Fellmann et al. (Cell Rep. 5(6):1704-1713, 2013, incorporated herein by reference) describe a systematic approach to optimize the experimental miR-30 backbone, by identifying a conserved element 3′ of the basal stem as critically required for optimal processing of so-called “shRNAmir”—a synthetic shRNA embedded into endogenous microRNA contexts. The resulting optimized backbone, termed “miR-E,” strongly increased mature shRNA levels and knockdown efficacy. This approach can easily convert existing miR and shRNA reagents to miR-E for generating more effective miR and shRNA.

Applying this technology, 29c-M30E hybrid sequences were generated based on the desired mature miR-29c sequence, and the engineered/optimized miR-30 backbone sequence described in Fellmann et al. This 29c-M30E sequence (see FIG. 29 of PCT/US2019/065718, filed on Dec. 11, 2019 for its predicted 2D structure) has been incorporated into the following subject viral vectors used in the examples below: μDys-29c-M30E-i2, EF1A-29c-M30E, U6-29c-M30E. The following 5′→3′ sequence of 29c-M30E is a continuous sequence artificially separated to different lines to illustrate the different segments of the continuous sequence.

TCGACTTCTTAACCCAACAGAAGGCTCGAGAAGGTATATTGCTGTTGACA
GTGAGCGTAACCGATTTCAAATGGTGCTA TAGTGAAGCCACAGATGTA
TAGCACCATTTGAAATCGGTTATGCCTACTGCCTCGGACTTCAAGGGGCT
AGAATTCGA

Specifically, in the continuous sequence above, the middle line represents the passenger strand sequence, the double underlined loop sequence, and the mature miR-29c guide sequence. Note that the passenger and guide sequences can be reverse complement of each other and can snap back and form a stem-loop structure with the intervening loop sequence. However, it should be noted that perfect reverse complement sequences are not necessary. There can be internal bulges, etc., and therefore the two strands are not necessarily 100% complementary to each other in some cases (see the guide and passenger strands in the last sequence of this subsection). The top line and the bottom line represent the M30E flanking backbone sequence optimized to ensure enhanced production of the guide sequence and to minimize the production of the passenger strand.

In a similar design, an siRNA targeting human SLN is embedded in the same M30E backbone sequence in miR-30E-hSLN-c1 (compare the top and bottom rows of the sequences immediately above and below this paragraph, and the double underlined loop sequence). But the guide and passenger strands are different. This so-called c1-M30E sequence has been incorporated into the following subject viral vectors used in the examples below: c1-M30E-i2, c1-M30E-3UTR, and c1-M30E-pa.

TCGACTTCTTAACCCAACAGAAGGCTCGAGAAGGTATATTGCTGTTGACA
GTGAGCGAACTTCACTATTGTCTTGATTAC TAGTGAAGCCACAGATGTA
GTAATCAAGACAATAGTGAAGTTTGCCTACTGCCTCGGACTTCAAGGGGC
TAGAATTCGA

A similarly designed second siRNA also targeting human SLN is embedded in the same M30E backbone sequence in miR-30E-hSLN-c2 (compare the top and bottom rows of the sequences immediately above and below this paragraph, and the double underlined loop sequence). But the guide and passenger strands are different. This so-called c2-M30E sequence has been incorporated into the following subject viral vectors used in the examples below: c2-M30E-i2, c2-M30E-3UTR, and c2-M30E-pa.

TCGACTTCTTAACCCAACAGAAGGCTCGAGAAGGTATATTGCTGTTGACA
GTGAGCGAACACCCGGGAGCTGTTTCTCAA TAGTGAAGCCACAGATGTA
TTGAGAAACAGCTCCCGGGTGTTTGCCTACTGCCTCGGACTTCAAGGGGC
TAGAATTCGA

The sequence of a modified miR-29c using the natural miR-30 backbone sequence (“M30N”) is also provided below as a comparison. Note the guide strand in this case is 5′ to the loop sequence. This M30N backbone sequence similarly enhanced the production of the guide strand, though to a lesser extent than the M30E backbone sequence in the experimental system tested (data not shown).

GGTTAACCCAACAGAAGGCTAAAGAAGGTATATTGCTGTTGACAGTGAGCGAC
TAGCACCATTTGAAATCGGTTA CTGTGAAGCCACAGATGGG TAACCGATTAAATGGTGCTA
GCTGCCTACTGCCTCGGACTTCAAGGGGCTACTTTAGGA

B. Hybrid miR-29c with miR-101 Backbone Sequence (29c-101)

A different miR-29c hybrid (29c-101, see FIG. 30 of PCT/US2019/065718, filed on Dec. 11, 2019 for its predicted 2D structure) using the miR-101 backbone sequence is illustrated below using the same naming convention herein. Here, the top row and the last two rows represent the backbone sequences of miR-101, while the 2nd row is the mature miR-29c with the passenger strand, loop sequence, and guide strand. This 29c-101 sequence has been incorporated into the following subject viral vectors used in the examples below: μDys-29c-10142, μDys-29c-3UTR-101.

CCACCAGAAAGGATGCCGTTGACCGACACAGTGACTGACAGGCTGCCCTGGCG
AACCGATTTCAAATGGTGCATACC GTCTATTCTAAAGG TAGCACCATTTGAAATCGGTTA
GGATGGCAGCCATCTTACCTTCCATCAGAGGAGCCTCACCGTACCCAGGAAGAAAGAAGGTGAAAGAG
GAATGTGAAACAGGTGGCTGGGA

C. Hybrid miR-29c with miR-155 Backbone Sequence (29c-155)

A different miR-29c hybrid (29c-155) using the miR-155 backbone sequence is illustrated below using the same naming convention herein. Here, the top row and the bottom row represent the flanking backbone sequences of miR-155, while the 2nd row is the mature miR-29c with the guide strand, loop sequence, and passenger strand. This 29c-155 sequence has been incorporated into the following subject viral vector used in the examples below: EF1A-29c-155.

CCTGGAGGCTTGCTGAAGGCTGTATGCTG
TAGCACCATTTGAAATCGGTTA TTTTGGCCTCTGACTGA TGACCGCTGGAATGGTGCTA
CAGGACACAAGGCCTGTTACTAGCACTCACATGGAACAAATGGCC

Another miR-29c hybrid (29c-19nt) also using the miR-155 backbone sequence is illustrated below using the same naming convention herein. Here, the top row and the bottom row represent the flanking backbone sequences of miR-155 (identical to that in the sequence immediately above), while the 2nd row is the mature miR-29c with the guide strand, loop sequence, and passenger strand. Note the loop sequence here is 19 nt, instead of the 17 nt loop in the sequence above. This 29c-19nt sequence has been incorporated into the following subject viral vector used in the examples below: EF1A-29c-19nt, 29c-19nt-μDys-pA, 29c-19nt-μDys-3UTR.

CCTGGAGGCTTGCTGAAGGCTGTATGCTG
TAGCACCATTTGAAATCGGTTA GTTTTGGCCACTGACTGAC TAACCGATCAAATGGTGCTA
CAGGACACAAGGCCTGTTACTAGCACTCACATGGAACAAATGGCC

D. Hybrid shSLN with miR-155 Backbone Sequence (shmSLN-v2 & c1/c2-m155)

A shSLN in the miR-155 backbone sequence is illustrated below using the same naming convention herein. Here, the top row and the bottom row represent the flanking backbone sequences of miR-155, while the 2nd row is the mature shRNA targeting mouse SLN (shmSLN) with the guide strand, loop sequence (19 nt), and passenger strand. This shmSLN-v2 sequence has been incorporated into the following subject viral vector used in the examples below: EF1A-mSLN, Fusion-v1, μDys-shmSLN-v1.

CCTGGAGGCTTGCTGAAGGCTGTATGCTG
GTGATGAGGACAACTGTGAAG GTTTTGGCCACTGACTGAC CTTCACAGGTCCTCATCAC
CAGGACACAAGGCCTGTTACTAGCACTCACATGGAACAAATGGCC

A shSLN in the miR-155 backbone sequence is illustrated below using the same naming convention herein. Here, the top row and the bottom row represent the flanking backbone sequences of miR-155, while the 2nd row is another mature shRNA targeting mouse SLN (shmSLN) with the guide strand, loop sequence (19 nt), and passenger strand. Compared to the similar/related shmSLN sequence above, the presence of the extra dinucleotide base pairs TT:AA (or strictly speaking, UU at the 3′ end of the siRNA) have been associated with increased potency of the produced guide strand siRNA. This shmSLN-v2 sequence has been incorporated into the following subject viral vector used in the examples below: EF1A-mSLN-v2, Fusion-v2, μDys-shmSLN-v2.

CCTGGAGGCTTGCTGAAGGCTGTATGCTG
GTGATGAGGACAACTGTGAAGTT GTTTTGGCCACTGACTGAC AACTTCACTTGTCCTCATCAC
CAGGACACAAGGCCTGTTACTAGCACTCACATGGAACAAATGGCC

Another shSLN in the miR-155 backbone sequence is illustrated below using the same naming convention herein. Here, the top row and the bottom row represent the flanking backbone sequences of miR-155, while the 2nd row is the mature shRNA targeting human SLN with the guide strand, loop sequence (19 nt), and passenger strand. This c1-m155 sequence has been incorporated into the following subject viral vector used in the examples below: c1-m155-pa, c1-m155-i2, c1-m155-3UTR.

CCTGGAGGCTTGCTGAAGGCTGTATGCTG
GTAATCAAGACAATAGTGAAGTT GTTTTGGCCACTGACTGAC
AACTTCACTTGTCTTGATTAC
CAGGACACAAGGCCTGTTACTAGCACTCACATGGAACAAATGGCC

Another shSLN in the miR-155 backbone sequence is illustrated below using the same naming convention herein. Here, the top row and the bottom row represent the flanking backbone sequences of miR-155, while the 2nd row is the mature shRNA targeting human SLN with a different guide strand, loop sequence (19 nt), and passenger strand. This c2-m155 sequence has been incorporated into the following subject viral vector used in the examples below: c2-m155-pa, c2-m155-i2, c2-m155-3UTR.

CCTGGAGGCTTGCTGAAGGCTGTATGCTG
TTGAGAAACAGCTCCCGGGTGTT GTTTTGGCCACTGACTGAC
AACACCCGGGAGCTGTTTCTCAA
CAGGACACAAGGCCTGTTACTAGCACTCACATGGAACAAATGGCC

E. Hybrid miR-29c with miR-451 Backbone Sequence (29c-451)

A miR-29c hybrid (29c-451, see FIG. 31 of PCT/US2019/065718, filed on Dec. 11, 2019 for its predicted 2D structure) using the miR-451 backbone sequence is illustrated below using the same naming convention herein. Here, the top 2 rows and the bottom 2 rows represent the flanking backbone sequences of miR-451, and the 3rd row is the mature miR-29c with the guide strand, loop sequence, and passenger strand.

GGACAGGAGAGATGCTGCAAGCCCAAGAAGCTCTCTGCTCAGCCT
GTCACAACCTACTGACTGCCAGGGCACTTGGGAATGGCAAGG
TAGCACCATTTGAAATCGGTTA CGATTTCAAATGGTGCTG
TCTTGCTATACCCAGA
AAACGTGCCAGGAAGAGAACTCAGGACCCTGAAGCAGACTACTGG
AAGGGAGACTCCAGCTCAAACAAGGCA

F. U6 Driven miR-29c and shSLN

The experimental section below also describes the use of certain “solo” viral vector constructs that express only miR-29c or only shSLN. Such solo expression cassettes are driven by the strong Pol III U6 promoter. Such sequences do not belong to modified miR-29c or modified shSLN sequences, since the strong U6 promoter directly generates the pre-miRNA or shSLN without any flanking nucleotide sequences. For comparison purpose, however, such sequences are also listed here using the same nomenclature.

A miR-29c driven by the U6 promoter is illustrated below (U6-29c-v1). Here, the 2nd row is the mature miR-29c with the passenger strand, loop sequence, and guide strand. This has been used in the pGFP-U6-shAAV-GFP vector to generate a “solo” control vector. The nucleotides in the first row of the continuous sequence below are the first 5 nucleotides after the transcription start site in the U6 promoter, and the T6 transcription termination sequence precedes the sequence used for cloning in the last row of the continuous sequence below.

GATCG
TAACCGATTTCAAATGGTGCTA GCCCTGACCCAGC
TAGCACCATTTGAAATCGGTTA TTTTTTGAAGCT

A shSLN driven by the U6 promoter is illustrated below (U6-shmSLN-v1). Here, the 2nd row is the mature shSLN with the passenger strand, loop sequence, and guide strand. This has been used in the U6-shmSLN-v1 vector in the examples.

GATCG
ACTTCACAGTTGTCCTCATCAC TCGA
GTGATGAGGACAACTGTGAAG CTTTTTTGAAGCT

A shSLN driven by the U6 promoter is illustrated below (U6-mSLN-v4). Here, the 2nd row is the mature shSLN with the passenger strand, loop sequence, and guide strand. This has been used in the U6-mSLN-v4 vector in the examples.

GATCG
ACTTCACAGTTGTCCTCATCAC TCAAGAG
GTGATGAGGACAACTGTGAAG TTTTTTGAAGCT

G. Divergent Constructs

Several miR29c coding sequences, and SLN-targeting shRNA coding sequences, as expressed from the divergent expression cassette within the subject viral vectors, are described herein below.

In general, all sequences below are directly inserted downstream of the U6 promoter transcription start site, and the TTTTTT stretch is the T6 transcription stop site. For all designs below, design strategy included engineering flanking sequences, loop and passenger strand nucleotide sequences to preserve the natural backbone 2D and 3D structures. For miRNA/shRNA designs, 2D/3D structures—defined largely by distances between stem loop and flanking nucleotide sequences, the central stem structure, the locations and sizes of the bulges, internal loops, and mismatches within the stem—are all important considerations.

### >Divergent_29c_v1

TAACCGATTTCAAATGGTGCTA GCCCTGACCCAGC
TAGCACCATTTGAAATCGGTTA TTTTTT

A miR-29c driven by the U6 promoter in the divergent expression cassette. Here, from 5′ to 3′, the mature miR-29c with the passenger strand sequence, loop sequence (double underlined), and the guide strand sequence are depicted just 5′ to the T6 transcription stop site.

### >Divergent_29c_v2

TAACCGATTTCAAATGGTGCTA TCAAGAG
TAGCACCATTTGAAATCGGTTA TTTTTT

A miR-29c driven by the U6 promoter in the divergent expression cassette. Here, from 5′ to 3′, the mature miR-29c with the passenger strand sequence, loop sequence (double underlined), and the guide strand sequence are depicted just 5′ to the T6 transcription stop site.

### >Divergent_29c_v3

TAACCGATTTCAAATGGTGCTA CTTCCTGTCAGA
TAGCACCATTTGAAATCGGTTA TTTTTT

A miR-29c driven by the U6 promoter in the divergent expression cassette. Here, from 5′ to 3′, the mature miR-29c with the passenger strand sequence, loop sequence (double underlined), and the guide strand sequence are depicted just 5′ to the T6 transcription stop site.

### >Divergent_29c_v4

TAACCGATTTCAAATGGTGCTA TTCAAGAGA
TAGCACCATTTGAAATCGGTTA TTTTTT

A miR-29c driven by the U6 promoter in the divergent expression cassette. Here, from 5′ to 3′, the mature miR-29c with the passenger strand sequence, loop sequence (double underlined), and the guide strand sequence are depicted just 5′ to the T6 transcription stop site.

>Divergent_29c_v5 (mir-155 Backbone-Based Design)

CCTGGAGGCTTGCTGAAGGCTGTATGCTG
TAGCACCATTTGAAATCGGTTA GTTTTGGCCACTGACTGAC
TAACCGATCAAATGGTGCTA
CAGGACACAAGGCCTGTTACTAGCACTCACATGGAACAAATGGCC
TTTTTT

A miR-29c driven by the U6 promoter in the divergent expression cassette. Here, the miR-29c uses the miR-155 backbone sequences, with the top row and the bottom row sequences represent the flanking backbone sequences of miR-155, while the 2nd row sequence, from 5′ to 3′, includes the mature miR-29c with the guide strand sequence, loop sequence (double underlined), and the passenger strand sequence.

### >Divergent_mSLN_shv1

ACTTCACAGTTGTCCTCATCAC TCGA GTGATGAGGACAACTGTGAAG
CTTTTTT

An shRNA targeting mSLN driven by the U6 promoter in the divergent expression cassette. Here, from 5′ to 3′, the mature shmSLN with the passenger strand sequence, loop sequence (double underlined), and the guide strand sequence are depicted just 5′ to the T6 transcription stop site.

### >Divergent_mSLN_shv2

ACTTCACAGTTGTCCTCATCAC TCGA GTGATGAGGACAACTGTGAAG
TTTTTT

An shRNA targeting mSLN driven by the U6 promoter in the divergent expression cassette. Here, from 5′ to 3′, the mature shmSLN with the passenger strand sequence, loop sequence (double underlined), and the guide strand sequence are depicted just 5′ to the T6 transcription stop site.

### >Divergent_mSLN_shv3

ACTTCACAGTTGTCCTCATCAC GCCCTGACCCAGC
GTGATGAGGACAACTGTGAAG TTTTTT

An shRNA targeting mSLN driven by the U6 promoter in the divergent expression cassette. Here, from 5′ to 3′, the mature shmSLN with the passenger strand sequence, loop sequence (double underlined), and the guide strand sequence are depicted just 5′ to the T6 transcription stop site.

### >Divergent_mSLN_shv4

ACTTCACAGTTGTCCTCATCAC TCAAGAG
GTGATGAGGACAACTGTGAAG TTTTTT

An shRNA targeting mSLN driven by the U6 promoter in the divergent expression cassette. Here, from 5′ to 3′, the mature shmSLN with the passenger strand sequence, loop sequence (double underlined), and the guide strand sequence are depicted just 5′ to the T6 transcription stop site.

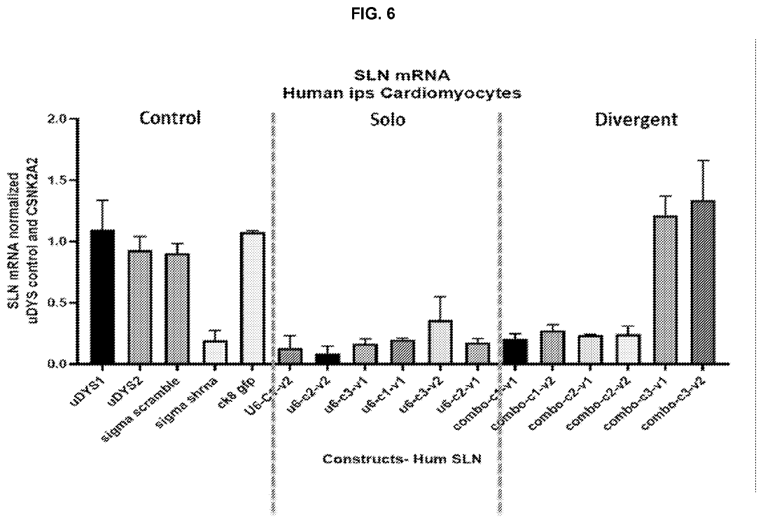

The following sequences have been used either in solo constructs encoding only shRNA targeting human SLN (shhSLN) (i.e., not in the divergent cassette), or in combination (“combo”) constructs encoding both a GOI (e.g., a μDys coding sequence) and an shRNA targeting human SLN in the divergent expression cassette. See FIG. 6.

### >U6-hSLN-c1-v1

CTTCACTATTGTCTTGATTAC TCGA GTAATCAAGACAATAGTGAAG
TTTTTT

An shRNA targeting hSLN driven by the U6 promoter. Here, from 5′ to 3′, the mature shhSLN with the passenger strand sequence, loop sequence (double underlined), and the guide strand sequence are depicted just 5′ to the T6 transcription stop site.

### >U6-hSLN-c1-v2

CTTCACTATTGTCTTGATTAC GCCCTGACCCAGC
GTAATCAAGACAATAGTGAAG TTTTTT

An shRNA targeting hSLN driven by the U6 promoter. Here, from 5′ to 3′, the mature shhSLN with the passenger strand sequence, loop sequence (double underlined), and the guide strand sequence are depicted just 5′ to the T6 transcription stop site.

### >U6-hSLN-c2-v1

CACCCGGGAGCTGTTTCTCAA TCGA TTGAGAAACAGCTCCCGGGTG
TTTTTT

An shRNA targeting hSLN driven by the U6 promoter. Here, from 5′ to 3′, the mature shhSLN with the passenger strand sequence, loop sequence (double underlined), and the guide strand sequence are depicted just 5′ to the T6 transcription stop site.

### >U6-hSLN-c2-v2

CACCCGGGAGCTGTTTCTCAA GCCCTGACCCAGC
TTGAGAAACAGCTCCCGGGTG TTTTTT

An shRNA targeting hSLN driven by the U6 promoter. Here, from 5′ to 3′, the mature shhSLN with the passenger strand sequence, loop sequence (double underlined), and the guide strand sequence are depicted just 5′ to the T6 transcription stop site.

### >U6-hSLN-c3-v1

TGTGGCTCCTTGTGAGGTCCT TCGA AGGACCTCACAAGGAGCCACA
TTTTTT

An shRNA targeting hSLN driven by the U6 promoter. Here, from 5′ to 3′, the mature shhSLN with the passenger strand sequence, loop sequence (double underlined), and the guide strand sequence are depicted just 5′ to the T6 transcription stop site.

### >U6-hSLN-c3-v2

TGTGGCTCCTTGTGAGGTCCT GCCCTGACCCAGC
AGGACCTCACAAGGAGCCACA TTTTTT

An shRNA targeting hSLN driven by the U6 promoter. Here, from 5′ to 3′, the mature shhSLN with the passenger strand sequence, loop sequence (double underlined), and the guide strand sequence are depicted just 5′ to the T6 transcription stop site.

The viral vector of the invention can be used in gene therapy to treat a number of genetic disorders, including but are not limited to the various muscular dystrophy as described above. Some of the additional genetic disorders that can be treated using the divergent vectors of the invention are described in further sections below.

### Alpha-1 Antitrypsin Deficiency (A1AD or AATD) Treatment

Alpha-1 antitrypsin deficiency (A1AD or AATD) is a genetic disorder due to a mutation in the SERPINA1 (Serpin peptidase inhibitor, clade A, member 1) gene that results in insufficient A1AT protein (a serpin superfamily protease inhibitor) being produced. Alpha-1 antitrypsin deficiency occurs worldwide, but its prevalence varies by population. This disorder affects about 1 in 1,500 to 3,500 individuals with European ancestry. It is uncommon in people of Asian descent. Despite its name, A1AT, being a protease inhibitor, does not inhibit just trypsin. It predominantly binds/complexes with elastases, but also with trypsins, chymotrypsins, thrombins, and bacterial proteases. It is produced in the liver, and normally joins systemic circulation. Its reference range in the blood is between 0.9-2.3 g/L, but its concentration can rise many folds upon acute inflammation.

A1AT protects tissues from enzymes of inflammatory cells, especially neutrophil elastase. When the blood contains inadequate amounts of A1AT or functionally defective A1AT (such as in alpha-1 antitrypsin deficiency), neutrophil elastase is excessively free to break down elastin, degrading the elasticity of the lungs, which results in respiratory complications, such as chronic obstructive pulmonary disease (COPD) in adults.

Further, defective A1AT can fail to leave liver, its site of origin, and instead build up in the liver, resulting in cirrhosis in either adults or children. For example, the so-called Z mutation/allele causes the A1AT protein to polymerize in hepatocytes, preventing its secretion into the blood, and the aggregated mutant proteins have toxic gain of function (on the other hand, animal data has shown that a mere reduction in the level of the mutated protein has beneficial effect, even in the absence of wild-type protein up-regulation).

Thus, the disease A1AD may manifest as lung disease and/or liver disease, and the underlying mechanisms may involve unblocked neutrophil elastase and buildup of abnormal A1AT in the liver. It is autosomal co-dominant, in that one defective allele tends to result in milder disease than two defective alleles. Symptoms of the lung disease include shortness of breath, wheezing, or an increased risk of lung infections. Complications of A1AD may also include COPD, cirrhosis, neonatal jaundice, or panniculitis.

A1AT is a single-chain glycoprotein consisting of 394 amino acids in the mature form, and exhibits many glycoforms. The mature A1AT protein can be encoded by a polynucleotide of about 1.2 kb (1254 nts) of the SERPINA1 gene, which has been localized to human chromosome 14q32. Over 75 mutations of the SERPINA1 gene have been identified, many with clinically significant effects (see Silverman et al., Alpha1-Antitrypsin Deficiency. New England Journal of Medicine 360 (26): 2749-2757, 2009 (incorporated herein by reference).

For example, the most common cause of a severe deficiency, PiZ (i.e., the Z allele), is a single base-pair substitution leading to a glutamate to lysine mutation at position 342 (E342K, or Glu342Lys, see table below). Homozygous ZZ phenotype is associated with high risk of emphysema and liver disease. Meanwhile, PiS (i.e., the S allele) is caused by a glutamate to valine mutation at position 264 (E264V, or Glu264Val, see table below). SS homozygous is at no risk of emphysema, but S and Z or S and null heterozygotes have a mildly increased risk of emphysema. Other rarer forms, such as the PiM (Malton) allele associated with increased risk for both liver and lung diseases, have also been described, and some of which are listed in the table below with the various nomenclature used in the art. Other alleles include the Pittsburgh allele (Met358Arg), which occurs at the A1AT active site and alters its function to become a potent inhibitor for thrombin and factor XI rather than elastase, resulting in bleeding disorder.

At present, treatment of the lung disease includes bronchodilators, inhaled steroids, and when infections occur, antibiotics. Intravenous infusions of the A1AT protein, or in severe disease, lung transplantation may also be recommended. In those with severe liver disease, liver transplantation may be an option. Vaccination for influenza, pneumococcus, and hepatitis is also recommended.

Therefore, the AAV vector of the invention can be used to treat A1AD, in that the divergent or fusion vector of the invention can be used to deliver (1) a first therapeutic agent comprising a coding sequence for wild-type SERPINA1, and (2) a second therapeutic agent comprising an antagonist for a mutant SERPINA1, such that expression of the defective A1AT protein is reduced or eliminated.

For example, the wt SERPINA1 gene can be a 1257 nts polynucleotide sequence (see below), and can be expressed in liver using any one of liver specific enhancers and/or promoters.

ATGCCGTCTTCTGTCTCGTGGGGCATCCTCCTGCTGGCAGGCCTGTGCTG
CCTGGTCCCTGTCTCCCTGGCTGAGGATCCCCAGGGAGATGCTGCCCAGA
AGACAGATACATCCCACCATGATCAGGATCACCCAACCTTCAACAAGATC
ACCCCCAACCTGGCTGAGTTCGCCTTCAGCCTATACCGCCAGCTGGCACA
CCAGTCCAACAGCACCAATATCTTCTTCTCCCCAGTGAGCATCGCTACAG
CCTTTGCAATGCTCTCCCTGGGGACCAAGGCTGACACTCACGATGAAATC
CTGGAGGGCCTGAATTTCAACCTCACGGAGATTCCGGAGGCTCAGATCCA
TGAAGGCTTCCAGGAACTCCTCCGTACCCTCAACCAGCCAGACAGCCAGC
TCCAGCTGACCACCGGCAATGGCCTGTTCCTCAGCGAGGGCCTGAAGCTA
GTGGATAAGTTTTTGGAGGATGTTAAAAAGTTGTACCACTCAGAAGCCTT
CACTGTCAACTTCGGGGACACCGAAGAGGCCAAGAAACAGATCAACGATT
ACGTGGAGAAGGGTACTCAAGGGAAAATTGTGGATTTGGTCAAGGAGCTT
GACAGAGACACAGTTTTTGCTCTGGTGAATTACATCTTCTTTAAAGGCAA
ATGGGAGAGACCCTTTGAAGTCAAGGACACCGAGGAAGAGGACTTCCACG
TGGACCAGGTGACCACCGTGAAGGTGCCTATGATGAAGGGTTTAGGCATG
TTTAACATCCAGCACTGTAAGAAGCTGTCCAGCTGGGTGCTGCTGATGAA
ATACCTGGGCAATGCCACCGCCATCTTCTTCCTGCCTGATGAGGGGAAAC
TACAGCACCTGGAAAATGAACTCACCCACGATATCATCACCAAGTTCCTG
GAAAATGAAGACAGAAGGTCTGCCAGCTTACATTTACCCAAACTGTCCAT
TACTGGAACCTATGATCTGAAGAGCGTCCTGGGTCAACTGGGCATCACTA
AGGTCTTCAGCAATGGGGGTGACCTCTCCGGGGTCACAGAGGAGGCACCC
CTGAAGCTCTCCAAGGCCGTGCATAAGGCTGTGCTGACCATCGACGAGAA
AGGGACTGAAGCTGCTGGGGCCATGTTTTTAGAGGCCATACCCATGTCTA
TCCCCCCCGAGGTCAAGTTCAACAAACCCTTTGTCTTCTTAATGATTGAA
CAAAATACCAAGTCTCCCCTCTTCATGGGAAAAGTGGTGAATCCCACCCA
AAAATAA

A liver promoter (e.g. liver-specific ApoE enhancer and al-antitrypsin promoter can be found at www.ncbi.nlm.nih.gov/pubmed/8845389, which drives expression of M-form SERPINA1, and any RNAi agent against SERPINA1 variant forms that cause AATD.

A codon optimized SERPINA1 for human expression (see below) can also be used.

ATGCCAAGTAGTGTATCCTGGGGAATTCTGCTCTTGGCTGGGCTCTGTTG
CCTTGTCCCAGTGTCTCTTGCCGAAGACCCTCAGGGTGACGCAGCTCAGA
AAACCGATACCAGTCATCACGATCAAGATCACCCTACTTTCAATAAAATA
ACGCCCAACCTTGCAGAATTTGCGTTCTCTCTGTATCGGCAGCTCGCGCA
CCAGTCCAATTCAACCAACATATTTTTCTCACCGGTTAGCATCGCAACTG
CGTTCGCAATGTTGTCCCTCGGTACAAAAGCCGACACGCATGACGAAATT
TTGGAAGGACTGAACTTTAATCTGACCGAGATACCGGAGGCCCAGATTCA
CGAGGGATTTCAAGAGCTTCTTCGCACACTCAACCAACCGGATTCTCAGC
TGCAGTTGACAACTGGGAACGGTCTCTTTCTGTCTGAGGGACTTAAACTT
GTAGACAAATTTCTTGAGGATGTCAAGAAGCTCTACCACTCCGAAGCTTT
TACGGTTAATTTCGGGGATACGGAAGAGGCGAAAAAGCAAATAAACGATT
ATGTGGAAAAAGGAACACAGGGTAAAATCGTTGATTTGGTAAAGGAGCTG
GACAGGGACACAGTTTTCGCTCTGGTAAATTACATATTCTTTAAGGGCAA
ATGGGAGCGACCGTTCGAAGTCAAAGACACCGAGGAAGAGGACTTTCACG
TGGACCAAGTGACCACCGTAAAGGTCCCTATGATGAAACGCCTCGGGATG
TTTAATATCCAACACTGTAAAAAATTGTCCAGCTGGGTCTTGCTGATGAA
GTATTTGGGAAATGCAACCGCAATTTTTTTTCTGCCTGATGAAGGGAAGC
TTCAGCATTTGGAAAACGAGCTGACACATGATATAATCACCAAATTTCTG
GAAAATGAAGACAGAAGGTCCGCTAGTTTGCATTTGCCTAAATTGTCCAT
AACAGGCACATACGATCTTAAGTCCGTCCTTGGACAACTCGGAATCACAA
AGGTCTTCAGTAACGGTGCCGATCTGAGTGGAGTCACAGAAGAAGCCCCT
TTGAAATTGTCCAAAGCGGTACATAAAGCAGTGCTTACTATAGACGAAAA
GGGCACTGAAGCAGCAGGGGCGATGTTCTTGGAGGCGATACCAATGTCCA
TTCCACCAGAGGTCAAGTTCAACAAGCCCTTCGTATTTCTCATGATCGAA
CAGAATACAAAAAGTCCCCTCTTTATGGGTAAGGTCGTTAATCCCACCCA
AAAGTAA

In certain embodiments, the antagonist encodes an RNAi reagent (such as siRNA, miRNA, shRNA etc) that targets the mRNA of the defective A1AT (but not the wild-type A1AT).

In certain embodiments, the siRNA targeting locations can include the 3′UTR, 5′UTR, and/or coding regions of SERPINA1, since SERPINA1 gene to be expressed from the subject viral vectors can have codon optimized coding sequences and optimized UTRs that will be different from the natural SERPINA1 gene sequence, and are thus not targeted by the siRNA against the mutant SERPINA1. A few representative shRNA sequences targeting mutant SERPINA1 are provided below for illustration purpose.

>SERPINA1-siRNA-1 (the second line represents the passenger strand,
the loop, and the guide strand sequences):
GATCG
CTGTGTTCATGGAGCATCTGG TCAAGAG CCAGATGCTCCATGAACACAG
TTTTTTGAAGCT
>SERPINA1-siRNA-2 (the second line represents the passenger strand,
the loop, and the guide strand sequences)
GATCG
CTGTGTTCATGGAGCATCTGG GCCCTGACCCAGC CCAGATGCTCCATGAACACAG
TTTTTTGAAGCT

In certain embodiments, the defective A1AT contains the B (Alhambra) allele, the M (Malton) allele, the S allele, the M (Heerlen) allele, the M (Mineral Springs) allele, the M (procida) allele, the M (Nichinan) allele, the I allele, the P (Lowell) allele, the null (Granite falls) allele, the null (Bellingham) allele, the null (Mattawa) allele, the null (procida) allele, the null (Hong Kong 1) allele, the null (Bolton) allele, the Pittsburgh allele, the V (Munich) allele, the Z (Augsburg) allele, the W (Bethesda) allele, the null (Devon) allele, the null (Ludwigshafen) allele, the Z (Wrexham) allele, the null (Hong Kong 2) allele, the null (Riedenburg) allele, the Kalsheker-Poller allele, the P (Duarte) allele, the null (West) allele, the S (Iiyama) allele, and the Z (Bristol) allele.

In certain embodiments, the defective A1AT contains the Pittsburgh allele, and/or one or more of the mutations listed in the table below.

In case where two mutant alleles are present, the second therapeutic agent may encode multiple antagonists, each targeting a specific region of at least one of the mutant alleles (but spares the wild-type allele encoded by the same vector). For example, one antagonist may be an shRNA or siRNA or miRNA targeting the Z allele mutation, and another antagonist may be an shRNA or siRNA or miRNA targeting the S allele mutation, etc.

In certain embodiments, the subject vector may encode multiple antagonists against multiple defective A1AT alleles, with or without encoding a wild-type allele.

As used herein, “wild-type allele” refers to a variant that has normal (not defective) level of A1AT inhibitory activity, including the M1A (Ala at residue 213), M1V (Val at residue 213), M2, M3 alleles, etc. (Crystal, Trends Genetics 5:411-7, 1989, incorporated by reference).

| Nucleotide | Protein nomenclature | Protein nomenclature | |
|---|---|---|---|
| nomenclature^{a} | (Unprocessed) | (legacy) | Synonyms |
| 17 kb deletion of all coding exons | | | Nullisola_{di procida}; Null_{procida} |
| c.17C > T | p.Ser6Leu | p.Ser-19Leu | Z_{wrexham} |
| c.194T > C | p.Leu65Pro | p.Leu41Pro | M_{procida} |
| c.227_229delTCT | p.Phe76del | p.Phe52del | Mₘₐₗₜₒₙ; Mₚₐₗₑᵣₘₒ |
| c.230C > T | p.Ser77Phe | p.Ser53Phe | S_{iiyama} |
| c.272G > A | p.Gly91Glu | p.Gly67Glu | M_{mineral springs} |
| c.275C > T | p.Thr92Ile | p.Thr68Ile | Null_{lisbon} |
| c.347T > A | p.Ile116Asn | p.Ile92Asn | Null_{ludwigshafen} |
| c.415G > A | p.Gly139Ser | p.Gly115Ser | Null_{devon}; Null_{newport} |
| c.552delC | p.Tyr184X | p.Tyr160Ter | Null_{granite falls} |
| c.646 + 1G > T | | | Null_{west} |
| c.721A > T | p.Lys241X | p.Lys217Ter | Null_{bellingham} |
| c.739C > T | p.Arg247Cys | p.Arg223Cys | F |
| c.839A > T | p.Asp280Val | p.Asp256Val | P_{duarte}; P_{lowell}; Null_{cardiff} |
| c.863A > T^{b} | p.Glu288Val | p.Glu264Val | S |
| c.1027_1028delTC | p.Ser343ArgfsX16 | p.Ser319ArgfsX16 | Null_{hong kong 1} |
| c.1078G > A | p.Ala360Thr | p.Ala336Thr | W_{bethesda} |
| c.1096G > A^{b} | p.Glu366Lys | p.Glu342Lys | Z |
| 1130dupT | p.Leu377PhefsX24 | p.Leu353PhefsX24 | Null_{mattawa} |
| c.1158delC | p.Glu387ArgfsX11 | p.Glu363ArgfsX11 | Null_{bolton} |
| c.1158dupC | p.Glu387ArgfsX14 | p.Glu363ArgfsX14 | Null_{saarbruecken} |
| c.1178C > T | p.Pro393Leu | p.Pro369Leu | Mₕₑₑᵣₗₑₙ |

In certain embodiments, the viral vector of the invention preferentially infect liver cells and tissues. For example, pseudo-serotyping a recombinant AAV2 vector genome with the AAV8 capsid (designated AAV2/8) enhances tropism for hepatocytes.

### Repeat Expansion Disorders

The vectors of the invention can also be used to treat certain so-called Repeat Expansion Disorders (REDs) caused by expanded nucleotide repetitions that can occur throughout a gene.

Examples of such REDs include triplet nucleotide repeats in the 5′UTR region (such as Fragile X Symdrom (CGG repeat), FXTAS (CGG repeat), Fragile XE mental retardation (GCC repeat), and Spinocerebella ataxia type 12 (CAG repeat)); triplet nucleotide repeats in the exons (such as Spinocerebella ataxia type 1, 2, 3, 6, 7, and 17 (CAG repeat), Huntington's Disease (CAG repeat), Huntington's Disease-Like 2 (CAG repeat), Spino-bulbar muscular atrophy (CAG repeat), Dentatorubral-pallidoluysian atrophy (CAG repeat), multiple skeletal ysplasias (GAC repeat), Synpolydactyly syndrome (GCG repeat), hand-foot-genital syndrome (GCG repeat), Cleidocranial dysplasia (GCG repeat), holoprosencephaly (GCG repeat), oculopharyngeal muscular dystrophy (GCG repeat), congenital central hypoventilation syndrome (GCG repeat), BPEI syndrome (GCG repeat), and X-linked mental retardation (GCG repeat)); nucleotide repeats in the introns (such as Friedreich's ataxia (GAA repeat), myotonic dystrophy type 2 (CCTG repeat), Spinocerebella ataxia type 10 (ATTCT repeat), Spinocerebella ataxia type 31 (TGGAA repeat), Spinocerebella ataxia type 36 (GGCCTG repeat), and amyotrophic lateral sclerosis (GGGGCC repeat)); and triplet nucleotide repeats in the 3′UTR region (such as myotonic dystrophy type 1 (CTG repeat) and Spinocerebella ataxia type 8 (CTG repeat)).

For example, spinocerebellar ataxia (SCA) is a group of hereditary ataxias that are characterized by degenerative changes in the part of the brain related to the movement control (cerebellum), and sometimes in the spinal cord. There are numerous types of SCA, according to their order of identification (see SCA1-45 in the OMIM website), classified according to the mutated (altered) gene responsible for the specific type of SCA. The signs and symptoms may vary by type but are similar, and may include an uncoordinated walk (gait), poor hand-eye coordination, and abnormal speech (dysarthria). SCA3, also known as Machado-Joseph disease, is the most common type of SCA. SCA types 9 through 36 are rare and less well characterized.

Selected SCA, including their loci on the human chromosome, gene product including the size of the encoded proteins, as well as the type of underlying mutations, are summarized in the table below.

| Disease | Locus | Gene (product) | Type of Mutation |
|---|---|---|---|
| SCA1 | 6p22.3 | SCA1 (ataxin-1) | CAG/polyQ expansion |
| | | 815aa | |
| SCA2 | 12q24.12 | SCA2 (ataxin-2) | CAG/polyQ expansion |
| | | 1153aa | |
| SCA3 | 14q32.21 | MJD1 (ataxin-3 or | CAG/polyQ expansion |
| (MJD) | | MJDp) 346aa | |
| SCA4 | 16q22.1 | Unknown | Unknown |
| SCA5 | 11p11-q11 | β III Spectrin | Nonrepeat mutations |
| SCA6 | 19p13.2 | CACNA1 ^{~}1600aa | CAG/polyQ expansion |
| SCA7 | 3p14.1 | Ataxin-7 892aa | CAG/polyQ expansion |
| SCA8 | 13q21.33 | SCA8 | CTG/CAG expansion |
| SCA10 | 22q13 | SCA10 | ATTCT expansion |
| SCA11 | 15q14-21.3 | Unknown | Unknown |
| SCA12 | 5q32 | PPP2R2B | CAG expansion |
| SCA13 | 19q13.3-13.4 | KCNC3 | Nonrepeat mutations |
| SCA14 | 19q13.4-q ter | PKRCG | Nonrepeat mutations |
| SCA15 | 3pter-q24.2 | Unknown | Unknown |
| SCA16 | 8q23-24.1 | Unknown | Unknown |
| SCA17 | 6q27 | TBP | CAG/polyQ expansion |
| SCA18 | 7q31-32 | Unknown | Unknown |
| SCA19 | 1p21-q21 | Unknown | Unknown |
| SCA21 | 7p21.3-15.1 | Unknown | Unknown |
| SCA22 | 1p210q23 | Unknown | Unknown |
| SCA23 | 20p13-12.2 | Unknown | Unknown |
| SCA24 | 1p36 | Unknown | Unknown |
| SCA25 | 2p21-p15 | Unknown | Unknown |
| SCA26 | 19p13.3 | Unknown | Unknown |
| SCA27 | 13 | FGF14 | Nonrepeat mutations |
| SCA28 | 18p11.22-q11.2 | Unknown | Unknown |

SCA is inherited in an autosomal dominant manner. Other diseases using the term spinocerebellar may have an autosomal recessive inheritance (“SCAR”). Current Treatment is supportive and is based on the signs and symptoms (rather than the cause) present in the person with SCA.

One common feature of these REDs is that they all involve nucleotide repeats, predominantly trinucleotide repeat, which is a segment of DNA that is repeated numerous times. While it may not be abnormal for these repeats to exist and cause no problems, a greater than normal number of repeats can interfere with the function of the affected gene, resulting in a genetic condition. Such trinucleotide repeats are unstable and can change in length when passed from parent to child. An increased number of repeats often leads to an earlier age of onset and more severe disease.

In autosomal dominant conditions, one mutated copy of the responsible gene in each cell is enough to cause signs or symptoms of the condition. Thus, successful treatment of such RED may require both replacing the defective gene, but also reduce or eliminate the defective gene or gene product.

As a specific example, the most common SCA is SCA3, or Machado-Joseph disease (MJD), which is is an autosomal dominant progressive neurologic disorder characterized principally by ataxia, spasticity, and ocular movement abnormalities. It is estimated that MJD affects 1-5 per 100,000 worldwide. MJD is caused by a heterozygous (CAG)ₙ trinucleotide repeat expansion encoding Gln repeats in the ataxin-3 gene (ATXN3) on chromosome 14q32. Normal individuals have up to 44 Q repeats, and MJD patients have between 52 and 86 Q repeats. Alves et al. (PLOS ONE 3(10): e3341, doi.org/10.1371/journal.pone.0003341) showed that a single nucleotide polymorphism (SNP) is present in more than 70% of patients with Machado-Joseph disease (MJD), and this SNP can be used to inactivate the mutant ataxin-3 allele selectively, using a Lentiviral vector-mediated RNAi (shRNA) both in vitro and in a rat model of MJD in vivo. The selectivity of the approach was demonstrated in vitro, by the preservation of wild-type ataxin-3 expression upon co-expression of the mutant allele-specific shRNA, and in vivo, by the limited effect of wild-type allele-specific shRNA on mutant ataxin-3 gene expression. The allele-specific silencing of ataxin-3 significantly decreased the severity of the neuropathological abnormalities associated with MJD. Thus a similar approach can be used when the vector of the invention can be used to simultaneously deliver an antagonist of the defective SCA3 allele, and a wild-type allele of SCA3 that cannot be targeted by the mutant allele-specific antagonist.

The same approach can be used for any other REDs, when there is SNP between the wild-type allele and the mutant allele.

Therefore, the AAV vector of the invention can be used to treat any of the REDs, such as those described herein, in that the divergent or fusion vector of the invention can be used to deliver (1) a first therapeutic agent comprising a coding sequence for a wild-type gene underlying the RED to be treated, and (2) a second therapeutic agent comprising an antagonist for a mutant RED gene, such that expression of the defective RED gene and/or protein is reduced or eliminated.

In certain embodiments, the RED is SCA1, 2, 3, 6, 7, 8, 10, 12, or 17, respectively, wherein the first therapeutic agent comprises a coding sequence for wild-type ataxin-1, ataxin-2, ataxin-3, CACNA1, ataxin-7, SCA8, SCA10, PPP2R2B, or TBP, respectively, and (2) the second therapeutic agent comprises an antagonist specific for the mutant allele of ataxin-1, ataxin-2, ataxin-3, CACNA1, ataxin-7, SCA8, SCA10, PPP2R2B, or TBP, respectively.

For example, the wt ataxin-3 gene ATXN3 can be encoded by a 1086 nts polynucleotide sequence (see below), and can be expressed in neurons using any one of neuron specific promoters such as the synapsin promoter.

ATGGAGTCCATCTTCCACGAGAAACAAGAAGGCTCACTTTGTGCTCAACATTGCCTGAATAACTTATT
GCAAGGAGAATATTTTAGCCCTGTGGAATTATCCTCAATTGCACATCAGCTGGATGAGGAGGAGAGGA
TGAGAATGGCAGAAGGAGGAGTTACTAGTGAAGATTATCGCACGTTTTTACAGCAGCCTTCTGGAAAT
ATGGATGACAGTGGTTTTTTCTCTATTCAGGTTATAAGCAATGCCTTGAAAGTTTGGGGTTTAGAACT
AATCCTGTTCAACAGTCCAGAGTATCAGAGGCTCAGGATCGATCCTATAAATGAAAGATCATTTATAT
GCAATTATAAGGAACACTGGTTTACAGTTAGAAAATTAGGAAAACAGTGGTTTAACTTGAATTCTCTC
TTGACGGGTCCAGAATTAATATCAGATACATATCTTGCACTTTTCTTGGCTCAATTACAACAGGAAGG
TTATTCTATATTTGTCGTTAAGGGTGATCTGCCAGATTGCGAAGCTGACCAACTCCTGCAGATGATTA
GGGTCCAACAGATGCATCGACCAAAACTTATTGGAGAAGAATTAGCACAACTAAAAGAGCAAAGAGTC
CATAAAACAGACCTGGAACGAGTGTTAGAAGCAAATGATGGCTCAGGAATGTTAGACGAAGATGAGGA
GGATTTGCAGAGGGCTCTGGCACTAAGTCGCCAAGAAATTGACATGGAAGATGAGGAAGCAGATCTCC
GCAGGGCTATTCAGCTAAGTATGCAAGGTAGTTCCAGAAACATATCTCAAGATATGACACAGACATCA
GGTACAAATCTTACTTCAGAAGAGCTTCGGAAGAGACGAGAAGCCTACTTTGAAAAACAGCAGCAAAA
GCAGCAACAGCAGCAGCAGCAGCAGCAGCAGGGGGACCTATCAGGACAGAGTTCACATCCATGTGAAA
GGCCAGCCACCAGTTCAGGAGCACTTGGGAGTGATCTAGGTGATGCTATGAGTGAAGAAGACATGCTT
CAGGCAGCTGTGACCATGTCTTTAGAAACTGTCAGAAATGATTTGAAAACAGAAGGAAAAAAATAA

A codon optimized ATXN3 for human expression (see below) can also be used.

ATGGAAAGTATCTTTCATGAAAAGCAAGAGGGAAGTTTGTGCGCTCAACATTGTCTTAACAACTTGCT
GCAAGGGGAGTATTTCAGTCCTGTGGAGCTCTCCAGCATAGCACACCAACTCGATGAAGAAGAGAGAA
TGCGGATGGCCGAGGGTGGTGTTACTTCAGAGGATTACCGCACTTTCTTGCAGCAGCCCAGCGGGAAC
ATGGACGACTCTGGTTTCTTCTCTATCCAGGTCATAAGTAACGCGCTCAAGGTCTGGGGTTTGGAACT
CATTCTCTTCAACTCCCCTGAGTATCAGAGACTGAGAATAGACCCTATAAACGAGAGATCCTTTATAT
GTAATTACAAAGAACATTGGTTTACTGTACGGAAATTGGGCAAGCAGTGGTTCAATCTGAACTCATTG
CTGACGGGTCCCGAGCTCATATCCGACACGTATTTGGCGCTTTTTCTCGCCCAGTTGCAGCAGGAAGG
TTATTCCATTTTTGTAGTAAAGGGAGACCTTCCGGATTGTGAGGCGGATCAGCTTTTGCAGATGATCC
GGGTTCAGCAAATGCATAGACCGAAGTTGATTGGCGAGGAACTTGCTCAACTCAAAGAGCAGCGCGTA
CACAAAACAGACTTGGAGCGGGTTCTCGAAGCAAATGATGGAAGCGGAATGCTGGACGAAGACGAGGA
AGATCTGCAACGAGCCCTCGCTTTGAGTCGACAAGAGATAGACATGGAAGATGAAGAGGCTGACTTGA
GACGAGCGATACAGCTGTCCATGCAGGGAAGTTCCCGGAACATATCTCAAGACATGACGCAAACGAGT
GGCACAAATCTCACTAGTGAAGAGCTTAGAAAGAGACGCGAAGCTTACTTCGAGAAGCAACAGCAGAA
ACAACAACAACAGCAGCAACAGCAACAACAGGGTGATCTCAGCGGGCAAAGTAGCCACCCGTGCGAGA
GGCCCGCAACCAGCAGTGGGGCGCTGGGGTCAGATCTGGGAGATGCAATGTCAGAAGAAGATATGTTG
CAGGCCGCCGTTACTATGTCACTCGAAACAGTCCGCAATGATCTGAAAACAGAAGGTAAAAAGTAA

In certain embodiments, the antagonist encodes an RNAi reagent (such as siRNA, miRNA, shRNA etc) that targets the mRNA of the defective ataxin-1, ataxin-2, ataxin-3, CACNA1, ataxin-7, SCA8, SCA10, PPP2R2B, or TBP (but not their wild-type counterpart). The allele specificity may be based on SNP.

In certain embodiments, the siRNA targeting locations can include the CAG repeat, 3′UTR, 5′UTR, and/or coding regions of ATXN3, since ATXN3 gene to be expressed from the subject viral vectors can have codon optimized coding sequences and optimized UTRs that will be different from the natural ATXN3 gene sequence, and are thus not targeted by the siRNA against the mutant ATXN3. A few representative shRNA sequences targeting mutant ATXN3 are provided below for illustration purpose.

>ATXN3-siRNA-1 (the Second Line Represents the Passenger Strand, the Loop, and the Guide Strand Sequences):

GATCG
TGCGTCGGTTGTAGGACTAAA TCAAGAG TTTAGTCCTACAACCGACGCA
TTTTTTGAAGCT

>ATXN3-siRNA-2 (the Second Line Represents the Passenger Strand, the Loop, and the Guide Strand Sequences):

GATCG
TGCGTCGGTTGTAGGACTAAA GCCCTGACCCAGC
TTTAGTCCTACAACCGACGCA TTTTTTGAAGCT

>ATXN3-siRNA-3 (Mir-30 Backbone) (the Second Line Represents the Passenger Strand, the Loop, and the Guide Strand Sequences):

AACAGAAGGCTCGAGAAGGTATATTGCTGTTGACAGTGAGCG
TGCGTCGGTTGTAGGACTAAA TAGTGAAGCCACAGATGTA
TTTAGTCCTACAACCGACGCA
TGCCTACTGCCTCGGACTTCAAGGGGCTAGAATTCGA

>ATXN3-siRNA-4 (Mir-30 Backbone) (the Second Line Represents the Passenger Strand, the Loop, and the Guide Strand Sequences):

AACAGAAGGCTCGAGAAGGTATATTGCTGTTGACAGTGAGCG
AATGCGTCGGTTGTAGGACTA TAGTGAAGCCACAGATGTA
TAGTCCTACAACCGACGCATT
TGCCTACTGCCTCGGACTTCAAGGGGCTAGAATTCGA

The ATXN3 and/or the encoded RNAi agent (such as shRNA) can be expressed from any neuron selective promoters, such as the synapsin promoter (www.ncbi.nlm.nih.gov/pmc/articles/PMC4229583/), or a natural ATXN3 promoter, or a U6 promoter (especially for shRNA).

Another RED treatable by the vector of the invention is myotonic dystrophy type 1 (DM1), which is an autosomal dominant multisystem genetic disorder that affects skeletal and smooth muscle, as well as the eye, heart, endocrine system, and central nervous system. The clinical findings, which span a continuum from mild to severe, have been categorized into three somewhat overlapping phenotypes: mild, classic, and congenital. Currently, there is no cure for DM1. Treatment is based on the signs and symptoms present.

In contrast to myotonic dystrophy type 2 or DM2 having CCTG nucleotide expansion in intron 1 of the ZNF9 gene, which is rare and is associated with milder symptoms, DM1 is caused by expansion of a CTG trinucleotide repeat in the non-coding 3′-UTR region of the DMPK gene. CTG repeat length between 5-34 is considered normal, with 35-49 repeats considered mutable normal, while repeats exceeding 50 are considered full-penetrance abnormal. Molecular genetic testing detects pathogenic variants in nearly 100% of affected individuals. DM1 is estimated to affect 1 in 8,000 worldwide.

The protein made by the DMPK gene—myotonic dystrophy protein kinase—is believed to play a role in communication and impulse transmission within and between cells. It appears to be important for the correct functioning of cells in the heart, brain, and skeletal muscles. The more than normal number of CTG repeats leads to the creation of longer and toxic RNA. This in turn causes problems for cells, mainly because it traps and disables important proteins. This prevents cells in muscles and other tissues from functioning normally, leading to the signs and symptoms of MD1. Thus, an important strategy in designing DM1 treatment is to free cellular proteins—particularly one called muscleblind 1 or MBNL1—from their RNA web, and/or to increase the expression of MBNL1.

Thus in certain embodiments, the RED is DM1, wherein the first therapeutic agent comprises a coding sequence for wild-type DMPK (e.g., having normal number of 5-34 CTG repeats, preferably having 12 or less CTG repeats—the average number of CTG repeats found in normal or non-DM1 cells), and (2) the second therapeutic agent comprises an antagonist specific for the mutant allele of DMPK (such as those with more than 50 CTG repeats).

For example, the wt DMPK gene can be encoded by 1887 nts (see below for one of the few DMPK isoforms available), and can be expressed either ubiquitously, or specifically in muscle using any one of muscle specific promoters such as CK8.

ATGGGAGGGCATTTTTGGCCCCCAGAACCTTACACGGTGTTTATGTGGGGAAGCCCCTGGGAAGCAGA
CAGTCCTAGGGTGAAGCTGAGAGGCAGAGAGAAGGGGAGACAGACAGAGGGTGGGGCTTTCCCCCTTG
TCTCCAGTGCCCTTTCTGGTGACCCTCGGTTCTTTTCCCCCACCACCCCCCCAGCGGAGCCCATCGTG
GTGAGGCTTAAGGAGGTCCGACTGCAGAGGGACGACTTCGAGATTCTGAAGGTGATCGGACGCGGGGC
GTTCAGCGAGGTAGCGGTAGTGAAGATGAAGCAGACGGGCCAGGTGTATGCCATGAAGATCATGAACA
AGTGGGACATGCTGAAGAGGGGCGAGGTGTCGTGCTTCCGTGAGGAGAGGGACGTGTTGGTGAATGGG
GACCGGCGGTGGATCACGCAGCTGCACTTCGCCTTCCAGGATGAGAACTACCTGTACCTGGTCATGGA
GTATTACGTGGGCGGGGACCTGCTGACACTGCTGAGCAAGTTTGGGGAGCGGATTCCGGCCGAGATGG
CGCGCTTCTACCTGGCGGAGATTGTCATGGCCATAGACTCGGTGCACCGGCTTGGCTACGTGCACAGG
GACATCAAACCCGACAACATCCTGCTGGACCGCTGTGGCCACATCCGCCTGGCCGACTTCGGCTCTTG
CCTCAAGCTGCGGGCAGATGGAACGGTGCGGTCGCTGGTGGCTGTGGGCACCCCAGACTACCTGTCCC
CCGAGATCCTGCAGGCTGTGGGCGGTGGGCCTGGGACAGGCAGCTACGGGCCCGAGTGTGACTGGTGG
GCGCTGGGTGTATTCGCCTATGAAATGTTCTATGGGCAGACGCCCTTCTACGCGGATTCCACGGCGGA
GACCTATGGCAAGATCGTCCACTACAAGGAGCACCTCTCTCTGCCGCTGGTGGACGAAGGGGTCCCTG
AGGAGGCTCGAGACTTCATTCAGCGGTTGCTGTGTCCCCCGGAGACACGGCTGGGCCGGGGTGGAGCA
GGCGACTTCCGGACACATCCCTTCTTCTTTGGCCTCGACTGGGATGGTCTCCGGGACAGCGTGCCCCC
CTTTACACCGGATTTCGAAGGTGCCACCGACACATGCAACTTCGACTTGGTGGAGGACGGGCTCACTG
CCATGGAGACACTGTCGGACATTCGGGAAGGTGCGCCGCTAGGGGTCCACCTGCCTTTTGTGGGCTAC
TCCTACTCCTGCATGGCCCTCAGGGACAGTGAGGTCCCAGGCCCCACACCCATGGAACTGGAGGCCGA
GCAGCTGCTTGAGCCACACGTGCAAGCGCCCAGCCTGGAGCCCTCGGTGTCCCCACAGGATGAAACAG
CTGAAGTGGCAGTTCCAGCGGCTGTCCCTGCGGCAGAGGCTGAGGCCGAGGTGACGCTGCGGGAGCTC
CAGGAAGCCCTGGAGGAGGAGGTGCTCACCCGGCAGAGCCTGAGCCGGGAGATGGAGGCCATCCGCAC
GGACAACCAGAACTTCGCCAGTCAACTACGCGAGGCAGAGGCTCGGAACCGGGACCTAGAGGCACACG
TCCGGCAGTTGCAGGAGCGGATGGAGTTGCTGCAGGCAGAGGGAGCCACAGCTGTCACGGGGGTCCCC
AGTCCCCGGGCCACGGATCCACCTTCCCATATGGCCCCCCGGCCGTGGCTGTGGGCCAGTGCCCGCTG
GTGGGGCCAGGCCCCATGCACCGCCGCCACCTGCTGCTCCCTGCCAGGGTCCCTAGGCCTGGCCTATC
GGAGGCGCTTTCCCTGCTCCTGTTCGCCGTTGTTCTGTCTCGTGCCGCCGCCCTGGGCTGCATTGGGT
TGGTGGCCCACGCCGGCCAACTCACCGCAGTCTGGCGCCGCCCAGGAGCCGCCCGCGCTCCCTGAACC
CTAG

Promoters derived from DMPK may include that in www.ncbi.nlm.nih.gov/pubmed/9535904.

A codon optimized DMPK for human expression (see below) can also be used.

ATGGGTGGGCACTTTTGGCCGCCGGAACCCTACACGGTCTTTATGTGGGGCTCACCTTGGGAGGCAGA
CAGTCCTCGGGTGAAGTTGCGCGGCCGAGAGAAAGGCAGGCAAACGGAAGGTGGCGCTTTTCCTTTGG
TAAGTAGTGCCCTCTCTGGGGATCCTAGATTCTTTTCACCGACTACCCCTCCCGCGGAGCCTATAGTA
GTTAGATTGAAGGAGGTTCGACTTCAACGCGACGACTTCGAGATCCTGAAGGTTATAGGTCGGGGGGC
TTTCAGCGAGGTTGCGGTGGTAAAGATGAAGCAGACAGGTCAAGTGTACGCTATGAAGATTATGAACA
AGTGGGACATGCTTAAACGGGGAGAAGTCTCATGTTTCAGGGAGGAAAGAGATGTACTCGTAAACGGA
GATAGGCGGTGGATCACTCAGCTCCACTTCGCATTTCAAGATGAAAACTACTTGTACCTGGTAATGGA
GTACTATGTCGGAGGAGACCTTCTTACTCTTTTGTCCAAATTCGGCGAGCGCATTCCGGCAGAAATGG
CAAGATTCTACCTTGCAGAAATAGTGATGGCCATCGACAGTGTACACCGACTGGGCTACGTGCATAGG
GACATCAAACCGGACAACATTCTTCTTGATCGCTGCGGCCACATCCGGCTGGCCGATTTTGGCTCCTG
CCTCAAGTTGAGGGCGGATGGAACCGTCCGAAGTTTGGTGGCTGTAGGAACACCAGATTACTTGTCTC
CAGAAATATTGCAAGCCGTCGGAGGTGGCCCCGGGACCGGTTCATACGGTCCCGAGTGTGACTGGTGG
GCACTTGGCGTCTTTGCTTACGAAATGTTTTATGGGCAAACTCCCTTCTACGCAGATAGTACTGCAGA
AACCTATGGGAAGATAGTTCATTATAAGGAGCACCTCTCACTCCCATTGGTGGATGAAGGTGTTCCTG
AGGAAGCTCGCGATTTCATACAGCGGTTGCTTTGCCCCCCTGAGACGCGGCTGGGACGAGGGGGAGCA
GGAGACTTCCGCACCCATCCTTTCTTTTTCGGGTTGGATTGGGACGGTTTGAGAGACTCAGTACCTCC
ATTCACCCCCGACTTCGAAGGGGCAACGGATACATGCAACTTTGACCTTGTCGAGGACGGACTGACTG
CGATGGAAACACTGAGTGATATTCGCGAAGGTGCGCCCTTGGGTGTCCACTTGCCCTTCGTTGGGTAC
TCATATTCCTGCATGGCATTGAGGGATTCCGAGGTCCCAGGACCAACACCAATGGAGCTCGAAGCCGA
GCAGTTGCTTGAACCGCATGTGCAAGCGCCCAGCCTGGAGCCGTCTGTTAGTCCCCAGGACGAGACTG
CCGAGGTGGCAGTGCCTGCAGCTGTCCCGGCTGCAGAAGCAGAGGCAGAAGTCACGTTGCGGGAGTTG
CAGGAGGCACTCGAGGAGGAGGTCCTGACGCGCCAATCACTTAGCCGCGAAATGGAAGCAATCCGCAC
GGACAATCAGAACTTCGCGTCCCAACTGCGAGAAGCGGAAGCAAGAAATCGAGACTTGGAGGCACACG
TGCGCCAATTGCAAGAGCGCATGGAACTCCTCCAGGCAGAAGGTGCCACGGCTGTAACCGGGGTTCCC
AGTCCAAGAGCAACAGATCCTCCATCTCACATGGCACCACGGCCATGGCTGTGGGCCTCCGCCCGGTG
GTGGGGGCAAGCGCCATGCACAGCAGCTACTTGTTGTTCCTTGCCAGGATCTCTCGGTCTTGCCTATC
GAAGGCGCTTCCCTTGTAGTTGCTCCCCGCTCTTCTGTCTTGTCCCACCGCCTTGGGCAGCACTGGGT
TGGTGGCCTACTCCGGCAAACTCCCCACAGTCAGGTGCAGCCCAGGAACCACCAGCCCTCCCAGAGCC
GTAG

In certain embodiments, the antagonist encodes an RNAi reagent (such as siRNA, miRNA, shRNA etc) or antisense sequence that targets the mutant DMPK allele. The antagonist may be specific for the CTG repeat encoded the mutant allele, or be specific for another region of the mutant allele encoded mRNA (such as SNP present in the mutant allele).

In certain embodiments, a codon-optimized version of the wt allele may be expressed from one of the transcription cassettes, and RNA transcripts from such codon-optimized wt allele is not susceptible to the RNAi reagent.

In certain embodiments, the siRNA targeting locations can include the CTG repeat, 3′UTR, 5′UTR, and/or coding regions of DMPK, since DMPK gene to be expressed from the subject viral vectors can have codon optimized coding sequences and optimized UTRs that will be different from the natural DMPK gene sequence, and are thus not targeted by the siRNA against the mutant DMPK. A few representative shRNA sequences targeting mutant DMPK are provided below for illustration purpose.

>DM1-Repeat-shRNA-1 (the Second Line Represents the Passenger Strand, the Loop, and the Guide Strand Sequences):

GATCG
GCUGCUGCUGCUGCUGCU TCAAGAG AGCAGCAGCAGCAGCAGC
TTTTTTGAAGCT

>DM1-Repeat-shRNA-1 (the Second Line Represents the Passenger Strand, the Loop, and the Guide Strand Sequences):

GATCG
GCUGCUGCUGCUGCUGCU GCCCTGACCCAGC AGCAGCAGCAGCAGCAGC
TTTTTTGAAGCT

In a related aspect, instead of expressing a wt DMPK, a wt MBNL1 gene (e.g., a 1146-nts coding sequence) may be encoded as the first therapeutic agent.

For example, the wt MBNL1 gene can be encoded by a 1146 nts polynucleotide sequence (see below for one of the few MBNL1 isoforms available), and can be expressed either ubiquitously, or specifically in muscle using any one of muscle specific promoters such as CK8.

ATGGCTGTTAGTGTCACACCAATTCGGGACACAAAATGGCTAACACTGGAAGTATGTAGAGAGTTCCA
GAGGGGGACTTGCTCACGGCCAGACACGGAATGTAAATTTGCACATCCTTCGAAAAGCTGCCAAGTTG
AAAATGGACGAGTAATCGCCTGCTTTGATTCATTGAAAGGCCGTTGCTCCAGGGAGAACTGCAAATAT
CTTCATCCACCCCCACATTTAAAAACGCAGTTGGAGATAAATGGACGCAATAACTTGATTCAGCAGAA
GAACATGGCCATGTTGGCCCAGCAAATGCAACTAGCCAATGCCATGATGCCTGGTGCCCCATTACAAC
CCGTGCCAATGTTTTCAGTTGCACCAAGCTTAGCCACCAATGCATCAGCAGCCGCCTTTAATCCCTAT
CTGGGACCTGTTTCTCCAAGCCTGGTCCCGGCAGAGATCTTGCCGACTGCACCAATGTTGGTTACAGG
GAATCCGGGTGTCCCTGTACCTGCAGCTGCTGCAGCTGCTGCACAGAAATTAATGCGAACAGACAGAC
TTGAGGTATGTCGAGAGTACCAACGTGGCAATTGCAACCGAGGAGAAAATGATTGTCGGTTTGCTCAT
CCTGCTGACAGCACAATGATTGACACCAATGACAACACAGTCACTGTGTGTATGGATTACATCAAAGG
GAGATGCTCTCGGGAAAAGTGCAAATACTTTCATCCCCCTGCACATTTGCAAGCCAAGATCAAGGCTG
CCCAATACCAGGTCAACCAGGCTGCAGCTGCACAGGCTGCAGCCACCGCAGCTGCCATGACTCAGTCG
GCTGTCAAATCACTGAAGCGACCCCTCGAGGCAACCTTTGACCTGGGAATTCCTCAAGCTGTACTTCC
CCCATTACCAAAGAGGCCTGCTCTTGAAAAAACCAACGGTGCCACCGCAGTCTTTAACACTGGTATTT
TCCAATACCAACAGGCTCTAGCCAACATGCAGTTACAACAGCATACAGCATTTCTCCCACCAGGCTCA
ATATTGTGCATGACACCCGCTACAAGTGTTGTTCCCATGGTGCACGGTGCTACGCCAGCCACTGTGTC
CGCAGCAACAACATCTGCCACAAGTGTTCCCTTCGCTGCAACAGCCACAGCCAACCAGATACCCATAA
TATCTGCCGAACATCTGACTAGCCACAAGTATGTTACCCAGATGTAG

Promoters derived from MBNL1 may include that in www.ncbi.nlm.nih.gov/pmc/articles/PMC5389549/genes.

A codon optimized MBNL1 for human expression (see below) can also be used.
ATGGCAGTGAGCGTGACCCCGATAAGAGACACGAAGTGGCTGACTCTGGAGGTCTGCCGGGAATTTCA
GAGAGGTACATGTAGCAGACCGGACACAGAATGCAAGTTTGCTCATCCTAGCAAATCTTGTCAGGTCG
AGAACGGAAGGGTTATTGCGTGCTTCGACTCACTCAAAGGACGCTGCAGTCGCGAGAACTGCAAGTAC
CTTCATCCACCTCCGCACTTGAAAACGCAGCTTGAGATCAATGGGAGGAATAACCTTATACAACAAAA
GAATATGGCAATGCTTGCGCAACAGATGCAGCTGGCTAATGCCATGATGCCTGGGGCACCGCTGCAAC
CGGTTCCAATGTTCTCCGTTGCTCCTTCTCTGGCTACGAATGCTTCCGCAGCCGCCTTCAATCCTTAT
CTGGGCCCCGTTTCCCCAAGCCTGGTACCCGCAGAAATCCTGCCAACTGCTCCTATGTTGGTTACCGG
CAACCCCGGTGTGCCCGTTCCGGCCGCCGCAGCAGCAGCCGCCCAAAAACTTATGCGGACAGACAGGC
TGGAGGTGTGTCGGGAATATCAGCGCGGAAATTGCAATCGGGGAGAAAATGACTGTAGATTTGCCCAC
CCAGCAGACAGTACGATGATCGACACGAATGATAACACTGTAACCGTGTGCATGGATTATATTAAAGG
TAGGTGCAGCAGGGAGAAGTGCAAGTATTTTCATCCACCCGCCCATCTGCAAGCAAAGATCAAGGCGG
CACAATATCAAGTCAATCAAGCCGCTGCGGCGCAAGCCGCAGCGACAGCAGCCGCTATGACACAGTCA
GCCGTAAAGTCTCTGAAAAGGCCTTTGGAAGCCACTTTCGATTTGGGGATACCTCAAGCAGTACTTCC
ACCGCTGCCCAAGCGACCGGCGTTGGAAAAGACAAACGGTGCTACAGCAGTGTTCAATACCGGCATAT
TCCAATATCAGCAAGCACTCGCAAATATGCAGCTCCAGCAGCACACGGCTTTCCTTCCGCCAGGCTCA
ATCCTTTGCATGACTCCAGCTACATCAGTTGTACCGATGGTGCATGGAGCCACACCGGCGACTGTGTC
TGCCGCGACTACCTCAGCCACAAGCGTCCCCTTTGCGGCCACCGCCACTGCCAACCAGATACCGATTA
TTTCTGCAGAACACTTGACTTCACACAAGTATGTCACCCAAATGTAG

Yet another RED treatable by the vector of the invention is Huntington's Disease (HD), which is a fatal and currently uncurable autosomal dominant neurodegenerative disorder characterized by motor, cognitive, and behavioral impairment estimated to affect 1 in 10,000 in the US. It is caused by a toxic expansion in the CAG repeat region of the huntingtin (HTT) gene, which normally encodes a 3144-amino acid HTT protein. The resulting mutant huntingtin gene (mHTT) with more than 36 CAG repeats within exon 1 confers toxicity ascribed to the mutant protein through an as-yet unclear mechanism.

The mutant huntingtin protein has multiple deleterious molecular and cellular consequences, including loss of BDNF neurotrophic support for striatal neurons, impaired axonal transport, altered vesicle recycling, mitochondrial dysfunction, increased autophagy, protein aggregation, and transcriptional dysregulation. However, no single aberrant effect of mutant huntingtin explains neuronal dysfunction and early death. Patients with HD usually develop involuntary movements, cognitive dysfunction, and behavioral changes in the fourth decade of life.

The vector of the invention can be used to treat HD in at least two different approaches. In the first approach, a first therapeutic agent can be delivered using the vector of the invention to provide a normal or wild-type HTT gene, while a second therapeutic agent can be simultaneously delivered to specifically target the mutant HTT gene product.

For example, gene silencing through RNAi (siRNA, shRNA, miRNA etc) or ASO can be used to target the mutant HTT mRNA specifically. This can be achieved, through, for example, targeting SNP on the mutant HTT allele. van Bilsen et al. (Hum Gene Ther. 19(7):710-719, 2008) used a small interfering RNA (siRNA) that selectively reduces the endogenous mRNA for a heterozygous HD donor's pathogenic allele by approximately 80% by specifically targeting a single-nucleotide polymorphism (SNP) located several thousand bases downstream from the disease-causing mutation. Selective suppression of endogenous mutant HTT protein was also shown using this siRNA. Lombardi et al. (Exp Neurol. 217(2):312-319, 2009) genotyped DNA from 327 unrelated European Caucasian HD patients at 26 SNP sites in the HD gene, and found that over 86% of the patients were heterozygous for at least one SNP. Moreover, allele-specific siRNA targeting these sites are readily identifiable using a high throughput screening method, and that allele-specific siRNA identified using this method indeed show selective suppression of endogenous mutant htt protein in fibroblast cells from HD patients. Pfister et al. (Curr Biol. 19(9):774-8, 2009) similarly found that 48% of the tested patient population is heterozygous at a single SNP site; one isoform of this SNP is associated with HD. Further, five allele-specific siRNAs, corresponding to just three SNP sites, could be used to treat three-quarters of the United States and European HD patient populations.

In the second approach, instead of using a second therapeutic agent that specifically targets the mutant HTT gene product, a second therapeutic agent can simultaneously target both the mutant HTT gene product and the wild-type gene product to reduce (but not eliminate) the expression of both. Since wild-type huntingtin protein has numerous physiological activities in cells that are important for neuronal function, complete suppression of both mutant and wild-type huntingtin may not be desirable. Thus the simultaneous expression of wild-type HDD protein as the first therapeutic agent may further restore the wt HTT protein function. Since data has shown that reduction of mutant huntingtin protein expression, even just partially, may be sufficient for therapeutic benefit, this therapeutic approach can be used for patients not eligible for SNP-based mutant allele specific knock down of HTT expression.

As an alternative of these approaches, instead of expressing the wild-type HTT as the first therapeutic agent, a modifier beneficial to treat HD may be expressed as the first therapeutic agent. For example, Goold et al. (Hum Mol Genet. 28(4):650-661, 2019) showed that increased FAN1 (FANCD2- and FANCI-associated nuclease 1) expression is significantly associated with delayed HD age at onset, and slower progression of HD, suggesting FAN1 is protective in the context of an expanded HTT CAG repeat. FAN1 overexpression in human cells reduces CAG repeat expansion in exogenously expressed mutant HTT exon 1, and in patient-derived stem cells and differentiated medium spiny neurons, FAN1 knockdown increases CAG repeat expansion. The stabilizing effects are FAN1 concentration and CAG repeat length-dependent.

Another modifier beneficial to treat HD may be an antagonist of MSH3, since a variant of this mismatch repair gene MSH3 has been linked to HD as well as DM1 progression. See Flower et al. (Brain 142(7):1876-1886, 2019). The same strategy may also be used to treat DM1.

Yet another RED treatable by the vector of the invention is Friedreich's ataxia (FRDA) which is the most commonly inherited ataxia. It is an autosomal recessive genetic disease associated with degeneration of nerve tissue in the spinal cord which causes the ataxia. In the US, it is estimated to affect 1 in 50,000 individuals. Particularly affected are the sensory neurons essential for directing muscle movement of the arms and legs through connections with the cerebellum. Thus patients have difficulty walking, a loss of sensation in the arms and legs, and impaired speech that worsens over time. Many also have a form of heart disease called hypertrophic cardiomyopathy, which is the most common cause of death in FRDA patients. Currently, no effective treatment exists for this disease.

FRDA is caused by mutations in the FXN gene on Chr. 9, which produces an important protein called frataxin. Though the exact role of frataxin remains unclear, it is believed to assist iron-sulfur protein synthesis in the electron transport chain to generate ATP, and to regulate iron transfer in the mitochondria by providing a proper amount of reactive oxygen species (ROS) to maintain normal processes. Without frataxin, the energy in the mitochondria falls, and excess iron creates extra ROS, leading to further cell damage. Low frataxin levels lead to insufficient biosynthesis of iron-sulfur clusters that are required for mitochondrial electron transport and assembly of functional aconitase and iron dysmetabolism of the entire cell.

In 96% of the cases, the mutant FXN gene has 90-1,300 GAA trinucleotide repeat expansions in intron 1 of both alleles. This expansion causes epigenetic changes and formation of heterochromatin near the repeat. The length of the shorter GAA repeat is correlated with age of onset and disease severity. The formation of heterochromatin results in reduced transcription of the gene and low levels of frataxin—FDRA patients tend to have only 5-35% of the normal level of frataxin expressed in healthy individuals. Even heterozygous carriers of the mutant FXN gene have frataxin levels reduced by 50%; but this reduction is insufficient to cause symptoms. The remaining 4% of the cases are associated an GAA expansion in one allele, and a point mutation (missense, nonsense, or intronic point mutation) in the other.

Thus in certain embodiments, the RED is FRDA, wherein the first therapeutic agent comprises a coding sequence for wild-type FXN gene (e.g., a 630 nucleotide coding sequence), and (2) the second therapeutic agent comprises an antagonist specific for the mutant allele of FXN gene with the GAA repeats.

In certain embodiments, the second therapeutic agent comprises an RNAi reagent, such as an siRNA, shRNA, or miRNA coding sequence specific for the mutant allele of the FXN gene. Allele specificity can be based on expanded repeat of GAA, or an SNP associated with the mutant allele.

In certain embodiments, the vector of the invention may target the expression in a neuronal cell, such as neurons in the spinal cord or peripheral neurons, including a sensory neuron essential for directing muscle movement of the arms and legs. The vector may be locally delivered to the target neurons.

In certain embodiments, the vector of the invention may targeted the expression in the heart, muscle, pancreas, and/or other systems commonly affected in FRDA.

In certain embodiments, the vector of the invention may targeted the expression ubiquitously.

In certain embodiments, the vector of the invention uses a FXN promoter, a neuro-specific promoter (such as the synapsin promoter), a muscle-specific promoter (such as CK8), or a ubiquitous promoter to drive the expression of the first and/or the second therapeutic agent.

Yet another RED treatable by the vector of the invention is Fragile X syndrome (FXS), which is the most common cause of inherited mental retardation, intellectual disability, and autism, and is the second most common cause of genetically associated mental deficiencies, after trisomy 21. Conservative estimates report that Fragile X syndrome affects approximately 1 in 2,500-4,000 males and 1 in 7,000-8,000 females.

FXS is inherited in X-linked dominant pattern. It is typically due to an expansion of the CGG triplet repeat within the 5′-UTR region of the Fragile X mental retardation 1 (FMR1) gene on the X chromosome. Normal FMR1 gene has between 5 and 44 CGG repeats, most commonly 29 or 30 repeats. When this CGG repeat expands to 55 to more than 200, fragile X syndrome occurs. A premutation is said to be present when an intermediate number of repeats occurs. In individuals with a repeat expansion greater than 200, there is methylation of the CGG repeat expansion and FMR1 promoter, leading to the silencing of the FMR1 gene and a lack of its product. One study found that FMR1 silencing is mediated by the FMR1 mRNA, in that the FMR1 mRNA contains the transcribed CGG-repeat tract as part of the 5′ untranslated region, which hybridizes to the complementary CGG-repeat portion of the FMR1 gene to form an RNA·DNA duplex. The end result of having the mutant form of the FMR1 gene is that insufficient amount of the fragile X mental retardation protein (FMRP) is made, which protein is required for the normal development of connections between neurons. There is currently no cure for this disease.

Thus in certain embodiments, the RED is FXS, wherein the first therapeutic agent comprises a coding sequence for wild-type FMR1 gene (e.g., a 1863 nucleotide coding sequence), and (2) the second therapeutic agent comprises an antagonist specific for the mutant allele of FXS gene with the CCG repeats.

For example, the wt FMR1 gene can be encoded by a 1899 nts polynucleotide sequence (see below), and can be expressed in neurons using any one of neuron specific promoters such as the synapsin promoter.

ATGGAGGAGCTGGTGGTGGAAGTGCGGGGCTCCAATGGCGCTTTCTACAAGGCATTTGTAAAGGATGT
TCATGAAGATTCAATAACAGTTGCATTTGAAAACAACTGGCAGCCTGATAGGCAGATTCCATTTCATG
ATGTCAGATTCCCACCTCCTGTAGGTTATAATAAAGATATAAATGAAAGTGATGAAGTTGAGGTGTAT
TCCAGAGCAAATGAAAAAGAGCCTTGCTGTTGGTGGTTAGCTAAAGTGAGGATGATAAAGGGTGAGTT
TTATGTGATAGAATATGCAGCATGTGATGCAACTTACAATGAAATTGTCACAATTGAACGTCTAAGAT
CTGTTAATCCCAACAAACCTGCCACAAAAGATACTTTCCATAAGATCAAGCTGGATGTGCCAGAAGAC
TTACGGCAAATGTGTGCCAAAGAGGCGGCACATAAGGATTTTAAAAAGGCAGTTGGTGCCTTTTCTGT
AACTTATGATCCAGAAAATTATCAGCTTGTCATTTTGTCCATCAATGAAGTCACCTCAAAGCGAGCAC
ATATGCTGATTGACATGCACTTTCGGAGTCTGCGCACTAAGTTGTCTCTGATAATGAGAAATGAAGAA
GCTAGTAAGCAGCTGGAGAGTTCAAGGCAGCTTGCCTCGAGATTTCATGAACAGTTTATCGTAAGAGA
AGATCTGATGGGTCTAGCTATTGGTACTCATGGTGCTAATATTCAGCAAGCTAGAAAAGTACCTGGGG
TCACTGCTATTGATCTAGATGAAGATACCTGCACATTTCATATTTATGGAGAGGATCAGGATGCAGTG
AAAAAAGCTAGAAGCTTTCTCGAATTTGCTGAAGATGTAATACAAGTTCCAAGGAACTTAGTAGGCAA
AGTAATAGGAAAAAATGGAAAGCTGATTCAGGAGATTGTGGACAAGTCAGGAGTTGTGAGGGTGAGGA
TTGAGGCTGAAAATGAGAAAAATGTTCCACAAGAAGAGGAAATTATGCCACCAAATTCCCTTCCTTCC
AATAATTCAAGGGTTGGACCTAATGCCCCAGAAGAAAAAAAACATTTAGATATAAAGGAAAACAGCAC
CCATTTTTCTCAACCTAACAGTACAAAAGTCCAGAGGGTGTTAGTGGCTTCATCAGTTGTAGCAGGGG
AATCCCAGAAACCTGAACTCAAGGCTTGGCAGGGTATGGTACCATTTGTTTTTGTGGGAACAAAGGAC
AGCATCGCTAATGCCACTGTTCTTTTGGATTATCACCTGAACTATTTAAAGGAAGTAGACCAGTTGCG
TTTGGAGAGATTACAAATTGATGAGCAGTTGCGACAGATTGGAGCTAGTTCTAGACCACCACCAAATC
GTACAGATAAGGAAAAAAGCTATGTGACTGATGATGGTCAAGGAATGGGTCGAGGTAGTAGACCTTAC
AGAAATAGGGGGCACGGCAGACGCGGTCCTGGATATACTTCAGGAACTAATTCTGAAGCATCAAATGC
TTCTGAAACAGAATCTGACCACAGAGACGAACTCAGTGATTGGTCATTAGCTCCAACAGAGGAAGAGA
GGGAGAGCTTCCTGCGCAGAGGAGACGGACGGCGGCGTGGAGGGGGAGGAAGAGGACAAGGAGGAAGA
GGACGTGGAGGAGGCTTCAAAGGAAACGACGATCACTCCCGAACAGATAATCGTCCACGTAATCCAAG
AGAGGCTAAAGGAAGAACAACAGATGGATCCCTTCAGATCAGAGTTGACTGCAATAATGAAAGGAGTG
TCCACACTAAAACATTACAGAATACCTCCAGTGAAGGTAGTCGGCTGCGCACGGGTAAAGATCGTAAC
CAGAAGAAAGAGAAGCCAGACAGCGTGGATGGTCAGCAACCACTCGTGAATGGAGTACCCTAA

The synapsin promoter can be found at www.ncbi.nlm.nih.gov/pmc/articles/PMC4229583/). Alternatively, natural FMR1 promoter or U6 promoter may be used to drive the expression.

A codon optimized FMR1 for human expression (see below) can also be used.

ATGGAGGAGTTGGTTGTGGAGGTACGAGGTTCAAACGGTGCTTTTTATAAGGCGTTCGTAAAGGACGT
ACACGAAGACTCAATAACAGTTGCATTCGAGAATAATTGGCAACCTGATCGACAGATCCCATTTCACG
ACGTGAGGTTTCCACCCCCAGTGGGCTATAATAAGGATATCAACGAGTCTGATGAGGTGGAGGTATAT
TCTCGGGCGAATGAGAAAGAGCCGTGTTGCTGGTGGCTCGCTAAAGTTCGGATGATCAAGGGTGAGTT
TTATGTCATAGAATATGCAGCTTGTGATGCTACCTATAACGAAATCGTCACAATTGAGAGGCTCAGAT
CAGTTAATCCGAACAAACCCGCTACTAAAGATACGTTTCACAAGATCAAACTCGACGTCCCTGAGGAC
CTTAGGCAAATGTGTGCGAAAGAAGCAGCTCATAAAGACTTTAAGAAAGCAGTTGGAGCATTCAGCGT
TACGTATGATCCTGAAAATTACCAGTTGGTCATCCTGTCTATCAACGAGGTTACGAGCAAAAGGGCGC
ATATGCTGATTGATATGCATTTCCGGAGCTTGCGCACGAAATTGTCTTTGATCATGAGGAACGAGGAA
GCGTCAAAGCAGCTCGAAAGTAGCCGGCAGCTTGCCTCAAGATTTCATGAACAGTTTATCGTGCGGGA
AGACCTTATGGGCCTGGCGATCGGCACGCACGGGGCTAATATTCAGCAGGCCCGGAAAGTGCCTGGTG
TCACCGCTATCGACCTGGATGAAGACACTTGCACCTTTCACATTTATGGTGAGGACCAGGACGCGGTG
AAAAAAGCTAGGAGTTTTCTCGAGTTCGCCGAGGACGTTATACAAGTTCCCAGGAACCTTGTAGGCAA
AGTAATAGGTAAGAATGGGAAACTCATACAAGAGATAGTCGATAAAAGTGGCGTAGTCAGAGTAAGGA
TCGAGGCGGAAAATGAGAAAAATGTACCCCAAGAGGAAGAAATCATGCCACCAAACAGTTTGCCGTCT
AACAACAGTCGGGTCGGCCCCAATGCGCCGGAGGAAAAAAAGCATCTTGATATTAAAGAAAATAGCAC
ACACTTCTCACAACCCAATTCAACCAAAGTGCAACGAGTTCTTGTAGCTTCATCAGTCGTTGCCGGCG
AATCTCAAAAACCAGAACTCAAAGCCTGGCAAGGTATGGTCCCATTCGTATTTGTGGGTACGAAGGAC
TCTATCGCCAACGCCACCGTACTCCTCGACTACCACCTGAACTACCTGAAGGAAGTTGATCAGCTTCG
CCTCGAGCGCTTGCAAATAGACGAGCAGTTGCGGCAGATAGGAGCAAGCAGTAGGCCCCCACCAAACA
GGACAGATAAAGAAAAAAGCTACGTTACGGATGACGGTCAAGGAATGGGCCGCGGCAGCCGACCATAT
AGGAATCGCGGGCATGGTCGCAGAGGACCCGGTTACACCTCAGGCACCAACAGCGAGGCCTCAAACGC
CTCAGAAACGGAGAGCGACCATAGGGATGAACTTTCAGACTGGTCCTTGGCTCCGACTGAGGAGGAAC
GGGAGAGTTTCTTGCGGAGAGGTGATGGCCGCAGGAGAGGGGGTGGGGGGCGAGGTCAAGGAGGACGG
GGCAGAGGAGGCGGATTTAAGGGCAACGATGATCACTCAAGGACCGATAATAGGCCAAGAAATCCGCG
CGAAGCGAAAGGGAGGACTACAGACGGGAGCTTGCAAATAAGAGTCGATTGCAACAATGAGCGGTCCG
TGCACACAAAAACCCTTCAAAATACCTCATCCGAGGGTTCCCGCCTGCGAACAGGCAAAGACAGGAAT
CAAAAGAAAGAGAAACCAGATTCAGTCGACGGACAACAACCTTTGGTTAACGGTGTGCCCTAA

In certain embodiments, the second therapeutic agent comprises an RNAi reagent, such as an siRNA, shRNA, or miRNA coding sequence specific for the mutant allele of the FMR1 gene. Allele specificity can be based on expanded repeat of CCG, or an SNP associated with the mutant allele.

In certain embodiments, the siRNA targeting locations can include the CGG repeat, 3′UTR, 5′UTR, and/or coding regions of FMR1, since FMR1 gene to be expressed from the subject viral vectors can have codon optimized coding sequences and optimized UTRs that will be different from the natural FMR1 gene sequence, and are thus not targeted by the siRNA against the mutant FMR1. A few representative shRNA sequences targeting mutant FMR1 are provided below for illustration purpose.

>FMR1-siRNA-1 (the Second Line Represents the Passenger Strand, the Loop, and the Guide Strand Sequences):

GATCG
CAATTTTCAGATTTGCACAAA TCAAGAG TTTGTGCAAATCTGAAAATTG
TTTTTTGAAGCT

>FMR1-siRNA-2 (the Second Line Represents the Passenger Strand, the Loop, and the Guide Strand Sequences):

GATCG
CAATTTTCAGATTTGCACAAA GCCCTGACCCAGC
TTTGTGCAAATCTGAAAATTG TTTTTTGAAGCT

>FMR1-siRNA-3 (Mir-30 Backbone) (the Second Line Represents the Passenger Strand, the Loop, and the Guide Strand Sequences):

AACAGAAGGCTCGAGAAGGTATATTGCTGTTGACAGTGAGCG
CAATTTTCAGATTTGCACAAA TAGTGAAGCCACAGATGTA
TTTGTGCAAATCTGAAAATTG
TGCCTACTGCCTCGGACTTCAAGGGGCTAGAATTCGA

In certain embodiments, the vector of the invention may target the expression in a neuronal cell. The vector may be locally delivered to the target neurons.

In certain embodiments, the vector of the invention may targeted the expression ubiquitously.

In certain embodiments, the vector of the invention uses a FMR1 promoter, a neuro-specific promoter (such as the synapsin promoter), or a ubiquitous promoter to drive the expression of the first and/or the second therapeutic agent.

### Composition and Pharmaceutical Composition

In another embodiment, the invention contemplates compositions comprising rAAV of the present invention. Compositions of the invention comprise rAAV and a pharmaceutically acceptable carrier. The compositions may also comprise other ingredients such as diluents and adjuvants. Acceptable carriers, diluents and adjuvants are nontoxic to recipients and are preferably inert at the dosages and concentrations employed, and include buffers such as phosphate, citrate, or other organic acids; antioxidants such as ascorbic acid; low molecular weight polypeptides; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counter ions such as sodium; and/or nonionic surfactants such as Tween, pluronics or polyethylene glycol (PEG).

### Dosing and Administration

Titers of rAAV to be administered in methods of the invention will vary depending, for example, on the particular rAAV, the mode of administration, the treatment goal, the individual, and the cell type(s) being targeted, and may be determined by methods standard in the art. Titers of rAAV may range from about 1×10⁶, about 1×10⁷, about 1×10⁸, about 1×10⁹, about 1×10¹⁰, about 1×10¹¹, about 1×10¹², about 1×10¹³, to about 1×10¹⁴ or more DNase resistant particles (DRP) per ml. Dosages may also be expressed in units of viral genomes (vg).

Methods of transducing a target cell with rAAV, in vivo or in vitro, are contemplated by the invention. The in vivo methods comprise the step of administering an effective dose, or effective multiple doses, of a composition comprising a rAAV of the invention to an animal (including a human being) in need thereof. If the dose is administered prior to development of a disorder/disease, the administration is prophylactic. If the dose is administered after the development of a disorder/disease, the administration is therapeutic. In embodiments of the invention, an effective dose is a dose that alleviates (eliminates or reduces) at least one symptom associated with the disorder/disease state being treated, that slows or prevents progression to a disorder/disease state, that slows or prevents progression of a disorder/disease state, that diminishes the extent of disease, that results in remission (partial or total) of disease, and/or that prolongs survival. An example of a disease contemplated for prevention or treatment with methods of the invention is PMD or other disease characterized by defects in myelin production, degeneration, regeneration, or function.

For administration, effective amounts and therapeutically effective amounts (also referred to herein as doses) may be initially estimated based on results from in vitro assays and/or animal model studies. For example, a dose may be formulated in animal models to achieve a circulating concentration range that includes the IC₅₀ as determined in cell culture. Such information may be used to more accurately determine useful doses in subjects of interest.

Administration of an effective dose of the compositions may be by routes standard in the art including, but not limited to, intramuscular, parenteral, intravenous, oral, buccal, nasal, pulmonary, intracranial, intraosseous, intraocular, rectal, or vaginal. Route(s) of administration and serotype(s) of AAV components of the rAAV (in particular, the AAV ITRs and capsid protein) of the invention may be chosen and/or matched by those skilled in the art taking into account the infection and/or disease state being treated and the target cells/tissue(s) that are to express the one or more coding sequences and/or micro-dystrophin.

Specifically, the formulations described herein may be administered by, without limitation, injection, infusion, perfusion, inhalation, lavage, and/or ingestion. Routes of administration may include, but are not limited to, intravenous, intradermal, intraarterial, intraperitoneal, intralesional, intracranial, intraarticular, intraprostatic, intrapleural, intratracheal, intranasal, intravitreal, intravaginal, intrarectal, topically, intratumoral, intramuscular, intravesicular, intrapericardial, intraumbilical, intraocularal, mucosal, oral, subcutaneous, and/or subconjunctival.

The invention provides for local administration or systemic administration of an effective dose of rAAV and compositions of the invention including combination therapy of the invention. For example, systemic administration is administration into the circulatory system so that the entire body is affected. Systemic administration includes enteral administration such as absorption through the gastrointestinal tract and parental administration through injection, infusion or implantation.

In particular, actual administration of rAAV of the present invention may be accomplished by using any physical method that will transport the rAAV recombinant vector into the target tissue of an animal, such as the skeletal muscles. Administration according to the invention includes, but is not limited to, injection into muscle, the bloodstream and/or directly into the liver. Simply re-suspending a rAAV in phosphate buffered saline has been demonstrated to be sufficient to provide a vehicle useful for muscle tissue expression, and there are no known restrictions on the carriers or other components that can be co-administered with the rAAV (although compositions that degrade DNA should be avoided in the normal manner with rAAV). Capsid proteins of a rAAV may be modified so that the rAAV is targeted to a particular target tissue of interest such as muscle. See, for example, WO 02/053703, the disclosure of which is incorporated by reference herein.

Pharmaceutical compositions can be prepared as injectable formulations or as topical formulations to be delivered to the muscles by transdermal transport. Numerous formulations for both intramuscular injection and transdermal transport have been previously developed and can be used in the practice of the invention. The rAAV can be used with any pharmaceutically acceptable carrier for ease of administration and handling.

The dose of rAAV to be administered in methods disclosed herein will vary depending, for example, on the particular rAAV, the mode of administration, the treatment goal, the individual, and the cell type(s) being targeted, and may be determined by methods standard in the art.

The actual dose amount administered to a particular subject may also be determined by a physician, a veterinarian, or a researcher, taking into account parameters such as, but not limited to, physical and physiological factors including body weight, severity of condition, type of disease, previous or concurrent therapeutic interventions, idiopathy of the subject, and/or route of administration.

Titers of each rAAV administered may range from about 1×10⁶, about 1×10⁷, about 1×10⁸, about 1×10⁹, about 1×10¹⁰, about 1×10¹¹, about 1×10¹², about 1×10¹³, about 1×10¹⁴, or to about 1×10¹⁵ or more DNase resistant particles (DRP) per ml. Dosages may also be expressed in units of viral genomes (vg) (i.e., 1×10⁷ vg, 1×10⁸ vg, 1×10⁹ vg, 1×10¹⁰ vg, 1×10¹¹ vg, 1×10¹² vg, 1×10¹³ vg, 1×10¹⁴ vg, 1×10¹⁵ vg, respectively). Dosages may also be expressed in units of viral genomes (vg) per kilogram (kg) of bodyweight (i.e., 1×10¹⁰ vg/kg, 1×10¹¹ vg/kg, 1×10¹² vg/kg, 1×10¹³ vg/kg, 1×10¹⁴ vg/kg, 1×10¹⁵ vg/kg respectively). Methods for titering AAV are described in Clark et al., Hum. Gene Ther. 10:1031-1039, 1999.

Exemplary doses may range from about 1×10¹⁰ to about 1×10¹⁵ vector genomes (vg)/kilogram of body weight. In some embodiments, doses may comprise 1×10¹⁰ vg/kg of body weight, 1×10¹¹ vg/kg of body weight, 1×10¹² vg/kg of body weight, 1×10¹³ vg/kg of body weight, 1×10¹⁴ vg/kg of body weight, or 1×10¹⁵ vg/kg of body weight. Doses may comprise 1×10¹⁰ vg/kg/day, 1×10¹¹ vg/kg/day, 1×10¹² vg/kg/day, 1×10¹³ vg/kg/day, 1×10¹⁴ vg/kg/day, or 1×10¹⁵ vg/kg/day. Doses may range from 0.1 mg/kg/day to 5 mg/kg/day or from 0.5 mg/kg/day to 1 mg/kg/day or from 0.1 mg/kg/day to 5 μg/kg/day or from 0.5 mg/kg/day to 1 μg/kg/day. In other non-limiting examples, a dose may comprise 1 μg/kg/day, 5 μg/kg/day, 10 μg/kg/day, 50 μg/kg/day, 100 μg/kg/day, 200 μg/kg/day, 350 μg/kg/day, 500 μg/kg/day, 1 mg/kg/day, 5 mg/kg/day, 10 mg/kg/day, 50 mg/kg/day, 100 mg/kg/day, 200 mg/kg/day, 350 mg/kg/day, 500 mg/kg/day, or 1000 mg/kg/day. Therapeutically effective amounts may be achieved by administering single or multiple doses during the course of a treatment regimen (i.e., days, weeks, months, etc.).

In some embodiments, the pharmaceutical composition is in a dosage form of 10 mL of aqueous solution having at least 1.6×10¹³ vector genomes. In some embodiments, the dosage has a potency of at least 2×10¹² vector genomes per milliliter. In some embodiments, the dosage comprises a sterile aqueous solution comprising 10 mM L-histidine at pH 6.0, 150 mM sodium chloride, and 1 mM magnesium chloride. In some embodiments, the pharmaceutical composition is in a dosage form of 10 mL of a sterile aqueous solution comprising 10 mM L-histidine at pH 6.0, 150 mM sodium chloride, and 1 mM magnesium chloride; and having at least 1.6×10¹³ vector genomes.

In some embodiments, the pharmaceutical composition may be a dosage comprising between 1×10¹⁰ and 1×10¹⁵ vector genomes in 10 mL aqueous solution; between 1×10¹¹ and 1×10¹⁴ vector genomes in 10 mL aqueous solution; between 1×10¹² and 2×10¹³ vector genomes in 10 mL aqueous solution; or greater than or equal to about 1.6×10¹³ vector genomes in 10 mL aqueous solution. In some embodiments the aqueous solution is a sterile aqueous solution comprises about 10 mM L-histidine pH 6.0, with 150 mM sodium chloride, and 1 mM magnesium chloride. In some embodiments, the dosage has a potency of greater than about 1×10¹¹ vector genomes per milliliter (vg/mL), greater than about 1×10¹² vg/mL, greater than about 2×10¹² vg/mL, greater than about 3×10¹² vg/mL, or greater than about 4×10¹² vg/mL.

In some embodiments, at least one AAV vector is provided as part of a pharmaceutical composition. The pharmaceutical composition may comprise, for example, at least 0.1% w/v of the AAV vector. In some other embodiments, the pharmaceutical composition may comprise between 2% to 75% of compound per weight of the pharmaceutical composition, or between 25% to 60% of compound per weight of the pharmaceutical composition.

In some embodiments, the dosage is in a kit. The kit may further include directions for use of the dosage.

For purposes of intramuscular injection, solutions in an adjuvant such as sesame or peanut oil or in aqueous propylene glycol can be employed, as well as sterile aqueous solutions. Such aqueous solutions can be buffered, if desired, and the liquid diluent first rendered isotonic with saline or glucose. Solutions of rAAV as a free acid (DNA contains acidic phosphate groups) or a pharmacologically acceptable salt can be prepared in water suitably mixed with a surfactant such as hydroxpropylcellulose. A dispersion of rAAV can also be prepared in glycerol, liquid polyethylene glycols and mixtures thereof and in oils. Under ordinary conditions of storage and use, these preparations contain a preservative to prevent the growth of microorganisms. In this connection, the sterile aqueous media employed are all readily obtainable by standard techniques well-known to those skilled in the art.

In some embodiments, for injection, formulations may be made as aqueous solutions, such as in buffers including, but not limited to, Hanks' solution, Ringer's solution, and/or physiological saline. The solutions may contain formulatory agents such as suspending, stabilizing, and/or dispersing agents. Alternatively, the formulation may be in lyophilized and/or powder form for constitution with a suitable vehicle control (e.g., sterile pyrogen-free water) before use.

Any formulation disclosed herein may advantageously comprise any other pharmaceutically acceptable carrier or carriers which comprise those that do not produce significantly adverse, allergic, or other untoward reactions that may outweigh the benefit of administration, whether for research, prophylactic, and/or therapeutic treatments. Exemplary pharmaceutically acceptable carriers and formulations are disclosed in Remington's Pharmaceutical Sciences, 18th Ed., Mack Printing Company, 1990, which is incorporated by reference herein for its teachings regarding the same. Moreover, formulations may be prepared to meet sterility, pyrogenicity, general safety, and purity standards as required by the United States FDA's Division of Biological Standards and Quality Control and/or other relevant U.S. and foreign regulatory agencies.

Exemplary, generally used pharmaceutically acceptable carriers may comprise, but are not limited to, bulking agents or fillers, solvents or co-solvents, dispersion media, coatings, surfactants, antioxidants (e.g., ascorbic acid, methionine, and vitamin E), preservatives, isotonic agents, absorption delaying agents, salts, stabilizers, buffering agents, chelating agents (e.g., EDTA), gels, binders, disintegration agents, and/or lubricants.

Exemplary buffering agents may comprise, but are not limited to, citrate buffers, succinate buffers, tartrate buffers, fumarate buffers, gluconate buffers, oxalate buffers, lactate buffers, acetate buffers, phosphate buffers, histidine buffers, and/or trimethylamine salts.

Exemplary preservatives may comprise, but are not limited to, phenol, benzyl alcohol, meta-cresol, methylparaben, propyl paraben, octadecyldimethylbenzyl ammonium chloride, benzalkonium halides, hexamethonium chloride, alkyl parabens (such as methyl or propyl paraben), catechol, resorcinol, cyclohexanol, and/or 3-pentanol.

Exemplary isotonic agents may comprise polyhydric sugar alcohols comprising, but not limited to, trihydric or higher sugar alcohols, (e.g., glycerin, erythritol, arabitol, xylitol, sorbitol, and/or mannitol).

Exemplary stabilizers may comprise, but are not limited to, organic sugars, polyhydric sugar alcohols, polyethylene glycol, sulfur-containing reducing agents, amino acids, low molecular weight polypeptides, proteins, immunoglobulins, hydrophilic polymers, and/or polysaccharides.

Formulations may also be depot preparations. In some embodiments, such long-acting formulations may be administered by, without limitation, implantation (e.g., subcutaneously or intramuscularly) or by intramuscular injection. Thus, for example, compounds may be formulated with suitable polymeric and/or hydrophobic materials (e.g., as an emulsion in an acceptable oil) or ion exchange resins, or as sparingly soluble derivatives (e.g., as a sparingly soluble salt).

Additionally, in various embodiments, the AAV vectors may be delivered using sustained-release systems, such as semipermeable matrices of solid polymers comprising the AAV vector. Various sustained-release materials have been established and are well known by those of ordinary skill in the art. Sustained-release capsules may, depending on their chemical nature, release the vector following administration for a few weeks up to over 100 days.

The pharmaceutical carriers, diluents or excipients suitable for injectable use include sterile aqueous solutions or dispersions and sterile powders for the extemporaneous preparation of sterile injectable solutions or dispersions. In all cases the form must be sterile and must be fluid to the extent that easy syringability exists. It must be stable under the conditions of manufacture and storage and must be preserved against the contaminating actions of microorganisms such as bacteria and fungi. The carrier can be a solvent or dispersion medium containing, for example, water, ethanol, polyol (for example, glycerol, propylene glycol, liquid polyethylene glycol and the like), suitable mixtures thereof, and vegetable oils. The proper fluidity can be maintained, for example, by the use of a coating such as lecithin, by the maintenance of the required particle size in the case of a dispersion and by the use of surfactants. The prevention of the action of microorganisms can be brought about by various antibacterial and antifungal agents, for example, parabens, chlorobutanol, phenol, sorbic acid, thimerosal and the like. In many cases it will be preferable to include isotonic agents, for example, sugars or sodium chloride. Prolonged absorption of the injectable compositions can be brought about by use of agents delaying absorption, for example, aluminum monostearate and gelatin.

Sterile injectable solutions are prepared by incorporating rAAV in the required amount in the appropriate solvent with various other ingredients enumerated above, as required, followed by filter sterilization. Generally, dispersions are prepared by incorporating the sterilized active ingredient into a sterile vehicle which contains the basic dispersion medium and the required other ingredients from those enumerated above. In the case of sterile powders for the preparation of sterile injectable solutions, the preferred methods of preparation are vacuum drying and the freeze drying technique that yield a powder of the active ingredient plus any additional desired ingredient from the previously sterile-filtered solution thereof.

Transduction with rAAV may also be carried out in vitro. In one embodiment, desired target muscle cells are removed from the subject, transduced with rAAV and reintroduced into the subject. Alternatively, syngeneic or xenogeneic muscle cells can be used where those cells will not generate an inappropriate immune response in the subject.

Suitable methods for the transduction and reintroduction of transduced cells into a subject are known in the art. In one embodiment, cells can be transduced in vitro by combining rAAV with muscle cells, e.g., in appropriate media, and screening for those cells harboring the DNA of interest using conventional techniques such as Southern blots and/or PCR, or by using selectable markers. Transduced cells can then be formulated into pharmaceutical compositions, and the composition introduced into the subject by various techniques, such as by intramuscular, intravenous, subcutaneous and intraperitoneal injection, or by injection into smooth and cardiac muscle, using e.g., a catheter.

Transduction of cells with rAAV of the invention results in sustained co-expression of said one or more additional coding sequences and micro-dystrophin. The present invention thus provides methods of administering/delivering rAAV which co-expresses said one or more additional coding sequences and micro-dystrophin to an animal, preferably a human being. These methods include transducing tissues (including, but not limited to, tissues such as muscle, organs such as liver and brain, and glands such as salivary glands) with one or more rAAV of the present invention. Transduction may be carried out with gene cassettes comprising tissue specific control elements. For example, one embodiment of the invention provides methods of transducing muscle cells and muscle tissues directed by muscle specific control elements, including, but not limited to, those derived from the actin and myosin gene families, such as from the myoD gene family (See Weintraub et al., Science 251:761-766, 1991), the myocyte-specific enhancer binding factor MEF-2 (Cserjesi and Olson, Mol Cell Biol 11:4854-4862, 1991), control elements derived from the human skeletal actin gene (Muscat et al., Mol Cell Biol 7:4089-4099, 1987), the cardiac actin gene, muscle creatine kinase sequence elements (Johnson et al., Mol Cell Biol 9:3393-3399, 1989), and the murine creatine kinase enhancer (mCK) element, control elements derived from the skeletal fast-twitch troponin C gene, slow-twitch cardiac troponin C gene and the slow-twitch troponin I gene: hypoxia-inducible nuclear factors (Semenza et al., Proc Natl Acad Sci U.S.A. 88:5680-5684, 1991), steroid-inducible elements and promoters including the glucocorticoid response element (GRE) (See Mader and White, Proc. Natl. Acad. Sci. U.S.A. 90:5603-5607, 1993), and other control elements.

Muscle tissue is an attractive target for in vivo DNA delivery, because it is not a vital organ and is easy to access. The invention contemplates sustained co-expression of miRNAs and micro-dystrophin from transduced myofibers.

As used herein, “muscle cell” or “muscle tissue” is meant a cell or group of cells derived from muscle of any kind (for example, skeletal muscle and smooth muscle, e.g., from the digestive tract, urinary bladder, blood vessels or cardiac tissue). Such muscle cells may be differentiated or undifferentiated, such as myoblasts, myocytes, myotubes, cardiomyocytes and cardiomyoblasts.

The term “transduction” is used to refer to the administration/delivery of the one or more additional coding sequences and the coding region of the micro-dystrophin to a recipient cell either in vivo or in vitro, via a replication-deficient rAAV of the invention resulting in co-expression of the one or more additional coding sequences and micro-dystrophin by the recipient cell.

Thus, the invention provides methods of administering an effective dose (or doses, administered essentially simultaneously or doses given at intervals) of rAAV that encode said one or more additional coding sequences and micro-dystrophin to a patient in need thereof.

### AAV Production

Genes encoding the necessary replication (rep) and structural (cap) proteins of AAV vectors have been deleted from AAV vectors to allow insertion of the sequences to be delivered between the remaining terminal repeat sequences. Thus for growth of AAV vectors, not only is a helper virus required, but the genes encoding the rep and cap proteins need to be delivered to infected cells. Alternatively, the genes encoding the rep and cap proteins need to be present in the cells used for production.

AAV vectors suitable for the methods of the invention can be produced using any of the art-recognized methods. In a recent review, Penaud-Budloo et al. (Molecular Therapy: Methods & Clinical Development Vol. 8, pages 166-180, 2018) provided a review of the most commonly used upstream methods to produce rAAVs. Each methods described therein are incorporated herein by reference.

### Transient Transfection of Packaging Cell Line (HEK293)

In particular, in certain embodiments, the AAV vector is produced using transient transfection of a packaging cell line such as HEK293 cells. This is the most established AAV production method comprising plasmid transfection of human embryonic HEK293 cells. Typically, HEK293 cells are simultaneously transfected by a vector plasmid (containing the gene of interest, such as the subject polynucleotide encoding both the dystrophin minigene and the one or more additional coding sequences), and one or two helper plasmids, using calcium phosphate or polyethylenimine (PEI), a cationic polymer.

The helper plasmid(s) allow the expression of the four Rep proteins, the three AAV structural proteins VP1, VP2, and VP3, the AAP, and the adenoviral auxiliary functions E2A, E4, and VARNA. The additional adenoviral E1A/E1B co-factors necessary for rAAV replication are ex-pressed in HEK293 producer cells. Rep-cap and adenoviral helper sequences are either cloned on two separate plasmids or combined on one plasmid, hence both a triple plasmid system and a two plasmid system for transfection are possible. The triple plasmid protocol lends versatility with a cap gene that can easily be switched from one serotype to another.

The plasmids are usually produced by conventional techniques in E. coli using bacterial origin and anti-biotic-resistance gene or by minicircle technology.

Transient transfection in adherent HEK293 cells has been used for large-scale manufacturing of rAAV vectors. Recently, HEK293 cells have also been adapted to suspension conditions to be economically viable in the long term.

HEK293 lines are usually propagated in DMEM completed with L-glutamine, 5%-10% of fetal bovine serum (FBS), and 1% penicillin-streptomycin, except for suspension HEK293 cells that are maintained in serum-free suspension F17, Expi293, or other manufacturer-specific media. For adherent cells, the percentage of FBS can be reduced during AAV production in order to limit contamination by animal-derived components.

Generally, the rAAV vectors are recovered 48-72 hr after plasmid transfection from the cell pellet and/or supernatant, depending on the serotype.

Infection of Insect Cells with Recombinant Baculovirus

The baculovirus-Sf9 platform has been established as a GMP-compatible and scalable alternative AAV production method in mammalian cells. It can generate up to 2×10⁵ vector genomes (vg) per cell in crude harvests.

Current protocol involves infection of the Sf9 insect cells with two recombinant baculoviruses a baculovirus expression vector (BEV) allowing the synthesis of Rep78/52 and Caps, and a recombinant baculovirus carrying the gene of interest flanked by the AAV ITRs. Several serum-free media are adapted for Sf9 cell growth in suspension.

The dual-baculovirus-Sf9 production system has many advantages over other production platforms regarding these safety issues: (1) the use of serum-free media; (2) despite the discovery of adventitious virus transcripts in Sf cell lines, most of the viruses infecting insects do not replicate actively in mammalian cells; and (3) no helper virus is required for rAAV production in insect cells besides baculovirus.

In certain embodiments, stable Sf9 insect cell lines expressing Rep and Cap proteins are used, thus requiring the infection of only one recombinant baculovirus for the production of infectious rAAV vectors at high yield.

Infection of Mammalian Cells with rHSV Vectors

HSV is a helper virus for replication of AAV in permissive cells. Thus, the HSV can serve both as a helper and as a shuttle to deliver the necessary AAV functions that support AAV genome replication and packaging to the producing cells.

AAV production based on co-infection with rHSV can efficiently generate a large amount of rAAV. In addition to high overall yields (up to 1.5×10⁵ vg/cell), the method is further advantageous in that it creates rAAV stocks with apparently increased quality as measured by an improved viral potency.

In this method, cells, typically the hamster BHK21 cell line or the HEK293 and derivatives, are infected with two rHSVs, one carrying the gene of interest bracketed by AAV ITR (rHSV-AAV), and the second with the AAV rep and cap ORFs of the desired serotype (rHSVrepcap). After 2-3 days, the cells and/or the media are collected, and rAAV is purified over multiple purification steps to remove cellular impurities, HSV-derived contaminants, and unpackaged AAV DNA.

Thus in some embodiments, HSV serves as a helper virus for AAV infection. In some embodiments, AAV growth is accomplished using non-replicating mutants of HSV with ICP27 deleted.

Certain methods for producing recombinant AAV viral particles in a mammalian cell have been known in the art and improved over the past decade. For example, U.S. Application Publication No. 20070202587 describes recombinant AAV production in mammalian cells based on co-infection of the cells with two or more replication-defective recombinant HSV vectors. U.S. Application Publication No. 20110229971 and Thomas et al. (Hum. Gene Ther. 20(8):861-870, 2009) describes a scalable recombinant AAV production method using recombinant HSV type 1 coinfection of suspension-adapted mammalian cells. Adamson-Small et al. (Hum. Gene Ther. Methods 28(1):1-14, 2017) describes an improved AAV production method in a serum-free suspension manufacturing platform using the HSV system.

### Mammalian Stable Cell Lines

rAAV vectors can also be efficiently and scalably produced using stable mammalian producer cells stably expressing rep and cap genes. Such cells can be infected by wild-type Ad5 helper virus (which is genetically stable and can be easily produced at high titers) to induce high-level expression of rep and cap. Infectious rAAV vectors can be generated upon infection of these packaging cells lines with wild-type Ad type 5, and providing the rAAV genome by either plasmid transfection or after infection with a recombinant Ad/AAV hybrid virus.

Alternatively, Ad can be replaced by HSV-1 as the helper virus.

Suitable stable mammalian producer cells may include HeLa-derived producer cell lines, A549 cells, or HEK293 cells. A preferred HeLa cell line is HeLaS3 cells, a suspension adapted HeLa subclone.

The methods herein described can be used to manufacture the subject AAV vectors in animal components-free medium, preferably at 250-L scale, or 2,000-L commercial scale.

### EXAMPLES

### Example 1 In Vitro Expression of Coding Sequences from Divergent Constructs

The divergent viral vectors of the invention are capable of expressing not only the functional gene or protein of interest (GOI) but also one or more coding sequences for certain RNAi, antisense, sgRNA, miRNA or inhibitors thereof. A representative, non-limiting configuration of the recombinant viral vector of the invention is illustrated in FIG. 1. For example, the recombinant viral vector of the invention may be a divergent AAV vector, such as AAV9 vector, designed to express a version of a functional dystrophin gene, such as any one of the μDys gene described herein above. The same divergent vector also expresses one or more additional coding sequence(s) from an independent/divergent transcription unit situated between the GOI promoter and the nearest ITR sequence (see FIG. 1). That is, at least one of such one or more additional coding sequence(s) is/are transcribed from an independent/divergent promoter different from the GOI promoter (such as the muscle specific CK8 promoter). The direction of transcription in the divergent transcription unit can be opposite to that of the GOI promoter. The design of the subject divergent vector permits independent and separate control of the GOI transcription unit and the divergent transcription unit, thus providing more flexibility and control in expression of the separate transcription units.

In case that the additional coding sequence encodes an miRNA, such as miR-29c, the backbone sequence of the miR-29c coding sequence can be modified such that the surrounding sequences for the mature miR-29c sequence are obtained from other miRNA, such as that for miR-30, -101, -155, or -451 (see above). It has been found that replacing the native surrounding sequences of miR-29c by those from miR-30, -101, -155, or -451 can enhance the production of the one strand (i.e., the guide strand) of miR-29c designed to target the miR-29c target sequence (i.e., reduce the production of its complement passenger strand that is not useful for targeting the miR-29c target sequence).

As controls, several so-called miR-29c “solo” expression constructs were generated on the same vector background. These miR-29c solo expressing constructs do not express μDys gene, but may instead express a reporter gene such as EGFP or GFP.

For example, one such solo vector may express the miR-29c coding sequence inserted into the intron sequence upstream of an EGFP coding sequence, all from an EF1A promoter. The backbone sequences of the miR-29c coding sequence may be modified by that of miR-30, -101, -155, or -451.

Another such solo vector may express an shRNA, such as shSLN that targets/down-regulates the expression of SLN. Expression of the shRNA may be driven by the U6 promoter that can be used by RNA Pol III, which produces strong transcription of short RNA transcripts. The shRNA coding sequence can be inserted into the intron in the U6 transcription cassette, before a coding sequence for GFP.

As a comparison, several so-called “fusion” vectors as described in International Patent Application No. PCT/US2019/065718, filed on Dec. 11, 2019 were also included in this experiment.

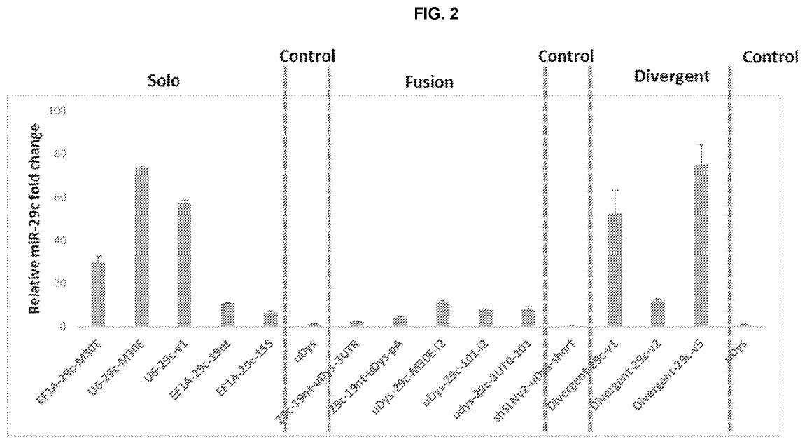

Specifically, several representative divergent, fusion, or solo vectors were used to transfect human iPS-derived cardiomyocytes in vitro, and the expression of miR-29c in the infected cardiomyocytes were determined, and the results were shown in FIG. 2.

Specifically, the five solo constructs, five fusion constructs, three divergent constructs, and a couple of control μDys expressing constructs were transfected to human iPS-derived cardiomyocytes according to standard procedure. Mature miR-29c levels were measured via Taqman stem-loop QPCR. The five solo constructs tested include U6- or EF1A-driven miR-29c expression cassettes designed in miR-30 (EF1A-29c-M30E and U6-29c-M30E) and miR-155 (EF1A-29c-19nt and EF1A-29c-155) backbones. The five fusion constructs tested include miR-29c expression cassettes designed in miR-101 (μDys-29c-101-i2 & μDys-29c-3UTR-101), miR-30 (μDys-29c-M30E-i2), and miR-155 (29c-19nt-μDys-3UTR & 29c-19nt-μDys-pa) backbones, inserted into intronic (i2), 3′UTR (3UTR) and after pA (pa) site locations relative to the μDys expression cassette. The three divergent constructs (Divergent-29c-v1, -v2, and -v5) all expressed miR-29c from the divergent expression cassette driven by a Pol III (U6) promoter.

It is apparent that the fusion constructs generally over-expressed miR-29c in the infected human iPS-derived cardiomyocytes by a factor of 2 to 11 fold, compared to a control in which a similar construct was used to express only μDys (and thus only background level of endogenous miR-29c expression was present).

Specific fusion constructs used to generate the data in FIG. 2 include:

29c-19nt-μDys-3UTR: a modified miR29c in miR-155 backbone, inserted into the 3′-UTR region of the μDys expression cassette (before the polyA adenylation signal sequence).

29c-19nt-μDys-pA: the same modified miR29c coding sequence in miR-155 backbone, inserted after the polyA adenylation signal sequence of the μDys expression cassette.

μDys-29c-M30E-i2: a modified miR29c coding sequence in miR-30E backbone, inserted into the intron region of the μDys expression cassette.

μDys-29c-101-i2: a modified miR29c coding sequence in miR-101 backbone, inserted into the intron region of the μDys expression cassette.

μDys-29c-3UTR-101: a modified miR29c coding sequence in miR-101 backbone, inserted into the 3′-UTR region of the μDys expression cassette.

Meanwhile, the solo constructs expressing miR-29c generally over-expressed miR-29c in the infected human iPS-derived cardiomyocytes by a factor of 6-73 fold, compared to the same control vector that expresses only μDys.

Specific solo constructs used to generate the data in FIG. 2 include:

EF1A-29c-M30E: a modified miR29c coding sequence in the miR-30E backbone, driven by the EF1A promoter.

U6-29c-M30E: a modified miR29c coding sequence in miR-30E backbone, driven by the Pol III U6 promoter.

U6-29c-v1: a miR29c coding sequence driven by the Pol III U6 promoter.

EF1A-29c-19nt: a modified miR29c coding sequence in miR-155 backbone, driven by the EF1A promoter.

EF1A-29c-155: another modified miR29c coding sequence in miR-155 backbone, driven by the EF1A promoter.

The three divergent constructs all expressed high to very high levels of miR-29c transcripts, with the v2 construct approaching that of the highest fusion construct μDys-29c-M30E-i2, while the v1 and v5 divergent constructs both approaching the highest solo constructs (50-70-fold). See FIG. 2.

Similar trends indicating (preferential) production of miR-29c from these constructs were also obtained when these constructs were evaluated in other in vitro cell systems, including the Mouly human healthy primary myoblasts, the mouse C2C12 immortalized myoblast line, and mouse fibroblast NIH3T3 cells, all without changes in μDys expression from the same vector (data not shown). Thus, insertion of the miR-29c expression cassette into the subject divergent vectors which also contain a μDys expression cassette does not cause significant reductions (if any) in μDys mRNA production.

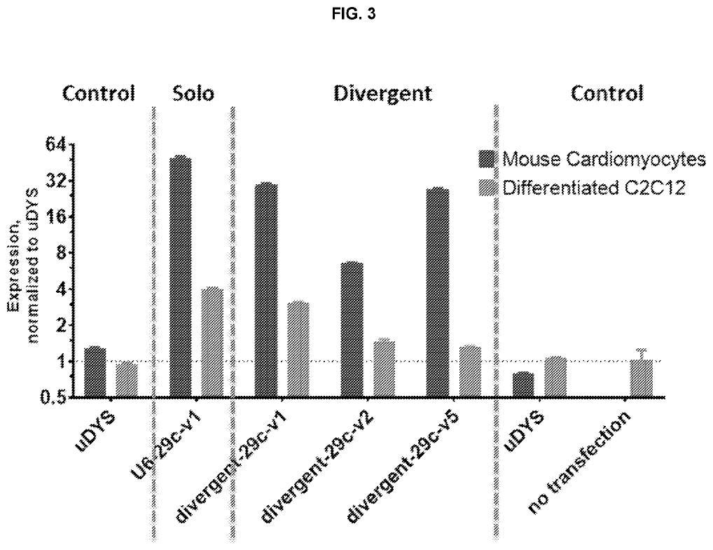

A few selected divergent recombinant viral vectors in AAV9 viral particles (Divergent-29c-v1, -v2, and -v5) were also used to infect differentiated C2C12 myotube and primary mouse cardiomyocytes, and expression of miR-29c was confirmed in these cells as well. See FIG. 3, with results being expressed as relative miR-29c expression after normalization against controls expressing only μDys.

In this experiment, μDys production appeared largely unaffected relative to control group. In addition, miR-29c passenger strand levels did not show increased levels.

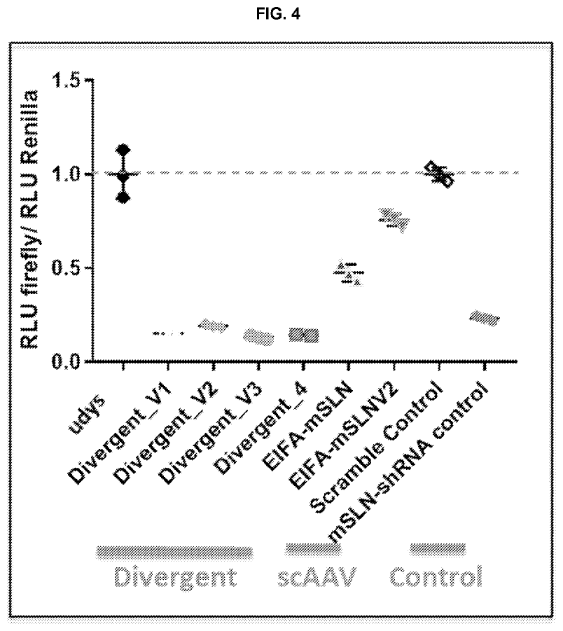

Meanwhile, expression of shmSLN from the subject divergent constructs, and the resulting ˜90% down-regulation of mouse SLN-firefly construct levels in mouse C2C12 cells transfected by such divergent constructs, were shown in FIG. 4.

The various constructs used in FIG. 4 are described below.

μDys: control AAV9 vector encoding only the μDys (GOI).

EF1A-mSLN: a solo construct expressing only shRNA targeting mouse SLN (mSLN). Transcription of the shRNA coding sequence is driven by the EF1A promoter.

EF1A-mSLN (V2): another solo construct expressing only shRNA targeting mouse SLN. Transcription of the shRNA coding sequence is driven by the EF1A promoter.

mSLN-shRNA control: a commercial positive control shRNA targeting mouse SLN.

scramble: a commercial negative control shRNA with scrambled sequence of the positive control shRNA.

The four divergent constructs (Divergent_V1 to _V4) were described above.

In contrast to the fusion constructs that achieved about 50% mSLN expression knock down (data not shown), the subject divergent constructs consistently achieved >90% mSLN knock down in a dual luciferase based assay, with the results being expressed as the ratio of RLU (relative luciferase unit) from the firefly luciferase to RLU from the renilla luciferase. The results in FIG. 4 shows that all four tested divergent constructs (Divergent-V1, -V2, -V3 and 4) expressing shRNA targeting mSLN each knocked down mSLN expression by >90%, compared to a control construct expressing only μDys but no shRNA against mSLN (μDys) (with a normalized ratio of 1.0). In comparison, the EF1A-mSLN and EF1A-mSLNV2 fusion constructs knocked down mSLN by about 50% to 30%, respectively, in similar dual luciferase-based assays. The mSLN-shRNA positive control similarly knocked down about 80-90% mSLN expression, while the scrambled control had no effect. See FIG. 4.

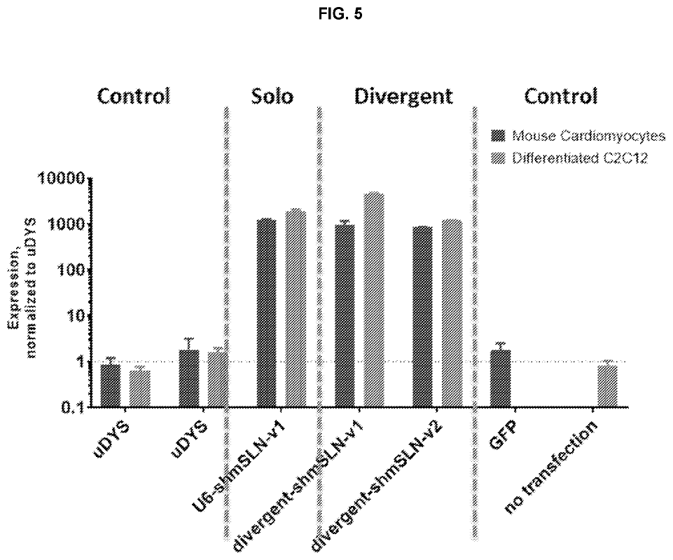

FIG. 5 shows relative expression levels of siSLN (processed siRNA product from the transcribed shSLN) in differentiated C2C12 myotubes or mouse primary cardiomyocytes for the various recombinant AAV9 vectors encoding shmSLN, either as the sole coding sequence in the viral vector (“Solo”), or as part of the divergent construct of the present disclosure (“Divergent”). siRNA production was quantified via a custom Taqman stem-loop QPCR system. The relative siSLN expression levels of the solo and divergent constructs were normalized against the level in the μDys control group, although apparent high fold changes may not be informative due to the near absent or very minimal siSLN-like RNA production in the control group. Nevertheless, it is apparent that, in both cell types tested, the solo construct expressed about 1000-fold higher level of siSLN from the strong U6 Pol III promoter, as compared to the control group. Meanwhile, the tested divergent construct reached similarly high (if not higher) level of siSLN compared the solo constructs.

All the divergent AAV constructs have AAV yields that are largely comparable to that of the solo constructs expressing only μDys.

Numerous additional solo and divergent constructs expressing shRNA targeting human SLN were also tested in human iPS-derived cardiomyocytes. These include 6 solo constructs and 6 divergent constructs targeting human SLN. The results of these experiments were summarized in FIG. 6.

Specifically, several negative controls (e.g., multiple μDys and GFP plasmids) and positive controls were used in the experiments in FIG. 6. The negative controls include: two constructs (μDys1 and μDys2) expressing μDys alone (which had no effect on the expression level of SLN mRNA); a construct expressing GFP under the muscle-specific promoter CK8 (CK8-GFP) (which GFP also had no effect on SLN mRNA expression); and “sigma scramble”—a construct expressing a scrambled sequence of a hSLN-targeting shSLN (which expectedly had no effect on SLN mRNA expression). The positive control is “sigma shrna,” which is a commercially available shRNA plasmid from Sigma that encodes an hSLN-targeting shSLN that down-regulates about 80% of the hSLN mRNA.

Six solo constructs, each expressing a version of the shRNA targeting hSLN, and each under the transcriptional control of the strong Pol III U6 promoter, were tested and were shown to generally down-regulate about 80-90% of hSLN mRNA expression.

Up to 90-95% hSLN mRNA expression down-down were also observed across 6 solo constructs, and 4 divergent constructs of the invention. For example, the combo-c1-v1 construct is a divergent construct that co-expresses μDys and shRNA targeting hSLN. Up to 90% of the hSLN mRNA was knocked down upon infecting the human iPS-derived cardiomyocytes with this construct. The same was also observed for three other combo constructs, combo-c1-v2, combo-c2-v1, and combo-c2-v2.

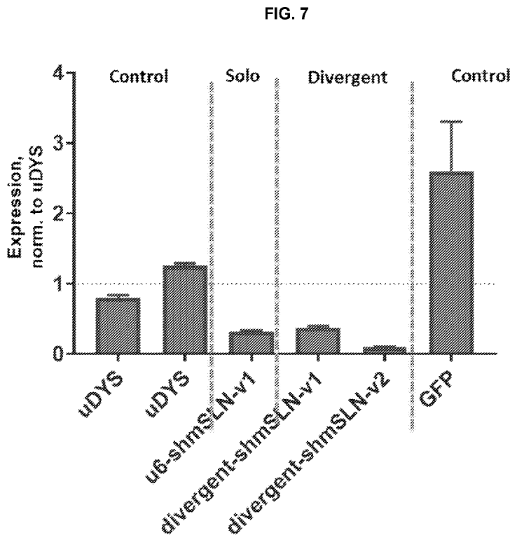

Similar results were also obtained in primary mouse cardiomyocytes, with up to 90% mSLN mRNA expression knock down using the solo or divergent AAV9 constructs of the invention expressing shRNA targeting mSLN. See FIG. 7.

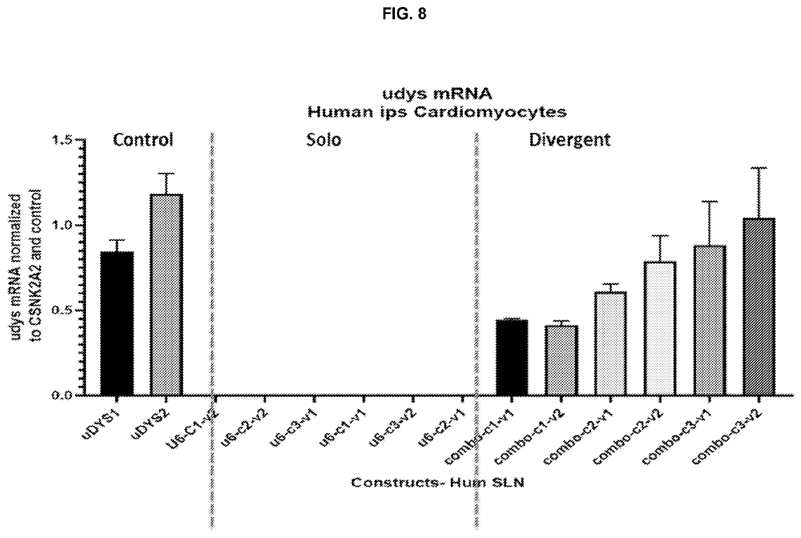

Although the divergent constructs greatly affected hSLN mRNA expression, they did not appear to have negative impact on the expression of the μDys from the same vector. As shown in FIG. 8, 6 solo and 6 divergent constructs targeting human SLN were transfected to human iPS-derived cardiomyocytes. Most divergent constructs showed largely similar (>50%) μDys mRNA expression as that of the control μDys-only constructs.

Denaturing agarose gel analysis of selected solo, fusion, and divergent constructs also confirmed that the AAV9 genomes of these miR-29c or shSLN constructs were largely intact. See FIG. 9. Furthermore, the ratio of all three AAV9 capsid proteins VP1-VP3 remains the same across all AAV9-based solo, fusion, and divergent vectors in FIG. 9. See FIG. 10.

These results demonstrate that AAV9 viral vectors produced from the subject divergent vectors, like the fusion vectors, have genome integrity.

### Example 2 In Vivo Expression of Coding Sequences from Divergent Constructs

This experiment demonstrates that the subject divergent constructs, like the fusion constructs, can be used to simultaneously express μDys and one or more additional coding sequence(s) that affect a separate pathway (e.g., down-regulation of SLN, and/or up-regulation of miR-29c) to achieve better-than-solo if not synergistic therapeutic efficacy.

In this set of experiments, several fusion and divergent constructs of AAV9 encoding a μDys gene as well as a second coding sequence—either miR-29c or shmSLN targeting mouse SLN were used. These fusion and divergent constructs were injected into 6-weeks-old male mdx mice via tail vein, at a dose of about 5E13 vg/kg (except for one group, U6-29c-v1, at 1E14 vg/kg). Expression of μDys, miR-29c, and SLN mRNA were then monitored over a period of 28 days post injection. The detailed experimental set-ups are summarized below:

| Group | Type | Name | Animal Number |
|---|---|---|---|
| miR | μDys | μDys | 4 |
| miR | Solo | U6-29c-v1 | 4 |
| miR | Fusion | μDys-29c-M30E-i2 | 4 |
| miR | Fusion | μDys-29c-101-3UTR | 4 |
| miR | Divergent | Divergent-29c-v1 | 3 |
| miR | Divergent | Divergent-29c-v2 | 2 |
| miR | Divergent | Divergent-29c-v5 | 4 |
| miR | Solo-1E14 | U6-29c0v1 (2×) | 2 |
| miR | Control | Control | 4 |
| shSLN | μDys | μDys | 4 |
| shSLN | Solo | U6-shmSLN-v1 | 4 |
| shSLN | Fusion | μDys-shmSLNv2 | 4 |
| shSLN | Divergent | Divergent-shmSLN-v1 | 4 |
| shSLN | Divergent | Divergent-shmSLN-v2 | 2 |

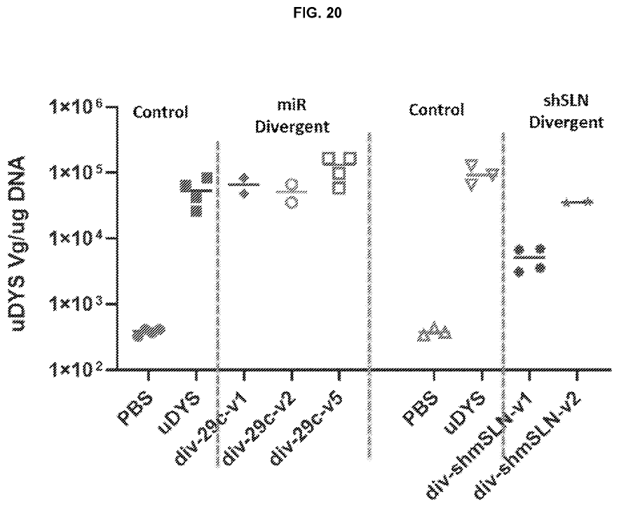

In the miR-29c experimental group, it was found that the two tested fusion constructs, one in M30E backbone and inserted into the intron of the μDys expression cassette, and one in miR-101 backbone and inserted into the 3′-UTR region of the μDys expression cassette, led to 1.4-2.8-fold miR-29c up-regulation in left gastrocnemius (see FIG. 20A of PCT/US2019/065718), diaphragm (see FIG. 20B of PCT/US2019/065718), and left ventricle (see FIG. 20C of PCT/US2019/065718). The miR-29c-μDys fusion AAV9 constructs were administered at 5E13 vg/kg dose. The solo U6 promoter-driven miR-29c construct in AAV9 produced 2-11 fold up-regulation at 5E13 vg/kg dose, and 6-16-fold at 1E14 vg/kg dose.

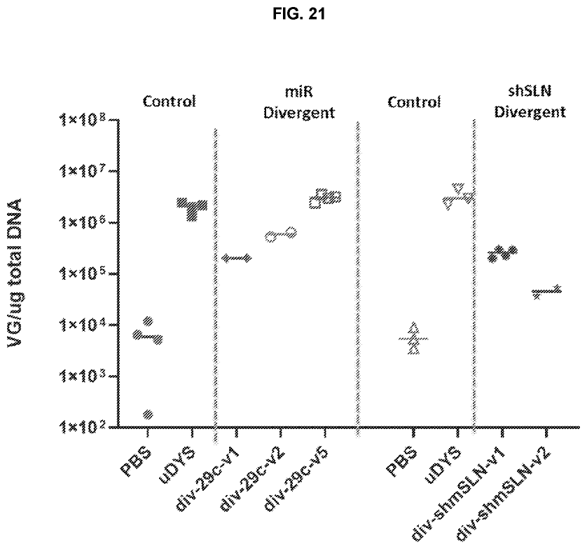

Meanwhile, miR-29c up-regulation by the fusion AAV9 constructs did not result in reduction of μDys production in gastrocnemius (see FIG. 21 of PCT/US2019/065718), diaphragm (data not shown) and left ventricle (data not shown). The fusion AAV9 constructs showed similar μDys expression, at both RNA and protein levels, as that of the control μDys-only AAV9 constructs. Solo constructs expressing only miR-29c do not produce μDys, therefore showed absent μDys levels.

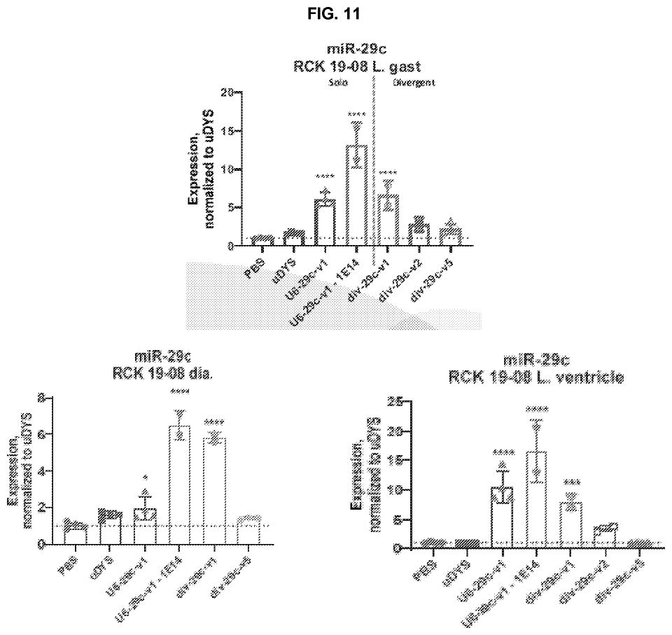

Similarly, it was found that the three tested divergent constructs (Divergent-29c-v1, -v2, and -v5) led to 2-6-fold miR-29c up-regulation in left gastrocnemius (FIG. 11, top panel), up to 5.8-fold miR-29c up-regulation in diaphragm (FIG. 11, lower left panel), and up to 7.5-fold miR-29c up-regulation in left ventricle (FIG. 11, lower right panel).

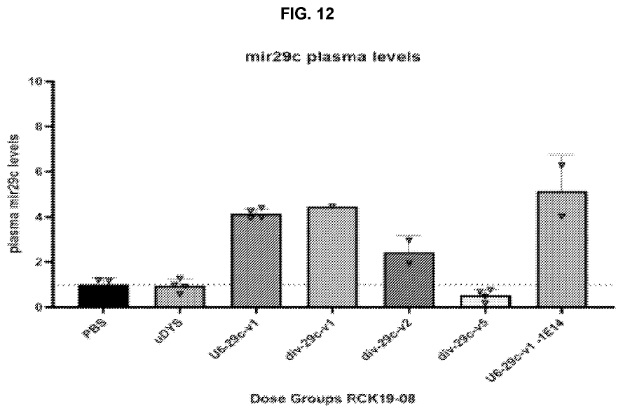

Interestingly, increased level of miR-29c expression can also be detected in the plasma (FIG. 12), suggesting that serum/plasma level of miR-29c may be used as a biomarker to track miR-29c expression level.

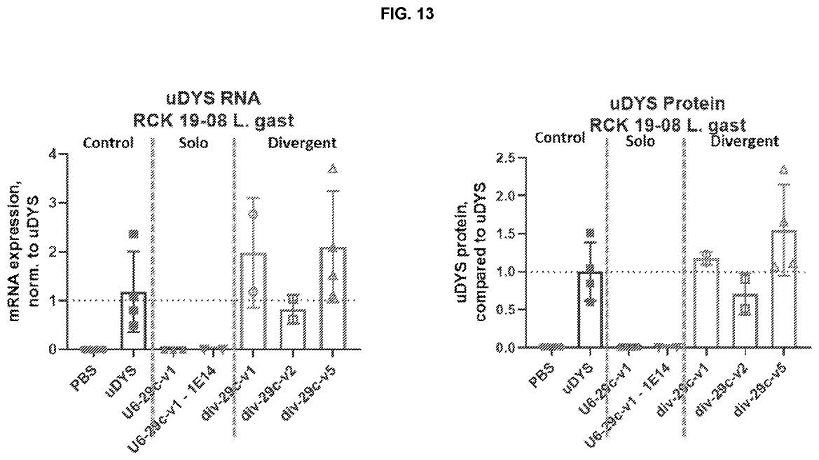

miR-29c up-regulation by the divergent AAV9 constructs also did not result in reduction of μDys production in the left gastrocnemius (FIG. 13, left panel for RNA, right panel for protein), diaphragm (data not shown) and left ventricle (data not shown). The divergent AAV9 constructs showed similar μDys expression, at both RNA and protein levels, as that of the control μDys-only AAV9 constructs. Solo constructs expressing only miR-29c do not produce μDys, therefore showed negligible μDys levels.

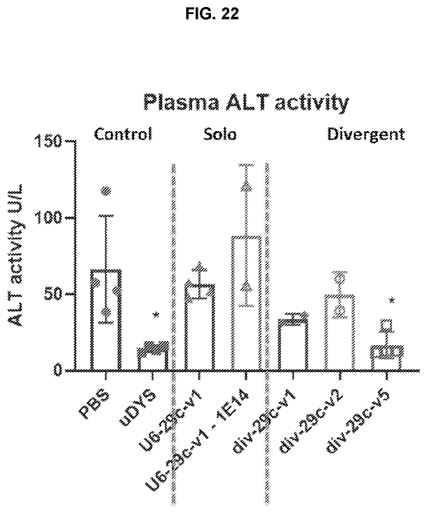

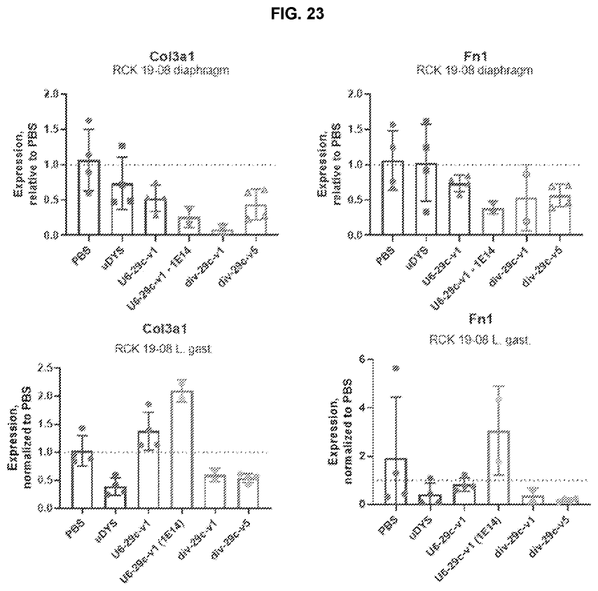

In the shmSLN experimental group, it was found that the tested shmSLN fusion AAV9 construct led to up to 50% mSLN mRNA down-regulation in the diaphragm, left gas, and atrium (see FIG. 22 of PCT/US2019/065718), as well as in tongue (data not shown). Similarly, mSLN mRNA down-regulation by the fusion AAV9 construct did not result in reduction of μDys production, at both RNA and protein levels, in gastrocnemius (see FIG. 23 of PCT/US2019/065718), diaphragm (data not shown), and left ventricle (data not shown), as compared to that of the control AAV9 expressing only μDys. Solo construct expressing only shmSLN did not produce μDys, thus showing absent μDys levels. Diaphragm results are shown. Similar results in tongue and atrium.

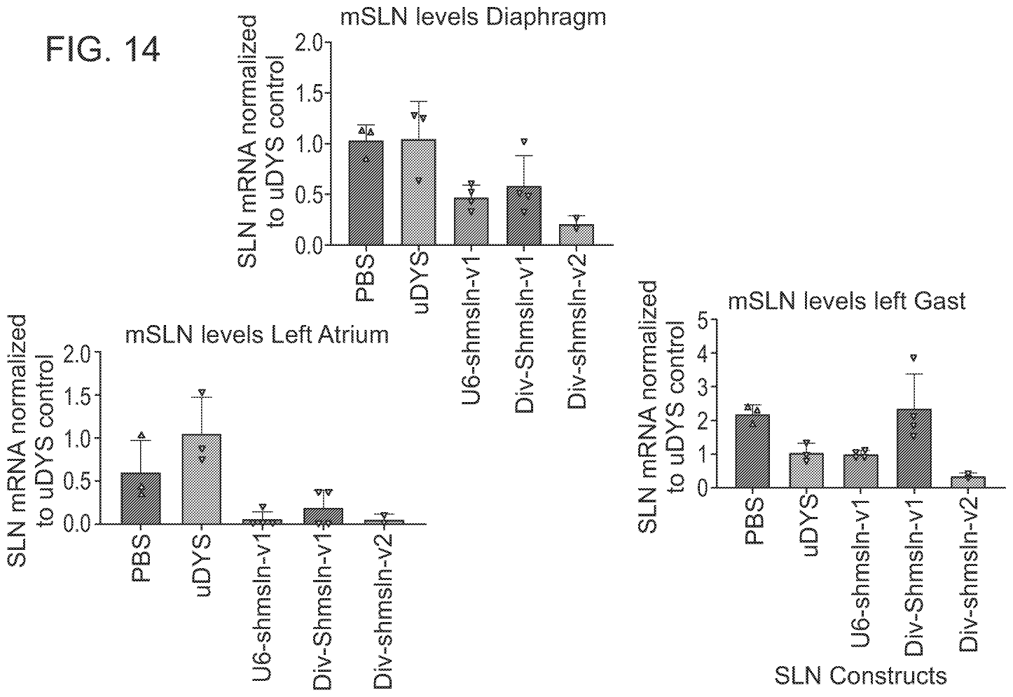

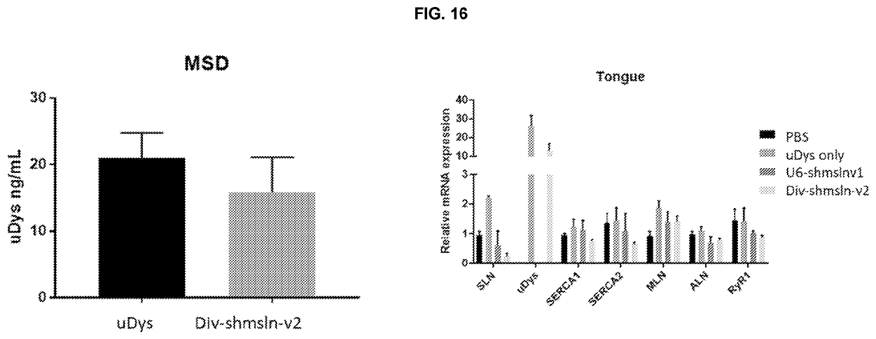

Similarly, it was found that the two tested shmSLN divergent AAV9 constructs (Divergent-shmSLN-v1 and -v2) led to up to 75% mSLN mRNA down-regulation in the diaphragm (FIG. 14, top panel), up to 95% mSLN mRNA down-regulation in the left atrium (FIG. 14, lower left panel), and up to 80% mSLN mRNA down-regulation in the left gast (FIG. 14, lower right panel), as well as in tongue (see FIG. 16). siRNA productions were separately confirmed in these experiments.

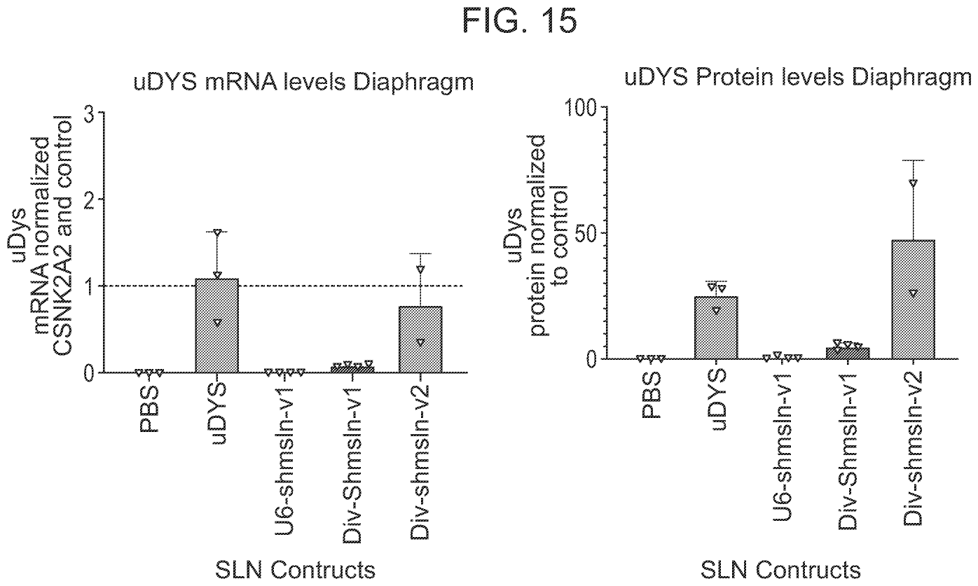

mSLN mRNA down-regulation by one divergent AAV9 construct also did not result in reduction of μDys production, at both RNA and protein levels, in diaphragm (FIG. 15, left panel for RNA, right panel for protein), tongue (FIG. 16, left panel), and atrium (data not shown), as compared to that of the control AAV9 expressing only μDys, except that the Divergent-shmSLN-v2 construct reduced μDys expression in the left gastrocnemius by 60-70%. Solo construct expressing only shmSLN did not produce μDys, thus showing absent μDys levels.

These data show that the subject divergent constructs can simultaneously express both the μDys gene and at least one additional coding sequence such as miR-29c or shRNA against SLN, thus achieving better therapeutic outcome compared to viral vectors expressing only one coding sequence such as μDys.

### Example 3 Coding Sequences Expressed In Vivo from the Divergent Constructs are Biologically Active

This experiment demonstrates that the coding sequences expressed from the divergent constructs of the invention are biologically active.

Dystrophin provides structural stability to the muscle cell membrane, and increased permeability of the sarcolemma leads to the release of creatine kinase (CK) from muscle fibers. Thus, increased creatine kinase (CK) levels are a hallmark of muscle damage. In DMD patients, CK levels are significantly increased above the normal range (e.g., 10-100 times the normal level since birth). Likewise, serum CK levels are considered as a general measure of muscle health in the mdx mouse model.

The data in this experiment shows that miR-29c solo (administered at the high dose of 1E14 vg/kg) and miR-29c-μDys divergent (administered at 5E13 vg/kg dose) constructs of AAV9 both reduced serum CK levels in the mdx mouse model, to the similar extent compared to the μDys control, therefore suggesting a therapeutic benefit of expressing miR-29c in DMD patients.

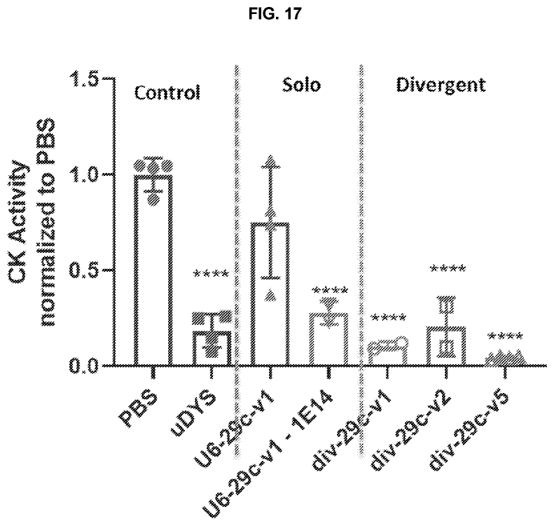

Specifically, in the in vivo experiments of Example 2, serum CK levels were also determined for the various groups of mice. FIG. 17 shows that expression of μDys alone caused significant drop in serum CK level. Co-expressing μDys and miR-29c, with all three tested divergent constructs, also led to similarly significant drop in serum CK levels. Interestingly, expressing miR-29c alone also led to significant decrease of serum CK level, especially when a higher viral dose (of miR-29c-expressing solo constructs) was used.

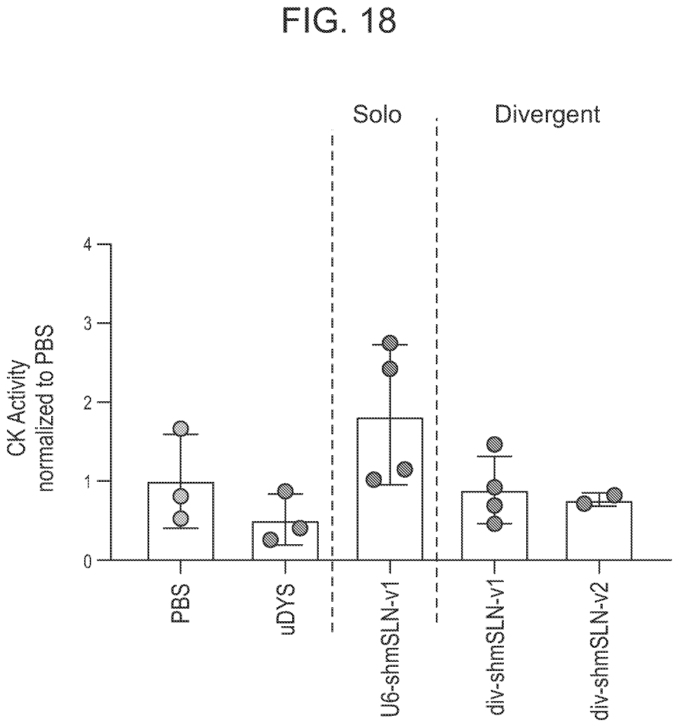

In FIG. 18, expression of shmSLN from either the solo or divergent constructs did not appear to reduce serum CK level.

On the other hand, tissue inhibitors of metalloproteinase-1 (TIMP-1) has been proposed as a serum biomarker for monitoring disease progression and/or treatment effects in Duchenne muscular dystrophy (DMD) patients, since serum levels of TIMP-1 were significantly higher in DMD patients compared to healthy controls. Similarly, TIMP1 is also a serum marker for muscle health in the mdx mouse model.

Thus in the in vivo experiments of Example 2, serum TIMP1 levels were also determined for the various groups of mdx mice. It was shown in FIG. 25, left panel of PCT/US2019/065718 that expression of μDys alone caused significant drop in serum TIMP1 level. Co-expressing μDys and miR-29c, with both tested fusion constructs, also led to similarly significant drop in serum TIMP1 levels. Meanwhile, expressing miR-29c alone did not lead to decrease of serum TIMP1 level, even when a higher viral dose (of miR-29c-expressing solo constructs) was used.

Likewise, FIG. 25 of PCT/US2019/065718, right panel, shows that expression of μDys alone caused significant drop in serum TIMP1 level. Co-expressing μDys and shRNA against mSLN with the tested fusion construct also led to similarly significant drop in serum TIMP1 levels. Meanwhile, expressing shRNA against mSLN alone did not lead to decrease of serum TIMP1 level.

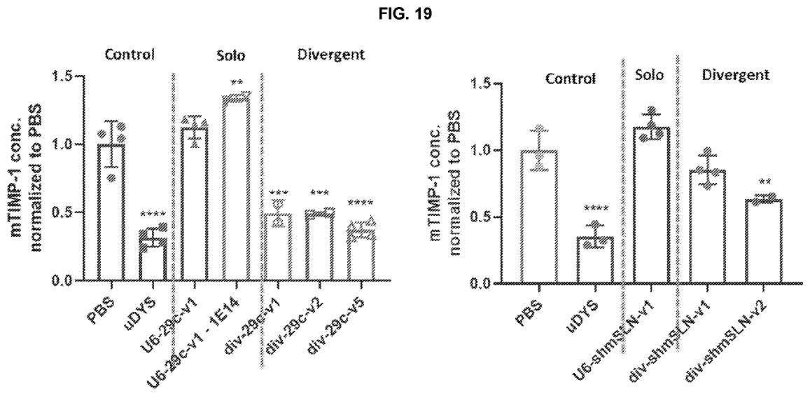

Here, similar results were obtained from the divergent constructs. In FIG. 19, left panel, expression of μDys alone caused significant drop in serum TIMP1 level. Co-expressing μDys and miR-29c, with all three tested divergent constructs, also led to similarly significant drop in serum TIMP1 levels. Meanwhile, expressing miR-29c alone did not lead to decrease of serum TIMP1 level, even when a higher viral dose (of miR-29c-expressing solo constructs) was used.

Likewise, in FIG. 19, right panel, expression of μDys alone caused significant drop in serum TIMP1 level. Co-expressing μDys and shRNA against mSLN with the tested divergent construct also led to similarly significant drop in serum TIMP1 levels. Meanwhile, expressing shRNA against mSLN alone in the solo construct did not lead to decrease of serum TIMP1 level.

### Example 4 the Divergent Constructs Show Comparable Biodistribution Compared to Control Constructs in Gastrocnemius

In the in vivo experiments in Example 2, biodistribution of the divergent viral vectors was compared to that of the solo viral vector expressing only μDys. It was found that biodistribution for most viral vectors used were largely similar in gastrocnemius, regardless of whether the divergent construct expresses miR-29c or shmSLN. See FIG. 20. One of the divergent vector encoding shmSLN, however, appeared to be lower compared to that of the μDys solo construct.

### Example 5 Two Divergent Constructs Showed Reduced Biodistribution in the Liver

In the in vivo experiments in Example 2, liver levels of the divergent viral vectors were compared to that of the solo viral vector expressing only μDys. It was found that viral titers of most viral vectors used were somewhat lower in liver, regardless of whether the divergent construct expresses miR-29c or shSLN. See FIG. 21. The only exception seems to be the DIV-29c-v5 vector expressing miR-29c, which apparently had the same (if not higher) viral titer compared to that of the μDys solo construct.

In order to determine whether liver damage was the cause for the apparent lower titer in the liver, plasma ALT levels were assessed for the various groups infected by the divergent vectors. The results in FIG. 22 showed that liver damage is unlikely to be the cause of lower titer in liver, because the two divergent constructs having lower viral titers in the liver, DIV-29c-v1 and -v2, both had plasma ALT levels comparable, if not lower than that of the PBS control.

### Example 6 Enhanced Therapeutic Efficacy Using the Divergent Constructs Compared to μDys Single Therapy

In order to determine whether co-expressing μDys and miR-29c leads to better therapeutic efficacy and/or less complication such as fibrosis, the expression levels of two fibrotic marker genes, Col3a1 and Fn1, were examined in mice administered with the various divergent, solo, or control constructs in Example 2. Col3A1 expression and FN1 expression have been used as markers of fibrotic activity.

In the diaphragm, solo AAV9 vectors expressing only μDys or miR-29c resulted in about 35-50% decreased expression of the fibrosis marker gene Col3A1. Higher dose of the solo miR-29c construct (at 1E14 vg/kg) reduced Col3A1 expression further to about ⅓ of the control level (FIG. 23, upper left panel). One of the divergent construct, DIV-29c-v1 dramatically reduced Col3A1 level by over 90%, which was unexpected given the level of reduction by μDys and miR-29c alone. The other divergent vector, DIV-29c-v5, reduced Col3A1 expression to the same extent of the miR-29c solo construct U6-29c-v1.

Expression of the other fibrosis gene Fn1 was also reduced, though to a lesser extent. While the μDys alone construct did not seem to significantly reduce Fn1 expression in the diaphragm, the miR-29c solo construct did moderately reduce Fn1 expression by about 25% at the normal dose of 5E13 vg/kg, and by more than 50% at the high dose of 1E14 vg/kg. Both divergent vectors reduced Fn1 expression at a level between the normal and high titer miR-29c.

In left gastrocnemius, however, expression level of Col3A1 was reduced by μDys by more than 50%, but was apparently increased by miR-29c by about 50% at normal titer, and 100% at high titer. Both divergent vectors reduced Col3A1 expression in the left gast to about just under 50%.

Similar results were observed for Fn1 expression in left gastrocnemius. While μDys reduced Fn1 expression and miR-29c increased Fn1 expression there, both divergent vectors unexpectedly reduced Fn1 expression to the same extent (if not better) than μDys alone.

It should be noted, however, that at this age of mdx mice (i.e., 10 weeks), fibrosis is typically only manifesting in diaphragm. Gastrocnemius is largely “normal” from fibrosis perspective.

These results show added benefit of the divergent constructs of the invention over μDys construct alone in diaphragm and possibly also in left gast, based on their effects on these two fibrotic marker genes.

### Example 7 Delivery of Enzyme-Based Gene Editing: CRISPR/Cas and sgRNA/crRNA

The subject viral vectors, e.g., rAAV viral vectors can be used to deliver CRISPR/Cas9 or CRISPR/Cas12a (or other engineered or modified Cas enzymes or homologous thereof) into a target cell, together with one or more sgRNA (for Cas9), or one or more crRNA (for Cas12a), for simultaneous knock down of target genes in the target cell. The target cell tropism can be controlled in part by the tropism of the viral particles in which the CRISPR/Cas and sgRNA/crRNA-encoding sequences resides.

For example, for AAV-mediated delivery, the GOI in the subject viral vector can be the coding sequence for CRISPR/Cas9 or CRISPR/Cas12a. The one or more sgRNA or crRNA that can be loaded onto Cas9 or Cas12a, respectively, can be expressed from the divergent expression cassette, the intron, the 3′-UTR, and/or elsewhere in the expression cassette of Cas9/Cas12a.

Upon infecting the target cell with the subject viral vectors, e.g., AAV vectors, Cas proteins and sgRNA/crRNA are co-expressed inside the target cell to mediate gene editing.

1. A recombinant viral vector comprising:
a) a first transcription cassette for expressing a first gene of interest (1st GOI) under the control of an operably linked first control element;
b) a second transcription cassette for expressing a second gene of interest (2nd GOI) under the control of an operably linked second control element;
wherein said first transcription cassette and said second transcription cassette do not overlap in sequence, and,
wherein said first control element and said second control element transcribes the 1st GOI and the 2nd GOI, respectively, in opposite directions away from each other.

2. The recombinant viral vector of claim 1, wherein:
said first gene of interest encodes a wild-type or normal gene (e.g., codon optimized wild-type or normal gene) that is defective in a disease or condition, and wherein said second gene of interest encodes an antagonist that targets a product of said gene defective in the disease or condition; or,
wherein said first gene of interest encodes a CRISPR/Cas enzyme (e.g., Cas9, Cas12a, Cas13a-13d), and wherein said second gene of interest encodes one or more guide RNA (e.g., sgRNA for Cas9, or crRNA for Cas12a) each specific for a target sequence; or
wherein said first gene of interest and said second gene of interest encode products that function in distinct pathways beneficial in the treatment of a disease or condition.

3-4. (canceled)

5. The recombinant viral vector of claim 1, wherein:
a) said first GOI comprises a heterologous intron sequence that enhances expression of a downstream protein-coding sequence, a 3′-UTR coding region downstream of the protein-coding sequence, and the polyadenylation (polyA) signal sequence (e.g., AATAAA);
b) said second GOI comprises one or more coding sequences that independently encode: a protein, a polypeptide, an RNAi sequence (siRNA, shRNA, miRNA), an antisense sequence, a guide sequence for a gene editing enzyme, a microRNA (miRNA), and/or a miRNA inhibitor; and,
c) optionally, one or more additional coding sequences inserted in the heterologous intron sequence and/or in the 3′-UTR coding region of the first GOI, wherein said one or more additional coding sequences independently encode: a protein, a polypeptide, an RNAi sequence (siRNA, shRNA, miRNA), an antisense sequence, a guide sequence for a gene editing enzyme, a microRNA (miRNA), and/or a miRNA inhibitor.

6. The recombinant viral vector of claim 1, wherein the recombinant viral vector is a recombinant AAV (adeno associated viral) vector or a lentiviral vector.

7. (canceled)

8. The recombinant viral vector of claim 1, wherein expression of the first GOI and/or the second GOI is substantially unaffected in the presence of each other.

9. The recombinant viral vector of claim 1, wherein:
the first GOI is a wt or normal SERPINA1 coding sequence (e.g., codon optimized SERPINA1 coding sequence), and wherein the second GOI encodes an RNAi agent (e.g., siRNA, shRNA, or miRNA) that targets a mutant allele of SERPINA1.

10-12. (canceled)

13. The recombinant viral vector of claim 1, wherein the first GOI is a wt or normal coding sequence for a gene defective in a repeat expansion disorder (RED) (e.g., a codon optimized wt or normal coding sequence for the gene defective in the RED), and wherein the second GOI encodes an RNAi agent (e.g., siRNA, shRNA, or miRNA) that targets a mutant allele of the gene defective in the RED.

14. The recombinant viral vector of claim 13, wherein:
the RED is spinocerebellar ataxia 3 (SCA3) resulting from a mutant ATXN3 gene with (more than 52) CAG trinucleotide repeats, and wherein the RNAi agent targets an SNP specifically associated with the mutant but not the wt allele of ATXN3; or,
wherein the RED is SCA1, 2, 3, 6, 7, 8, 10, 12, or 17, respectively, and wherein the RNAi agent targets an SNP specifically associated with the mutant but not the wt allele of ataxin-1, ataxin-2, ataxin-3, CACNA1, ataxin-7, SCA8, SCA10, PPP2R2B, or TBP, respectively; or, wherein the RED is myotonic dystrophy type 1 (DM1) resulting from a mutant DMPK gene with (more than 50) CTG trinucleotide repeats, and wherein the RNAi agent targets an SNP specifically associated with the mutant but not the wt allele of DMPKU; or
wherein the RED is myotonic dystrophy type 1 (DM1), wherein the first GOI encode a wt or codon-optimized MBNL1 gene, and wherein the second GOI encodes an RNAi agent (e.g., siRNA, shRNA, or miRNA) that targets a mutant allele of the DMPK gene defective in myotonic dystrophy type 1 (DM1) resulting from having more than 50 CTG trinucleotide repeats, or,
wherein the RED is Fragile X syndrome (FXS) resulting from a mutant FMR1 gene with (more than 55) CGG trinucleotide repeats, and wherein the RNAi agent targets an SNP specifically associated with the mutant but not the wt allele of FMR1.

15. The recombinant viral vector of claim 13, wherein: the RED is spinocerebellar ataxia 3 (SCA3) resulting from a mutant ATXN3 gene with (more than 52) CAG trinucleotide repeats, wherein the first GOI is a codon-optimized wt or normal coding sequence for ATXN3 having a 5′-UTR and/or a 3′-UTR different from that of the mutant ATXN3; and wherein the RNAi agent targets a 5′-UTR target sequence, a 3′-UTR target sequence, and/or a coding sequence target sequence specifically associated with the mutant but not the codon-optimized wt allele of ATXN3; or,
wherein the RED is SCA1, 2, 3, 6, 7, 8, 10, 12, or 17, respectively, wherein the first GOI is a codon-optimized wt or normal coding sequence for ataxin-1, ataxin-2, ataxin-3, CACNA1, ataxin-7, SCA8, SCA10, PPP2R2B, or TBP, respectively, having a 5′-UTR and/or a 3′-UTR different from that of the mutant ataxin-1, ataxin-2, ataxin-3, CACNA1, ataxin-7, SCA8, SCA10, PPP2R2B, or TBP, respectively; and wherein the RNAi agent targets a 5′-UTR target sequence, a 3′-UTR target sequence, and/or a coding sequence target sequence specifically associated with the mutant but not the codon-optimized wt allele of ataxin-1, ataxin-2, ataxin-3, CACNA1, ataxin-7, SCAB, SCA10, PPP2R2B, or TBP, respectively; or, wherein the RED is myotonic dystrophy type 1 (DM1) resulting from a mutant DMPK gene with (more than 50) CTG trinucleotide repeats, wherein the first GOI is a codon-optimized wt or normal coding sequence for DMPK having a 5′-UTR and/or a 3′-UTR different from that of the mutant DMPK; and wherein the RNAi agent targets a 5′-UTR target sequence, a 3′-UTR target sequence, and/or a coding sequence target sequence specifically associated with the mutant but not the codon-optimized wt allele of DMPK; or
wherein the RED is Fragile X syndrome (FXS) resulting from a mutant FMR1 gene with (more than 55) CGG trinucleotide repeats, wherein the first GOI is a codon-optimized wt or normal coding sequence for FMR1 having a 5′-UTR and/or a 3′-UTR different from that of the mutant FMR1; and wherein the RNAi agent targets a 5′-UTR target sequence, a 3′-UTR target sequence, and/or a coding sequence target sequence specifically associated with the mutant but not the codon-optimized wt allele of FMR1.

16. The recombinant viral vector of claim 14, wherein:
the first control element and/or the second control element comprises a neuron specific promoter and/or enhancer (such as the synapsin promoter), or a natural ATXN3 promoter;
the first control element and/or the second control element comprises a muscle specific promoter and/or enhancer (such as the CK8 promoter), or a natural DMPK promoter, or a ubiquitous promoter; or
the first control element and/or the second control element comprises a neuron specific promoter and/or enhancer (such as the synapsin promoter), or a natural FMR1 promoter.

17-25. (canceled)

26. The recombinant viral vector of claim 1, wherein the first GOI encodes a functional dystrophin protein (such as microD5) under the control of a muscle-specific promoter (such as the CK8 promoter);
optionally, wherein said second GOI encodes one or more coding sequences comprise an exon-skipping antisense sequence that induces skipping of an exon of a defective dystrophin, such as any one of exons 45-55 of dystrophin, or exon 44, 45, 51, and/or 53 of dystrophin.

27. (canceled)

28. The recombinant viral vector of claim 5, wherein said microRNA is miR-1, miR-133a, miR-29c, miR-30c, and/or miR-206; optionally, wherein said microRNA is miR-29c, optionally having a modified flanking backbone sequence that enhances the processing of the guide strand of miR-29c designed for a target sequence, and optionally, said modified flanking backbone sequence is from or based on miR-30, -101, -155, or -451.

29-30. (canceled)

31. The recombinant viral vector of claim 5, wherein said RNAi sequence is an shRNA against sarcolipin (shSLN).

32. The recombinant viral vector of claim 5, wherein said RNAi sequence (siRNA, shRNA, miRNA), said antisense sequence, said CRISPR/Cas9 sgRNA, said CRISPR/Cas12a crRNA and/or said microRNA antagonizes the function of one or more target genes, such as an inflammatory gene, an activator of NF-κB signaling pathway (e.g., TNF-α, IL-1, IL-1β, IL-6, Receptor activator of NF-κB (RANK), and Toll-like receptors (TLRs)), NF-κB, a downstream inflammatory cytokine induced by NF-κB, a histone deacetylase (e.g., HDAC2), TGF-β, connective tissue growth factor (CTGF), ollagens, elastin, a structural component of the extracellular matrix, Glucose-6-phosphate dehydrogenase (G6PD), myostatin, phosphodiesterase-5 (PED-5) or ACE, VEGF decoy-receptor type 1 (VEGFR-1 or Flt-1), and hematopoietic prostaglandin D synthase (HPGDS).

33. The recombinant viral vector of claim 5, wherein the heterologous intron sequence is SEQ ID NO: 1.

34. The recombinant viral vector of claim 1, wherein the vector is a recombinant AAV vector of the serotype AAV1, AAV2, AAV4, AAV5, AAV6, AAV7, AAVrh74, AAV8, AAV9, AAV10, AAV 11, AAV 12, or AAV 13.

35. A composition comprising the recombinant viral vector of claim 1.

36. The composition of claim 35, which is a pharmaceutical composition further comprising a therapeutically compatible carrier, diluent, or excipient.

37. The composition of claim 36, wherein:
the therapeutically acceptable carrier, diluent, or excipient is a sterile aqueous solution comprising 10 mM L-histidine at pH 6.0, 150 mM sodium chloride, and 1 mM magnesium chloride;
the composition is in a dosage form of about 10 mL of aqueous solution having at least 1.6×10¹³ vector genomes; and/or
the composition has a potency of at least 2×10¹² vector genomes per milliliter.

38-39. (canceled)

40. A method of producing the composition of claim 35, comprising producing the recombinant viral vector (e.g., the recombinant AAV vector) in a cell and lysing the cell to obtain the vector.

41. (canceled)

42. A method of treating Alpha-1 antitrypsin deficiency (AATD) in a subject in need thereof, the method comprising administering to the subject a therapeutically effective amount of the recombinant viral vector (e.g., the recombinant AAV vector) of claim 9, or the composition comprising the recombinant viral vector.

43. A method of treating spinocerebellar ataxia 3 (SCA3), myotonic dystrophy type 1 (DM1) or Fragile X syndrome (FXS) in a subject in need thereof, the method comprising administering to the subject a therapeutically effective amount of the recombinant viral vector (e.g., the recombinant AAV vector) of claim 14, or the composition comprising the recombinant viral vector.

44-45. (canceled)

46. The method of claim 43, wherein the recombinant AAV vector or the composition is administered by intramuscular injection, intravenous injection, parental administration or systemic administration.


SEQUENCE LISTING (61 sequences)

SEQ ID NO: 1 - DNA, 129 bp, Unknown
  Description of Unknown Heterologous intron sequence
gtatcaaggttacaagacaggtttaaggagaccaatagaaactgggcttgtcgagacagagaagactcttgcgtttctgataggcacctattggtcttactgacatccactttgcctttctctccacag

SEQ ID NO: 2 - DNA, 217 bp, Artificial Sequence
  Description of Artificial Sequence Synthetic polynucleotide
gtatcaaggttacaagacaggtttaaggagaccaatagaaactgggcttgtcgagacagatctcttacacaggctgaccgatttctcctggtgttcagagtctgtttttgtctagcaccatttgaaatcggttatgatgtagggggaagaagactcttgcgtttctgataggcacctattggtcttactgacatccactttgcctttctctccacag

SEQ ID NO: 3 - DNA, 88 bp, Unknown
  Description of Unknown miR-29c sequence
atctcttacacaggctgaccgatttctcctggtgttcagagtctgtttttgtctagcaccatttgaaatcggttatgatgtaggggga

SEQ ID NO: 4 - DNA, 722 bp, Homo sapiens
agacagcctgggaggggagaaggagttggagctcaagttggagacagcgaggagaaacctgccatagccagggtgtgtctttgatcctcttcaggaggtgaggagaagccagaggtccttggtgtgccctcagaaatctgcctgcagttctcaccaagccgctgtgaaaatggggataaacacccgggagctgtttctcaacttcactattgtcttgattacggttattcttatgtggctccttgtgaggtcctatcagtactgagaggccatgccatggtcctgggattgactgagatgctccggagctgcctgctctatgccctgagaccccactgctgtcattgtcacaggatgccattctccatccgagggcacctgtgacctgcactcacaatatctgctatgctgtagtgctaggattgattatgtgttctccaaagatgctgctcccaagggctgccaagtgtttgccagggaacggtagatttattccccaactcttaactgaaaatgtgttagacaagccacaaagttaaaattaaactggattcatgatgatgtaggattgttacaagcccctgatctgtctcaccacacatcccttcaacccacacggtctgcaaccaaactctaattcaacctgccagaaggaatgttagaggaagtctttgtcagcccttatagctatcatgtgaataaagttaagtcaacttcaaaaa

SEQ ID NO: 5 - protein, 31 aa, Homo sapiens
MGINTRELFLNFTIVLITVILMWLLVRSYGY

SEQ ID NO: 6 - DNA, 22 bp, Artificial Sequence
  Description of Artificial Sequence Synthetic oligonucleotide
accgatttcaaatggtgctaga

SEQ ID NO: 7 - DNA, 24 bp, Artificial Sequence
  Description of Artificial Sequence Synthetic oligonucleotide
tctagcaccatttgaaatcggtta

SEQ ID NO: 8 - DNA, 15 bp, Artificial Sequence
  Description of Artificial Sequence Synthetic oligonucleotide
gtgaagccacagatg

SEQ ID NO: 9 - DNA, 206 bp, Artificial Sequence
  Description of Artificial Sequence Synthetic polynucleotide
cagccactatgggtctaggctgcccatgtaaggaggcaaggcctggggacacccgagatgcctggttataattaacccagacatgtggctgctccccccccccaacacctgctgcctgagcctcacccccaccccggtgcctgggtcttaggctctgtacaccatggaggagaagctcgctctaaaaataaccctgtccctggtgg

SEQ ID NO: 10 - DNA, 358 bp, Artificial Sequence
  Description of Artificial Sequence Synthetic polynucleotide
gctgtgggggactgagggcaggctgtaacaggcttgggggccagggcttatacgtgcctgggactcccaaagtattactgttccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgcccccgggtcac

SEQ ID NO: 11 - DNA, 157 bp, Artificial Sequence
  Description of Artificial Sequence Synthetic polynucleotide
tcgacttcttaacccaacagaaggctcgagaaggtatattgctgttgacagtgagcgtaaccgatttcaaatggtgctatagtgaagccacagatgtatagcaccatttgaaatcggttatgcctactgcctcggacttcaaggggctagaattcga

SEQ ID NO: 12 - DNA, 159 bp, Artificial Sequence
  Description of Artificial Sequence Synthetic polynucleotide
tcgacttcttaacccaacagaaggctcgagaaggtatattgctgttgacagtgagcgaacttcactattgtcttgattactagtgaagccacagatgtagtaatcaagacaatagtgaagtttgcctactgcctcggacttcaaggggctagaattcga

SEQ ID NO: 13 - DNA, 159 bp, Artificial Sequence
  Description of Artificial Sequence Synthetic polynucleotide
tcgacttcttaacccaacagaaggctcgagaaggtatattgctgttgacagtgagcgaacacccgggagctgtttctcaatagtgaagccacagatgtattgagaaacagctcccgggtgtttgcctactgcctcggacttcaaggggctagaattcga

SEQ ID NO: 14 - DNA, 153 bp, Artificial Sequence
  Description of Artificial Sequence Synthetic polynucleotide
ggttaacccaacagaaggctaaagaaggtatattgctgttgacagtgagcgactagcaccatttgaaatcggttactgtgaagccacagatgggtaaccgattaaatggtgctagctgcctactgcctcggacttcaaggggctactttagga

SEQ ID NO: 15 - DNA, 204 bp, Artificial Sequence
  Description of Artificial Sequence Synthetic polynucleotide
ccaccagaaaggatgccgttgaccgacacagtgactgacaggctgccctggcgaaccgatttcaaatggtgcataccgtctattctaaaggtagcaccatttgaaatcggttaggatggcagccatcttaccttccatcagaggagcctcaccgtacccaggaagaaagaaggtgaaagaggaatgtgaaacaggtggctggga

SEQ ID NO: 16 - DNA, 133 bp, Artificial Sequence
  Description of Artificial Sequence Synthetic polynucleotide
cctggaggcttgctgaaggctgtatgctgtagcaccatttgaaatcggttattttggcctctgactgatgaccgctggaatggtgctacaggacacaaggcctgttactagcactcacatggaacaaatggcc

SEQ ID NO: 17 - DNA, 135 bp, Artificial Sequence
  Description of Artificial Sequence Synthetic polynucleotide
cctggaggcttgctgaaggctgtatgctgtagcaccatttgaaatcggttagttttggccactgactgactaaccgatcaaatggtgctacaggacacaaggcctgttactagcactcacatggaacaaatggcc

SEQ ID NO: 18 - DNA, 133 bp, Artificial Sequence
  Description of Artificial Sequence Synthetic polynucleotide
cctggaggcttgctgaaggctgtatgctggtgatgaggacaactgtgaaggttttggccactgactgaccttcacaggtcctcatcaccaggacacaaggcctgttactagcactcacatggaacaaatggcc

SEQ ID NO: 19 - DNA, 137 bp, Artificial Sequence
  Description of Artificial Sequence Synthetic polynucleotide
cctggaggcttgctgaaggctgtatgctggtgatgaggacaactgtgaagttgttttggccactgactgacaacttcacttgtcctcatcaccaggacacaaggcctgttactagcactcacatggaacaaatggcc

SEQ ID NO: 20 - DNA, 137 bp, Artificial Sequence
  Description of Artificial Sequence Synthetic polynucleotide
cctggaggcttgctgaaggctgtatgctggtaatcaagacaatagtgaagttgttttggccactgactgacaacttcacttgtcttgattaccaggacacaaggcctgttactagcactcacatggaacaaatggcc

SEQ ID NO: 21 - DNA, 139 bp, Artificial Sequence
  Description of Artificial Sequence Synthetic polynucleotide
cctggaggcttgctgaaggctgtatgctgttgagaaacagctcccgggtgttgttttggccactgactgacaacacccgggagctgtttctcaacaggacacaaggcctgttactagcactcacatggaacaaatggcc

SEQ ID NO: 22 - DNA, 215 bp, Artificial Sequence
  Description of Artificial Sequence Synthetic polynucleotide
ggacaggagagatgctgcaagcccaagaagctctctgctcagcctgtcacaacctactgactgccagggcacttgggaatggcaaggtagcaccatttgaaatcggttacgatttcaaatggtgctgtcttgctatacccagaaaacgtgccaggaagagaactcaggaccctgaagcagactactggaagggagactccagctcaaacaaggca

SEQ ID NO: 23 - DNA, 74 bp, Artificial Sequence
  Description of Artificial Sequence Synthetic oligonucleotide
gatcgtaaccgatttcaaatggtgctagccctgacccagctagcaccatttgaaatcggttattttttgaagct

SEQ ID NO: 24 - DNA, 65 bp, Artificial Sequence
  Description of Artificial Sequence Synthetic oligonucleotide
gatcgacttcacagttgtcctcatcactcgagtgatgaggacaactgtgaagcttttttgaagct

SEQ ID NO: 25 - DNA, 67 bp, Artificial Sequence
  Description of Artificial Sequence Synthetic oligonucleotide
gatcgacttcacagttgtcctcatcactcaagaggtgatgaggacaactgtgaagttttttgaagct

SEQ ID NO: 26 - DNA, 63 bp, Artificial Sequence
  Description of Artificial Sequence Synthetic oligonucleotide
taaccgatttcaaatggtgctagccctgacccagctagcaccatttgaaatcggttatttttt

SEQ ID NO: 27 - DNA, 57 bp, Artificial Sequence
  Description of Artificial Sequence Synthetic oligonucleotide
taaccgatttcaaatggtgctatcaagagtagcaccatttgaaatcggttatttttt

SEQ ID NO: 28 - DNA, 62 bp, Artificial Sequence
  Description of Artificial Sequence Synthetic oligonucleotide
taaccgatttcaaatggtgctacttcctgtcagatagcaccatttgaaatcggttatttttt

SEQ ID NO: 29 - DNA, 59 bp, Artificial Sequence
  Description of Artificial Sequence Synthetic oligonucleotide
taaccgatttcaaatggtgctattcaagagatagcaccatttgaaatcggttatttttt

SEQ ID NO: 30 - DNA, 141 bp, Artificial Sequence
  Description of Artificial Sequence Synthetic polynucleotide
cctggaggcttgctgaaggctgtatgctgtagcaccatttgaaatcggttagttttggccactgactgactaaccgatcaaatggtgctacaggacacaaggcctgttactagcactcacatggaacaaatggcctttttt

SEQ ID NO: 31 - DNA, 54 bp, Artificial Sequence
  Description of Artificial Sequence Synthetic oligonucleotide
acttcacagttgtcctcatcactcgagtgatgaggacaactgtgaagctttttt

SEQ ID NO: 32 - DNA, 53 bp, Artificial Sequence
  Description of Artificial Sequence Synthetic oligonucleotide
acttcacagttgtcctcatcactcgagtgatgaggacaactgtgaagtttttt

SEQ ID NO: 33 - DNA, 62 bp, Artificial Sequence
  Description of Artificial Sequence Synthetic oligonucleotide
acttcacagttgtcctcatcacgccctgacccagcgtgatgaggacaactgtgaagtttttt

SEQ ID NO: 34 - DNA, 56 bp, Artificial Sequence
  Description of Artificial Sequence Synthetic oligonucleotide
acttcacagttgtcctcatcactcaagaggtgatgaggacaactgtgaagtttttt

SEQ ID NO: 35 - DNA, 52 bp, Artificial Sequence
  Description of Artificial Sequence Synthetic oligonucleotide
cttcactattgtcttgattactcgagtaatcaagacaatagtgaagtttttt

SEQ ID NO: 36 - DNA, 61 bp, Artificial Sequence
  Description of Artificial Sequence Synthetic oligonucleotide
cttcactattgtcttgattacgccctgacccagcgtaatcaagacaatagtgaagtttttt

SEQ ID NO: 37 - DNA, 52 bp, Artificial Sequence
  Description of Artificial Sequence Synthetic oligonucleotide
cacccgggagctgtttctcaatcgattgagaaacagctcccgggtgtttttt

SEQ ID NO: 38 - DNA, 61 bp, Artificial Sequence
  Description of Artificial Sequence Synthetic oligonucleotide
cacccgggagctgtttctcaagccctgacccagcttgagaaacagctcccgggtgtttttt

SEQ ID NO: 39 - DNA, 52 bp, Artificial Sequence
  Description of Artificial Sequence Synthetic oligonucleotide
tgtggctccttgtgaggtccttcgaaggacctcacaaggagccacatttttt

SEQ ID NO: 40 - DNA, 61 bp, Artificial Sequence
  Description of Artificial Sequence Synthetic oligonucleotide
tgtggctccttgtgaggtcctgccctgacccagcaggacctcacaaggagccacatttttt

SEQ ID NO: 41 - DNA, 1257 bp, Homo sapiens
atgccgtcttctgtctcgtggggcatcctcctgctggcaggcctgtgctgcctggtccctgtctccctggctgaggatccccagggagatgctgcccagaagacagatacatcccaccatgatcaggatcacccaaccttcaacaagatcacccccaacctggctgagttcgccttcagcctataccgccagctggcacaccagtccaacagcaccaatatcttcttctccccagtgagcatcgctacagcctttgcaatgctctccctggggaccaaggctgacactcacgatgaaatcctggagggcctgaatttcaacctcacggagattccggaggctcagatccatgaaggcttccaggaactcctccgtaccctcaaccagccagacagccagctccagctgaccaccggcaatggcctgttcctcagcgagggcctgaagctagtggataagtttttggaggatgttaaaaagttgtaccactcagaagccttcactgtcaacttcggggacaccgaagaggccaagaaacagatcaacgattacgtggagaagggtactcaagggaaaattgtggatttggtcaaggagcttgacagagacacagtttttgctctggtgaattacatcttctttaaaggcaaatgggagagaccctttgaagtcaaggacaccgaggaagaggacttccacgtggaccaggtgaccaccgtgaaggtgcctatgatgaagcgtttaggcatgtttaacatccagcactgtaagaagctgtccagctgggtgctgctgatgaaatacctgggcaatgccaccgccatcttcttcctgcctgatgaggggaaactacagcacctggaaaatgaactcacccacgatatcatcaccaagttcctggaaaatgaagacagaaggtctgccagcttacatttacccaaactgtccattactggaacctatgatctgaagagcgtcctgggtcaactgggcatcactaaggtcttcagcaatggggctgacctctccggggtcacagaggaggcacccctgaagctctccaaggccgtgcataaggctgtgctgaccatcgacgagaaagggactgaagctgctggggccatgtttttagaggccatacccatgtctatcccccccgaggtcaagttcaacaaaccctttgtcttcttaatgattgaacaaaataccaagtctcccctcttcatgggaaaagtggtgaatcccacccaaaaataa

SEQ ID NO: 42 - DNA, 1257 bp, Artificial Sequence
  Description of Artificial Sequence Synthetic polynucleotide
atgccaagtagtgtatcctggggaattctgctcttggctgggctctgttgccttgtcccagtgtctcttgccgaagaccctcagggtgacgcagctcagaaaaccgataccagtcatcacgatcaagatcaccctactttcaataaaataacgcccaaccttgcagaatttgcgttctctctgtatcggcagctcgcgcaccagtccaattcaaccaacatatttttctcaccggttagcatcgcaactgcgttcgcaatgttgtccctcggtacaaaagccgacacgcatgacgaaattttggaaggactgaactttaatctgaccgagataccggaggcccagattcacgagggatttcaagagcttcttcgcacactcaaccaaccggattctcagctgcagttgacaactgggaacggtctctttctgtctgagggacttaaacttgtagacaaatttcttgaggatgtcaagaagctctaccactccgaagcttttacggttaatttcggggatacggaagaggcgaaaaagcaaataaacgattatgtggaaaaaggaacacagggtaaaatcgttgatttggtaaaggagctggacagggacacagttttcgctctggtaaattacatattctttaagggcaaatgggagcgaccgttcgaagtcaaagacaccgaggaagaggactttcacgtggaccaagtgaccaccgtaaaggtccctatgatgaaacgcctcgggatgtttaatatccaacactgtaaaaaattgtccagctgggtcttgctgatgaagtatttgggaaatgcaaccgcaattttttttctgcctgatgaagggaagcttcagcatttggaaaacgagctgacacatgatataatcaccaaatttctggaaaatgaagacagaaggtccgctagtttgcatttgcctaaattgtccataacaggcacatacgatcttaagtccgtccttggacaactcggaatcacaaaggtcttcagtaacggtgccgatctgagtggagtcacagaagaagcccctttgaaattgtccaaagcggtacataaagcagtgcttactatagacgaaaagggcactgaagcagcaggggcgatgttcttggaggcgataccaatgtccattccaccagaggtcaagttcaacaagcccttcgtatttctcatgatcgaacagaatacaaaaagtcccctctttatgggtaaggtcgttaatcccacccaaaagtaa

SEQ ID NO: 43 - DNA, 66 bp, Artificial Sequence
  Description of Artificial Sequence Synthetic oligonucleotide
gatcgctgtgttcatggagcatctggtcaagagccagatgctccatgaacacagttttttgaagct

SEQ ID NO: 44 - DNA, 72 bp, Artificial Sequence
  Description of Artificial Sequence Synthetic oligonucleotide
gatcgctgtgttcatggagcatctgggccctgacccagcccagatgctccatgaacacagttttttgaagct

SEQ ID NO: 45 - DNA, 1086 bp, Homo sapiens
atggagtccatcttccacgagaaacaagaaggctcactttgtgctcaacattgcctgaataacttattgcaaggagaatattttagccctgtggaattatcctcaattgcacatcagctggatgaggaggagaggatgagaatggcagaaggaggagttactagtgaagattatcgcacgtttttacagcagccttctggaaatatggatgacagtggttttttctctattcaggttataagcaatgccttgaaagtttggggtttagaactaatcctgttcaacagtccagagtatcagaggctcaggatcgatcctataaatgaaagatcatttatatgcaattataaggaacactggtttacagttagaaaattaggaaaacagtggtttaacttgaattctctcttgacgggtccagaattaatatcagatacatatcttgcacttttcttggctcaattacaacaggaaggttattctatatttgtcgttaagggtgatctgccagattgcgaagctgaccaactcctgcagatgattagggtccaacagatgcatcgaccaaaacttattggagaagaattagcacaactaaaagagcaaagagtccataaaacagacctggaacgagtgttagaagcaaatgatggctcaggaatgttagacgaagatgaggaggatttgcagagggctctggcactaagtcgccaagaaattgacatggaagatgaggaagcagatctccgcagggctattcagctaagtatgcaaggtagttccagaaacatatctcaagatatgacacagacatcaggtacaaatcttacttcagaagagcttcggaagagacgagaagcctactttgaaaaacagcagcaaaagcagcaacagcagcagcagcagcagcagcagggggacctatcaggacagagttcacatccatgtgaaaggccagccaccagttcaggagcacttgggagtgatctaggtgatgctatgagtgaagaagacatgcttcaggcagctgtgaccatgtctttagaaactgtcagaaatgatttgaaaacagaaggaaaaaaataa

SEQ ID NO: 46 - DNA, 1086 bp, Artificial Sequence
  Description of Artificial Sequence Synthetic polynucleotide
atggaaagtatctttcatgaaaagcaagagggaagtttgtgcgctcaacattgtcttaacaacttgctgcaaggggagtatttcagtcctgtggagctctccagcatagcacaccaactcgatgaagaagagagaatgcggatggccgagggtggtgttacttcagaggattaccgcactttcttgcagcagcccagcgggaacatggacgactctggtttcttctctatccaggtcataagtaacgcgctcaaggtctggggtttggaactcattctcttcaactcccctgagtatcagagactgagaatagaccctataaacgagagatcctttatatgtaattacaaagaacattggtttactgtacggaaattgggcaagcagtggttcaatctgaactcattgctgacgggtcccgagctcatatccgacacgtatttggcgctttttctcgcccagttgcagcaggaaggttattccatttttgtagtaaagggagaccttccggattgtgaggcggatcagcttttgcagatgatccgggttcagcaaatgcatagaccgaagttgattggcgaggaacttgctcaactcaaagagcagcgcgtacacaaaacagacttggagcgggttctcgaagcaaatgatggaagcggaatgctggacgaagacgaggaagatctgcaacgagccctcgctttgagtcgacaagagatagacatggaagatgaagaggctgacttgagacgagcgatacagctgtccatgcagggaagttcccggaacatatctcaagacatgacgcaaacgagtggcacaaatctcactagtgaagagcttagaaagagacgcgaagcttacttcgagaagcaacagcagaaacaacaacaacagcagcaacagcaacaacagggtgatctcagcgggcaaagtagccacccgtgcgagaggcccgcaaccagcagtggggcgctggggtcagatctgggagatgcaatgtcagaagaagatatgttgcaggccgccgttactatgtcactcgaaacagtccgcaatgatctgaaaacagaaggtaaaaagtaa

SEQ ID NO: 47 - DNA, 66 bp, Artificial Sequence
  Description of Artificial Sequence Synthetic oligonucleotide
gatcgtgcgtcggttgtaggactaaatcaagagtttagtcctacaaccgacgcattttttgaagct

SEQ ID NO: 48 - DNA, 72 bp, Artificial Sequence
  Description of Artificial Sequence Synthetic oligonucleotide
gatcgtgcgtcggttgtaggactaaagccctgacccagctttagtcctacaaccgacgcattttttgaagct

SEQ ID NO: 49 - DNA, 140 bp, Artificial Sequence
  Description of Artificial Sequence Synthetic polynucleotide
aacagaaggctcgagaaggtatattgctgttgacagtgagcgtgcgtcggttgtaggactaaatagtgaagccacagatgtatttagtcctacaaccgacgcatgcctactgcctcggacttcaaggggctagaattcga

SEQ ID NO: 50 - DNA, 140 bp, Artificial Sequence
  Description of Artificial Sequence Synthetic polynucleotide
aacagaaggctcgagaaggtatattgctgttgacagtgagcgaatgcgtcggttgtaggactatagtgaagccacagatgtatagtcctacaaccgacgcatttgcctactgcctcggacttcaaggggctagaattcga

SEQ ID NO: 51 - DNA, 1908 bp, Homo sapiens
atgggagggcatttttggcccccagaaccttacacggtgtttatgtggggaagcccctgggaagcagacagtcctagggtgaagctgagaggcagagagaaggggagacagacagagggtggggctttcccccttgtctccagtgccctttctggtgaccctcggttcttttcccccaccacccccccagcggagcccatcgtggtgaggcttaaggaggtccgactgcagagggacgacttcgagattctgaaggtgatcggacgcggggcgttcagcgaggtagcggtagtgaagatgaagcagacgggccaggtgtatgccatgaagatcatgaacaagtgggacatgctgaagaggggcgaggtgtcgtgcttccgtgaggagagggacgtgttggtgaatggggaccggcggtggatcacgcagctgcacttcgccttccaggatgagaactacctgtacctggtcatggagtattacgtgggcggggacctgctgacactgctgagcaagtttggggagcggattccggccgagatggcgcgcttctacctggcggagattgtcatggccatagactcggtgcaccggcttggctacgtgcacagggacatcaaacccgacaacatcctgctggaccgctgtggccacatccgcctggccgacttcggctcttgcctcaagctgcgggcagatggaacggtgcggtcgctggtggctgtgggcaccccagactacctgtcccccgagatcctgcaggctgtgggcggtgggcctgggacaggcagctacgggcccgagtgtgactggtgggcgctgggtgtattcgcctatgaaatgttctatgggcagacgcccttctacgcggattccacggcggagacctatggcaagatcgtccactacaaggagcacctctctctgccgctggtggacgaaggggtccctgaggaggctcgagacttcattcagcggttgctgtgtcccccggagacacggctgggccggggtggagcaggcgacttccggacacatcccttcttctttggcctcgactgggatggtctccgggacagcgtgcccccctttacaccggatttcgaaggtgccaccgacacatgcaacttcgacttggtggaggacgggctcactgccatggagacactgtcggacattcgggaaggtgcgccgctaggggtccacctgccttttgtgggctactcctactcctgcatggccctcagggacagtgaggtcccaggccccacacccatggaactggaggccgagcagctgcttgagccacacgtgcaagcgcccagcctggagccctcggtgtccccacaggatgaaacagctgaagtggcagttccagcggctgtccctgcggcagaggctgaggccgaggtgacgctgcgggagctccaggaagccctggaggaggaggtgctcacccggcagagcctgagccgggagatggaggccatccgcacggacaaccagaacttcgccagtcaactacgcgaggcagaggctcggaaccgggacctagaggcacacgtccggcagttgcaggagcggatggagttgctgcaggcagagggagccacagctgtcacgggggtccccagtccccgggccacggatccaccttcccatatggccccccggccgtggctgtgggccagtgcccgctggtggggccaggccccatgcaccgccgccacctgctgctccctgccagggtccctaggcctggcctatcggaggcgctttccctgctcctgttcgccgttgttctgtctcgtgccgccgccctgggctgcattgggttggtggcccacgccggccaactcaccgcagtctggcgccgcccaggagccgcccgcgctccctgaaccctag

SEQ ID NO: 52 - DNA, 1908 bp, Artificial Sequence
  Description of Artificial Sequence Synthetic polynucleotide
atgggtgggcacttttggccgccggaaccctacacggtctttatgtggggctcaccttgggaggcagacagtcctcgggtgaagttgcgcggccgagagaaaggcaggcaaacggaaggtggcgcttttcctttggtaagtagtgccctctctggggatcctagattcttttcaccgactacccctcccgcggagcctatagtagttagattgaaggaggttcgacttcaacgcgacgacttcgagatcctgaaggttataggtcggggggctttcagcgaggttgcggtggtaaagatgaagcagacaggtcaagtgtacgctatgaagattatgaacaagtgggacatgcttaaacggggagaagtctcatgtttcagggaggaaagagatgtactcgtaaacggagataggcggtggatcactcagctccacttcgcatttcaagatgaaaactacttgtacctggtaatggagtactatgtcggaggagaccttcttactcttttgtccaaattcggcgagcgcattccggcagaaatggcaagattctaccttgcagaaatagtgatggccatcgacagtgtacaccgactgggctacgtgcatagggacatcaaaccggacaacattcttcttgatcgctgcggccacatccggctggccgattttggctcctgcctcaagttgagggcggatggaaccgtccgaagtttggtggctgtaggaacaccagattacttgtctccagaaatattgcaagccgtcggaggtggccccgggaccggttcatacggtcccgagtgtgactggtgggcacttggcgtctttgcttacgaaatgttttatgggcaaactcccttctacgcagatagtactgcagaaacctatgggaagatagttcattataaggagcacctctcactcccattggtggatgaaggtgttcctgaggaagctcgcgatttcatacagcggttgctttgcccccctgagacgcggctgggacgagggggagcaggagacttccgcacccatcctttctttttcgggttggattgggacggtttgagagactcagtacctccattcacccccgacttcgaaggggcaacggatacatgcaactttgaccttgtcgaggacggactgactgcgatggaaacactgagtgatattcgcgaaggtgcgcccttgggtgtccacttgcccttcgttgggtactcatattcctgcatggcattgagggattccgaggtcccaggaccaacaccaatggagctcgaagccgagcagttgcttgaaccgcatgtgcaagcgcccagcctggagccgtctgttagtccccaggacgagactgccgaggtggcagtgcctgcagctgtcccggctgcagaagcagaggcagaagtcacgttgcgggagttgcaggaggcactcgaggaggaggtcctgacgcgccaatcacttagccgcgaaatggaagcaatccgcacggacaatcagaacttcgcgtcccaactgcgagaagcggaagcaagaaatcgagacttggaggcacacgtgcgccaattgcaagagcgcatggaactcctccaggcagaaggtgccacggctgtaaccggggttcccagtccaagagcaacagatcctccatctcacatggcaccacggccatggctgtgggcctccgcccggtggtgggggcaagcgccatgcacagcagctacttgttgttccttgccaggatctctcggtcttgcctatcgaaggcgcttcccttgtagttgctccccgctcttctgtcttgtcccaccgccttgggcagcactgggttggtggcctactccggcaaactccccacagtcaggtgcagcccaggaaccaccagccctcccagagccgtag

SEQ ID NO: 53 - DNA, 60 bp, Artificial Sequence
  Description of Artificial Sequence Synthetic oligonucleotide
  Description of Combined DNA/RNA Molecule Synthetic oligonucleotide
gatcggcugcugcugcugcugcutcaagagagcagcagcagcagcagcttttttgaagct

SEQ ID NO: 54 - DNA, 66 bp, Artificial Sequence
  Description of Artificial Sequence Synthetic oligonucleotide
  Description of Combined DNA/RNA Molecule Synthetic oligonucleotide
gatcggcugcugcugcugcugcugccctgacccagcagcagcagcagcagcagcttttttgaagct

SEQ ID NO: 55 - DNA, 1203 bp, Homo sapiens
atggctgttagtgtcacaccaattcgggacacaaaatggctaacactggaagtatgtagagagttccagagggggacttgctcacggccagacacggaatgtaaatttgcacatccttcgaaaagctgccaagttgaaaatggacgagtaatcgcctgctttgattcattgaaaggccgttgctccagggagaactgcaaatatcttcatccacccccacatttaaaaacgcagttggagataaatggacgcaataacttgattcagcagaagaacatggccatgttggcccagcaaatgcaactagccaatgccatgatgcctggtgccccattacaacccgtgccaatgttttcagttgcaccaagcttagccaccaatgcatcagcagccgcctttaatccctatctgggacctgtttctccaagcctggtcccggcagagatcttgccgactgcaccaatgttggttacagggaatccgggtgtccctgtacctgcagctgctgcagctgctgcacagaaattaatgcgaacagacagacttgaggtatgtcgagagtaccaacgtggcaattgcaaccgaggagaaaatgattgtcggtttgctcatcctgctgacagcacaatgattgacaccaatgacaacacagtcactgtgtgtatggattacatcaaagggagatgctctcgggaaaagtgcaaatactttcatccccctgcacatttgcaagccaagatcaaggctgcccaataccaggtcaaccaggctgcagctgcacaggctgcagccaccgcagctgccatgactcagtcggctgtcaaatcactgaagcgacccctcgaggcaacctttgacctgggaattcctcaagctgtacttcccccattaccaaagaggcctgctcttgaaaaaaccaacggtgccaccgcagtctttaacactggtattttccaataccaacaggctctagccaacatgcagttacaacagcatacagcatttctcccaccaggctcaatattgtgcatgacacccgctacaagtgttgttcccatggtgcacggtgctacgccagccactgtgtccgcagcaacaacatctgccacaagtgttcccttcgctgcaacagccacagccaaccagatacccataatatctgccgaacatctgactagccacaagtatgttacccagatgtag

SEQ ID NO: 56 - DNA, 1203 bp, Artificial Sequence
  Description of Artificial Sequence Synthetic polynucleotide
atggcagtgagcgtgaccccgataagagacacgaagtggctgactctggaggtctgccgggaatttcagagaggtacatgtagcagaccggacacagaatgcaagtttgctcatcctagcaaatcttgtcaggtcgagaacggaagggttattgcgtgcttcgactcactcaaaggacgctgcagtcgcgagaactgcaagtaccttcatccacctccgcacttgaaaacgcagcttgagatcaatgggaggaataaccttatacaacaaaagaatatggcaatgcttgcgcaacagatgcagctggctaatgccatgatgcctggggcaccgctgcaaccggttccaatgttctccgttgctccttctctggctacgaatgcttccgcagccgccttcaatccttatctgggccccgtttccccaagcctggtacccgcagaaatcctgccaactgctcctatgttggttaccggcaaccccggtgtgcccgttccggccgccgcagcagcagccgcccaaaaacttatgcggacagacaggctggaggtgtgtcgggaatatcagcgcggaaattgcaatcggggagaaaatgactgtagatttgcccacccagcagacagtacgatgatcgacacgaatgataacactgtaaccgtgtgcatggattatattaaaggtaggtgcagcagggagaagtgcaagtattttcatccacccgcccatctgcaagcaaagatcaaggcggcacaatatcaagtcaatcaagccgctgcggcgcaagccgcagcgacagcagccgctatgacacagtcagccgtaaagtctctgaaaaggcctttggaagccactttcgatttggggatacctcaagcagtacttccaccgctgcccaagcgaccggcgttggaaaagacaaacggtgctacagcagtgttcaataccggcatattccaatatcagcaagcactcgcaaatatgcagctccagcagcacacggctttccttccgccaggctcaatcctttgcatgactccagctacatcagttgtaccgatggtgcatggagccacaccggcgactgtgtctgccgcgactacctcagccacaagcgtcccctttgcggccaccgccactgccaaccagataccgattatttctgcagaacacttgacttcacacaagtatgtcacccaaatgtag

SEQ ID NO: 57 - DNA, 1899 bp, Homo sapiens
atggaggagctggtggtggaagtgcggggctccaatggcgctttctacaaggcatttgtaaaggatgttcatgaagattcaataacagttgcatttgaaaacaactggcagcctgataggcagattccatttcatgatgtcagattcccacctcctgtaggttataataaagatataaatgaaagtgatgaagttgaggtgtattccagagcaaatgaaaaagagccttgctgttggtggttagctaaagtgaggatgataaagggtgagttttatgtgatagaatatgcagcatgtgatgcaacttacaatgaaattgtcacaattgaacgtctaagatctgttaatcccaacaaacctgccacaaaagatactttccataagatcaagctggatgtgccagaagacttacggcaaatgtgtgccaaagaggcggcacataaggattttaaaaaggcagttggtgccttttctgtaacttatgatccagaaaattatcagcttgtcattttgtccatcaatgaagtcacctcaaagcgagcacatatgctgattgacatgcactttcggagtctgcgcactaagttgtctctgataatgagaaatgaagaagctagtaagcagctggagagttcaaggcagcttgcctcgagatttcatgaacagtttatcgtaagagaagatctgatgggtctagctattggtactcatggtgctaatattcagcaagctagaaaagtacctggggtcactgctattgatctagatgaagatacctgcacatttcatatttatggagaggatcaggatgcagtgaaaaaagctagaagctttctcgaatttgctgaagatgtaatacaagttccaaggaacttagtaggcaaagtaataggaaaaaatggaaagctgattcaggagattgtggacaagtcaggagttgtgagggtgaggattgaggctgaaaatgagaaaaatgttccacaagaagaggaaattatgccaccaaattcccttccttccaataattcaagggttggacctaatgccccagaagaaaaaaaacatttagatataaaggaaaacagcacccatttttctcaacctaacagtacaaaagtccagagggtgttagtggcttcatcagttgtagcaggggaatcccagaaacctgaactcaaggcttggcagggtatggtaccatttgtttttgtgggaacaaaggacagcatcgctaatgccactgttcttttggattatcacctgaactatttaaaggaagtagaccagttgcgtttggagagattacaaattgatgagcagttgcgacagattggagctagttctagaccaccaccaaatcgtacagataaggaaaaaagctatgtgactgatgatggtcaaggaatgggtcgaggtagtagaccttacagaaatagggggcacggcagacgcggtcctggatatacttcaggaactaattctgaagcatcaaatgcttctgaaacagaatctgaccacagagacgaactcagtgattggtcattagctccaacagaggaagagagggagagcttcctgcgcagaggagacggacggcggcgtggagggggaggaagaggacaaggaggaagaggacgtggaggaggcttcaaaggaaacgacgatcactcccgaacagataatcgtccacgtaatccaagagaggctaaaggaagaacaacagatggatcccttcagatcagagttgactgcaataatgaaaggagtgtccacactaaaacattacagaatacctccagtgaaggtagtcggctgcgcacgggtaaagatcgtaaccagaagaaagagaagccagacagcgtggatggtcagcaaccactcgtgaatggagtaccctaa

SEQ ID NO: 58 - DNA, 1899 bp, Artificial Sequence
  Description of Artificial Sequence Synthetic polynucleotide
atggaggagttggttgtggaggtacgaggttcaaacggtgctttttataaggcgttcgtaaaggacgtacacgaagactcaataacagttgcattcgagaataattggcaacctgatcgacagatcccatttcacgacgtgaggtttccacccccagtgggctataataaggatatcaacgagtctgatgaggtggaggtatattctcgggcgaatgagaaagagccgtgttgctggtggctcgctaaagttcggatgatcaagggtgagttttatgtcatagaatatgcagcttgtgatgctacctataacgaaatcgtcacaattgagaggctcagatcagttaatccgaacaaacccgctactaaagatacgtttcacaagatcaaactcgacgtccctgaggaccttaggcaaatgtgtgcgaaagaagcagctcataaagactttaagaaagcagttggagcattcagcgttacgtatgatcctgaaaattaccagttggtcatcctgtctatcaacgaggttacgagcaaaagggcgcatatgctgattgatatgcatttccggagcttgcgcacgaaattgtctttgatcatgaggaacgaggaagcgtcaaagcagctcgaaagtagccggcagcttgcctcaagatttcatgaacagtttatcgtgcgggaagaccttatgggcctggcgatcggcacgcacggggctaatattcagcaggcccggaaagtgcctggtgtcaccgctatcgacctggatgaagacacttgcacctttcacatttatggtgaggaccaggacgcggtgaaaaaagctaggagttttctcgagttcgccgaggacgttatacaagttcccaggaaccttgtaggcaaagtaataggtaagaatgggaaactcatacaagagatagtcgataaaagtggcgtagtcagagtaaggatcgaggcggaaaatgagaaaaatgtaccccaagaggaagaaatcatgccaccaaacagtttgccgtctaacaacagtcgggtcggccccaatgcgccggaggaaaaaaagcatcttgatattaaagaaaatagcacacacttctcacaacccaattcaaccaaagtgcaacgagttcttgtagcttcatcagtcgttgccggcgaatctcaaaaaccagaactcaaagcctggcaaggtatggtcccattcgtatttgtgggtacgaaggactctatcgccaacgccaccgtactcctcgactaccacctgaactacctgaaggaagttgatcagcttcgcctcgagcgcttgcaaatagacgagcagttgcggcagataggagcaagcagtaggcccccaccaaacaggacagataaagaaaaaagctacgttacggatgacggtcaaggaatgggccgcggcagccgaccatataggaatcgcgggcatggtcgcagaggacccggttacacctcaggcaccaacagcgaggcctcaaacgcctcagaaacggagagcgaccatagggatgaactttcagactggtccttggctccgactgaggaggaacgggagagtttcttgcggagaggtgatggccgcaggagagggggtggggggcgaggtcaaggaggacggggcagaggaggcggatttaagggcaacgatgatcactcaaggaccgataataggccaagaaatccgcgcgaagcgaaagggaggactacagacgggagcttgcaaataagagtcgattgcaacaatgagcggtccgtgcacacaaaaacccttcaaaatacctcatccgagggttcccgcctgcgaacaggcaaagacaggaatcaaaagaaagagaaaccagattcagtcgacggacaacaacctttggttaacggtgtgccctaa

SEQ ID NO: 59 - DNA, 66 bp, Artificial Sequence
  Description of Artificial Sequence Synthetic oligonucleotide
gatcgcaattttcagatttgcacaaatcaagagtttgtgcaaatctgaaaattgttttttgaagct

SEQ ID NO: 60 - DNA, 72 bp, Artificial Sequence
  Description of Artificial Sequence Synthetic oligonucleotide
gatcgcaattttcagatttgcacaaagccctgacccagctttgtgcaaatctgaaaattgttttttgaagct

SEQ ID NO: 61 - DNA, 140 bp, Artificial Sequence
  Description of Artificial Sequence Synthetic polynucleotide
aacagaaggctcgagaaggtatattgctgttgacagtgagcgcaattttcagatttgcacaaatagtgaagccacagatgtatttgtgcaaatctgaaaattgtgcctactgcctcggacttcaaggggctagaattcga